(19) United States
(12) Patent Application Publication
(10) Pub. No.: US 2026/0002941 A1
(43) Pub. Date: Jan. 1, 2026

(54) SINGLE-CELL PROTEOMICS ANALYSIS OF EMBRYOS

(71) Applicants: California Institute of Technology, Pasadena, CA (US); Northeastern University, Boston, MA (US)

(72) Inventors: Magdalena D. Zernicka-Goetz, Pasadena, CA (US); Lisa K. Iwamoto-Stohl, Pasadena, CA (US); Aleksandra A. Petelski, Boston, MA (US); Nikolai G. Slavov, Boston, MA (US)

(21) Appl. No.: 19/249,653

(22) Filed: Jun. 25, 2025

Related U.S. Application Data
(60) Provisional application No. 63/665,046, filed on Jun. 27, 2024.

(51) Int. Cl. G01N 33/68; C12N 5/073

(52) U.S. Cl. CPC G01N 33/6848; C12N 5/0603; G01N 2570/00

(57) ABSTRACT

Provided herein include methods and compositions for single-cell proteomics analysis of mammalian embryos and uses thereof to screen perturbations and to detect perturbation-induced change in the protein composition of the mammalian embryos.

### RELATED APPLICATIONS

This application claims the benefit under 35 U.S.C. § 119(e) of U.S. Provisional Patent Application Ser. No. 63/665,046, filed on Jun. 27, 2024, the content of this related application is incorporated herein by reference in its entirety for all purposes.

### STATEMENT REGARDING FEDERALLY SPONSORED R&D

This invention was made with government support under Grant No(s). GM123497 & GM144967 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810011-US_SeqList, created Jun. 1, 2025, which is 20,664 bytes in size. The information in the electronic format of the Sequence Listing is incorporated herein by reference in its entirety.

### BACKGROUND

### Field

The present disclosure relates generally to the field of embryo development and related testing.

### Description of the Related Art

Pre-patterning of the embryo, driven by spatially localized factors, is a common feature across several non-mammalian species. However, mammals display regulative development and thus it was thought that blastomeres of the embryo do not show such pre-patterning, contributing randomly to the three lineages of the blastocyst: the epiblast, primitive endoderm and trophectoderm that will generate the new organism, the yolk sac and placenta respectively. Unexpectedly, early blastomeres of mouse and human embryos have been reported to have distinct developmental fates, potential and heterogeneous abundance of certain transcripts. Nevertheless, the extent of the earliest intra-embryo differences remains unclear and controversial. There remains a need to understand intra-zygotic and inter-blastomere proteomic asymmetry in mammals, which can facilitate the assessment of the development potential of embryo cells and of the health status of an embryo in IVF clinics.

### SUMMARY

Disclosed herein include methods for determining protein composition of a mammalian embryo in vitro. In some embodiments, the method comprises: culturing one or more mammalian embryos at the zygote stage in an embryo culture media until the one or more mammalian embryos reach at least early 2-cell stage; separating sister blastomeres of a mammalian embryo into single blastomeres; subjecting the single blastomeres to single-cell mass-spectrometry to obtain a proteomic profile of each single blastomere; and identifying a first set of proteins differentially abundant in a first single blastomere and a second set of proteins differentially abundant in a second single blastomere, both blastomeres derived from a same mammalian embryo.

In some embodiments, the one or more mammalian embryos at the zygote stage are cultured in the embryo culture media for about 1-3 hours. In some embodiments, the one or more mammalian embryos reach a late 2-cell blastomere stage. In some embodiments, the one or more embryos is cultured in the embryo culture media for about 17-19 hours. In some embodiments, the one or more mammalian embryos reach a 4-cell blastomere stage. In some embodiments, the one or more mammalian embryos are cultured in the embryo culture media for about 26-28 hours. In some embodiments, the one or more mammalian embryos reach a blastocyst stage or an 8-cell blastomere stage. In some embodiments, the one or more mammalian embryos at the zygote stage are cultured in the embryo culture media for about 6-12 hours.

In some embodiments, the embryo culture media comprises physiological salts, a carbon source, an antibiotic, and a buffer. In some embodiments, the carbon source is glucose. In some embodiments, the antibiotic comprises Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. In some embodiments, the embryo culture media further comprises non-human serum or serum substitute. In some embodiments, the non-human serum or serum substitute comprises fetal bovine serum, bovine serum albumin, human serum albumin, or any combination thereof. In some embodiments, the embryo culture media comprises physiological salts, energy substrates, bicarbonate or HEPES, essential amino acids, glutamine dipeptide, human serum albumin, EDTA, gentamicin, or any combination thereof. In some embodiments, the embryo culture media comprises sodium chloride, potassium chloride, calcium chloride, potassium phosphate, magnesium sulfate, sodium bicarbonate, glucose, sodium lactate, sodium pyruvate, amino acids, EDTA, gentamicin sulfate, or any combination thereof.

In some embodiments, separating blastomeres of the one or more mammalian embryos into single blastomeres comprises removing the zona pellucida of the one or more mammalian embryos and bisecting the embryos. In some embodiments, the method comprises washing the single blastomeres. In some embodiments, subjecting the single blastomeres to single-cell mass-spectrometry comprises lysing the single blastomeres. In some embodiments, the method comprises using a physical or mechanical lysis process, and contacting the cell lysate with a trypsin enzyme. In some embodiments, subjecting the single blastomeres to single-cell mass-spectrometry comprises labeling digested peptides by isobaric mass tags (TMT). In some embodiments, the single-cell mass-spectrometry comprises preparing an isobaric carrier. In some embodiments, preparing the isobaric carrier comprises lysing embryonic stem cells and contacting the cell lysate with a trypsin enzyme. In some embodiments, the single-cell mass-spectrometry is label-free. In some embodiments, the single-cell mass-spectrometry comprising using SCoPE2 method. In some embodiments, the single-cell mass-spectrometry employs data-independent acquisition approach or data-dependent acquisition approach.

The method can comprise determining the degree of asymmetry in the single blastomeres derived from the same mammalian embryo. In some embodiments, the single blastomeres are derived from a mammalian embryo at a two-cell stage. In some embodiments, the single blastomeres are derived from a mammalian embryo at an early two-cell stage or a late two-cell stage, at a four-cell stage, or at an eight-cell stage. The method can comprise comparing the degrees of asymmetry of single blastomeres derived from mammalian embryos across different developmental stages. The method can comprise identifying biological processes that are differential among the single blastomeres derived from the same mammalian embryo. The method can comprise performing single-cell RNA-sequencing for early, mid, and late two-cell stage blastomeres.

In some embodiments, the second set of proteins differentially abundant in the second single blastomere comprise proteins that support the epiblast formation and suppress the trophectoderm formation. The method can comprise identifying the single blastomere having a higher developmental potential. In some embodiments, the first set of proteins differentially abundant in the first single blastomere comprises PSMC4, and the second set of proteins differentially abundant in the second single blastomere comprises Nedd8 and Gps1.

Disclosed herein include methods for determining protein composition of a mammalian embryo at the zygote stage in vitro. In some embodiments, the method comprises: splitting a zygote stage mammalian embryo into two zygote halves; subjecting the two zygote halves to single-cell mass-spectrometry to obtain a proteomic profile of each zygote half; and identifying a first set of proteins differentially abundant in a first zygote half and a second set of proteins differentially abundant in a second zygote half.

In some embodiments, the splitting is meridionally in alignment with the animal-vegetal axis as defined by the position of the polar body. In some embodiments, the splitting is equatorial, and perpendicular to animal-vegetal axis. The method can comprise washing the two zygote halves prior to the single-cell mass-spectrometry. In some embodiments, subjecting the two zygote halves to single-cell mass-spectrometry comprises lysing the two zygote halves, e.g., via a physical or mechanical lysis, and contacting the cell lysate with a trypsin enzyme. In some embodiments, subjecting the two zygote halves to single-cell mass-spectrometry comprises preparing an isobaric carrier. In some embodiments, the isobaric carrier comprises embryonic stem cells. In some embodiments, the embryo is a human embryo. In some embodiments, the embryo is a mouse embryo.

Disclosed herein include methods for detecting perturbation-induced change in mammalian embryo cells. In some embodiments, the method comprises: introducing a perturbation to a mammalian embryo at the zygote stage; determining a protein composition of the mammalian embryo according to any method of the disclosure; and detecting the perturbation-induced change in the protein composition of single blastomeres, optionally the determining comprises comparing the protein composition obtained in the presence of the perturbation with a protein composition obtained in the absence of the perturbation.

In some embodiments, the perturbation is a physical condition or a chemical condition. In some embodiments, the perturbation comprises a genetic perturbation, a drug candidate, an absence or presence of a test agent, an increase or decrease of temperature, light, pressure, pH value, or a combination thereof. In some embodiments, introducing the perturbation to the mammalian embryo comprises contacting the mammalian embryo with the perturbation in a culture media or culturing the mammalian embryo in a culture media in the presence of the perturbation. In some embodiments, introducing the perturbation to the mammalian embryo comprises performing RNA interference or CRISPR-Cas gene editing. In some embodiments, the single blastomeres are separated from a two-cell stage mammalian embryo or a four-cell stage mammalian embryo. In some embodiments, detecting the perturbation-induced change on the protein composition of sister blastomeres comprise detecting the abundance of one or more proteins selected from Nedd8, Gps1, PSMC4, or a combination thereof in the sister blastomeres.

Disclosed herein include methods of selecting blastomeres from mammalian embryos. In some embodiments, the method comprises: culturing one or more mammalian embryos at the zygote stage in an embryo culture media until the one or more mammalian embryo reaches 2-cell stage or 4-cell stage, separating sister blastomeres of a mammalian embryo into single blastomeres; detecting the abundance of one or more proteins selected from Nedd8, Gps1, PSMC4 or any combination thereof in the single blastomeres; and selecting one or more single blastomeres based on the abundance of the one or more proteins selected from Nedd8, Gps1, PSMC4, or any combination thereof.

In some embodiments, the selecting comprises comparing the abundance of the one or more proteins with a reference level. In some embodiments, the one or more proteins comprise beta proteins, alpha proteins, or both. In some embodiments, the mammalian embryos are human embryos. In some embodiments, the mammalian embryos are mouse embryos.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 displays non-limiting exemplary data related to proteomic asymmetry at the 2- and 4-cell stage mouse embryos. Panel a, A schematic of pre-implantation development. Following a series of cleavage divisions, the embryo polarizes at the 8-cell stage and undergoes a series of asymmetric and symmetric divisions to give rise to the inner cell mass (ICM) and outer trophectoderm (TE) cells. The ICM then gives rise to the epiblast (EPI) and primitive endoderm (PE). Samples were collected at the indicated timepoints. hCG, human chorionic gonadotrophin. Panel b, A schematic showing the experimental harvesting of single blastomeres from 2-cell (top) and 4-cell stage (bottom) embryos for single-cell proteomics analysis. ZP, zona pellucida. Panel c, Representative images of embryos prior to and following splitting into individual blastomeres. Scale bars, 40 μm. Panel d, K-means clustering of 2-cell stage blastomeres results in a consistent bi-clustering of sister blastomeres, i.e., sisters from the same embryo fall into opposing clusters, which are termed alpha and beta. Heatmaps of ˜300 proteins with differential abundance in alpha and beta cells. Panel e, By changing the starting centroids in the k-means clustering approach 200 times, vectors of cell cluster classification were obtained for each iteration. This allows us to determine the probability of cells landing in the same cluster (alpha or beta). The majority of embryos consistently fall into the same cluster, indicating that the clustering approach is stable.

FIG. 2 displays non-limiting exemplary data showing that proteomic asymmetry is inherited from the zygote. Panel a, Schematic illustrating the collection of zygotes and subsequent meridional cutting according to the animal-vegetal axis as defined by the position of the second polar body (PB). ZP, zona pellucida. Panel b, Representative images of zygotes prior to and following splitting into individual halves. Scale bars, 40 μm. Panel c, Principal Component Analysis (PCA) of the zygote halves shows a biclustering pattern. Each zygote pair lands in separate clusters. Panel d, The bars on the top show the difference between the PC1 loadings corresponding to each zygote pair ordered in a descending order. On bottom, pairwise spearman correlations were computed between each zygote pair and the 2-cell stage embryos. The correlations were computed on vectors of fold changes of proteins that were both significantly differential between alpha and beta cells and quantified in the zygote dataset. Median correlations of each distribution are shown by triangles.

FIG. 3 displays non-limiting exemplary data showing that alpha and beta blastomere clusters exhibit differential biological processes. Panels a, b, PSEA analysis revealed differential abundance of proteins related to specific biological processes between alpha and beta cell clusters, namely protein degradation and protein transport. Panel c, Representative scatter plot of raw reporter ion intensities from one representative blastomere versus 200 ESCs on the log10 scale. Points correspond to peptides of proteins mapping to the subcortical maternal complex or peptides of proteins mapping to different ubiquitin ligases as indicated. The diagonal line represents a separation between the two clusters. Such scatterplots were observed for blastomeres across the stages. Upon systematic analysis of all blastomeres in all stages, proteins involved in protein degradation and transport were found to be heavily enriched in blastomeres as compared to mouse ESCs.

FIG. 4 displays non-limiting exemplary data related to the manipulation of two beta proteins impacts lineage composition. Panel a, Schematic of clonal dsRNA-mediated knockdown (KD) or mRNA-mediated overexpression (OE) of candidates. One blastomere of 2-cell stage embryos was injected with dsRNA targeting candidates or eGFP (control) and mRNA for the membrane marker Gap43-RFP for KD experiments. For OE experiments one blastomere of 2-cell stage embryos was injected with mRNA for overexpression of candidates (at the indicated concentration) and for the membrane marker Gap43-RFP. Embryos were cultured to the late blastocyst stage and the contribution of the Gap43-RFP-positive cells to each cell lineage analyzed for both OE and KD experiments. Panel b, Representative images of control (ds-eGFP), dsNedd8 and dsGps1 blastocysts. Scale bar, 20 μm. Panel c, Representative images of control (Gap43-RFP), Nedd8-HA overexpression (OE) and Gps1-HA OE blastocysts. Scale bar, 20 μm. Panel d, dsNedd8 cells show increased contribution to the trophectoderm (TE) lineage. Contribution of dsNedd8 cells to the trophectoderm (TE, Cdx2 positive), primitive endoderm (PE, Sox17 positive), and epiblast (EPI, double negative), was assessed relative to control embryos. Control n=27 embryos, dsNedd8 n=36 embryos. Mann-Whitney test, ***p=0.0005. Panel e, Nedd8-HA OE cells show decreased contribution to the TE lineage. Contribution of Nedd8-HA OE cells to the TE, PE and EPI was assessed relative to control embryos. Control n=36 embryos, Nedd8-HA OE 50 ng/μl n=11 embryos, Nedd8-HA OE 500 ng/μl n=28 embryos. Ordinary one-way ANOVA test, adjusted p values, *p=0.0375 (TE), 0.0120 (PE) and 0.0445 (EPI). Panel f, dsGps1 cells show significantly reduced contribution to the EPI. Contribution of dsGps1 cells to the TE, PE and EPI was assessed relative to control embryos. Control n=33 embryos, dsGps1 n=35 embryos. Mann-Whitney test, *p=0.0274, ****p<0.0001. Panel g, Gps1-HA OE cells show significantly increased contribution to the EPI. Contribution of Gps1-HA OE cells to the TE, PE and EPI was assessed relative to control embryos. Control n=17 embryos, Gps1-HA OE 50 ng/μl n=12 embryos, Gps1-HA OE 500 ng/μl n=20 embryos. Kruskal-Wallis test, adjusted p values, *p=0.0297. For d-g, data are shown as mean±s.e.m.

FIG. 5 displays non-limiting exemplary data showing that beta cells have a higher developmental potential. Panel a, Schematic illustrating the collection of one sister blastomere for single cell proteomics analysis and subsequent culturing of the other sister to the blastocyst stage. Panel b, Representative images of blastocysts with 4 or more epiblast (EPI) cells and fewer than 4 EPI cells. Images are shown as maximum projections and representative single plane zooms showing the composition of the inner cell mass. Panel c, Normalized EPI cell counts trend positively with sister cells' alpha-beta polarization. Plot shows paired blastomere data that was filtered for the sister blastocyst's characteristics, i.e., at least 10 cells total and have only zero or one lineage totally absent. The size of the data points corresponds to the total number of cells present in the resultant embryo from the blastomere that was left in culture. The color of the data points corresponds to the presence of the three lineages: purple dots mark a blastocyst that contained all three lineages, while brown dots mark a resultant blastocyst that contained at least two lineages. The relationship between the number of epiblast cells in the resulting blastocyst and the corresponding sister's alpha-beta polarization is quantified by a Pearson correlation computed using all displayed datapoints. Panel d, Violin plots of healthy vs less healthy blastocysts and their sister's alpha-beta polarization. Only blastocysts with more than 10 cells and at most 1 lineage absent were included in this analysis. Healthy blastocysts are defined as having at least 4 epiblast cells, less healthy as having 3 or fewer epiblast cells. The size of the points is proportional to the total cell count of the resulting blastocyst that was imaged. The statistical significance of this result was tested with a t-test that had a resulting p-value of 0.005. Panel e, Heatmap showing the pairwise cell correlations (based on vectors of alpha-beta proteins that exhibited high fold-change), for 4-cell stage blastomeres. Each tile represents a correlation value between two blastomeres, while the color bars below indicate whether the blastomere was identified as a vegetal cell, and its alpha-beta polarization. From this analysis, two clusters of blastomeres can be observed corresponding to alpha and beta. Vegetal cells are significantly more likely to cluster with the alpha-like cells (p=0.047, as calculated using the hypergeometric distribution probability).

FIG. 6 displays non-limiting exemplary data showing that alpha and beta clusters are conserved in human embryos at the 2-cell stage. Panel a, Representative images of human 2-cell embryos prior to and following splitting into individual blastomeres. Scale bars, 40 μm. Panel b, Dendrogram illustrating the biclustering behavior in all processed human 2-cell stage blastomeres. Embryo numbers are for indexing purposes only. Color coding indicates the cluster in which each blastomere is classified. Panel c, Heatmap of the 113 proteins that are differentially abundant between the two cell clusters. Human blastomeres on the x-axis are ordered in the same way as the dendrogram, while the proteins on the y-axis have been ordered through hierarchical clustering. Panel d, Extracted ion chromatogram of peptide mapping to VDAC2 on both the MS1 and MS2 levels indicate consistent fold change between sister cells. Panel e, Boxplots of fold changes between sisters of proteins contributing to protein degradation and protein transport terms, which were found to be significantly differential between alpha and beta cells in the human 2-cell stage dataset. Panel f, Heatmap of pairwise correlations among mouse and human 2-cell stage embryos based on all intersected proteins shows two clusters, and hence, the level of agreement between alpha and beta classification is positive. Panel g, Heatmap of intersected GO terms that are significantly differential between mouse and human (each value represents z-score of median protein abundance for each GO term in each blastomere).

FIG. 7 displays non-limiting exemplary data related to proteomic asymmetry in mouse blastomeres. Panel a, The number of clusters (k) that can best explain the data plotted against the average silhouette width. In this case, k=2 provides the best explanation for the data. Panel b, Representative density plot showing quantitation variability for peptides mapping to the same protein in each mouse blastomere. Panel c, Spearman correlation plot of all individual blastomeres, from the 2-cell and 4-cell stages, which demonstrates the presence of two clusters. Panel d, The level of variance of alpha-beta protein quantitation in each blastomere at the 4-cell stage. Each grouping of 4 blastomeres represents an embryo on the x-axis. The y-axis is the variance of alpha and beta protein abundances in each blastomere. All blastomeres that were part of the same embryo are colored in the same color. Panel e, Variability of alpha-protein quantitation among sisters in each 4-cell stage embryo Each grouping of 4 blastomeres represents an embryo on the x-axis. The y-axis is the fold change between the mean abundances of alpha proteins and beta proteins in each blastomere. All blastomeres that were part of the same embryo are colored in the same color.

FIG. 8 displays non-limiting exemplary data related to cleavage pattern analysis between Alpha and Beta cells. Panel a, Schematic of division patterns from the 2- to 4-cell stage and representative stills from live imaging to classify division pattern. E denotes equatorial division and M meridional in relation to the animal-vegetal axis of the fertilized egg, with the first letter denoting the first cleavage and the second letter the second. ME (M-division followed by E-division); EM (E-division followed by M-division); MM (consecutive M-divisions); EE (consecutive E-divisions). The position of the polar body is indicated with an asterisk. Scale bar, 20 μm. Panel b, Schematic showing the experimental harvesting of single blastomeres from 4-cell stage embryos, with classified division pattern and order, which were subsequently prepared using SCoPE2. Division pattern and order were classified by live imaging, following microinjection of synthetic mCherry mRNA to label one of the two sisters at the 2-cell stage. ZP, zona pellucida. Panel c, Q-value distribution of protein-level analysis and GO term-level analysis. Dotted line indicated q.value=0.05 Panel d, Proteins within the 5% FDR threshold in determining differences between alpha and beta cell clusters within the scope of cleavage patterns. Panel e, GO terms that passed the 5% FDR threshold in determining differences between alpha and beta cell clusters within the scope of cleavage patterns.

FIG. 9 displays non-limiting exemplary data showing that post-transcriptional mechanisms and maternal contributions may underlie proteomic asymmetries. Panel a, Scatterplot of median protein fold changes between zygote halves on the x-axis and median protein fold changes between sister blastomeres at the two-cell stage. The proteins chosen were both differentially abundant between alpha- and beta-type cells at the two-cell stage and quantified in the zygote dataset. A positive correlation of −0.45 was observed, which is highly significant (p-value<1e-8). Panel b, Uniform manifold approximation and projection (UMAP) of single cell transcriptome of 2-cell embryos from the Deng et al. dataset. Individual cells are coloured based upon stage: Early 2-cell in red, mid 2-cell in light green, and late 2-cell in light blue. Panels c, d, UMAP portraying either an Alpha (c) or Beta (d) score for each cell. Panels e, f, Clustermap displaying expression levels for Alpha (e) or Beta (f) transcripts in each blastomere. Sister blastomeres appear next to one another along the vertical axis; blastomeres are labelled by stage, embryo number, and blastomere number, respectively. Panel g, Heat map showing Alpha or Beta scores for each blastomere. Blastomeres from the same embryo are grouped together. Panel h, Heatmap showing median transcript abundance mapping to particular GO terms that are known to have heterogeneity between blastomeres in the 2-cell embryo based on proteome data. Blastomeres of the same embryo are plotted next to each other.

FIG. 10 displays non-limiting exemplary data showing temporal overview of differences between alpha and beta cells. Panel a, P values of the top most enriched processes in blastomeres relative to ESCs. “Protein catabolic”, “catabolic”, “ubiquitin-dependent protein catabolic”, “modification-dependent protein catabolic”, and “proteolysis involved in cellular protein catabolic” corresponds to protein degradation processes, while “ER to Golgi vesicle-mediated transport” and “golgi vesicle transport” corresponds to protein transport processes. Panel b, Boxplots illustrating the levels of significantly differential (1% FDR) Ribosomal Proteins (RPs) between alpha and beta cells. The color of the boxplots corresponds to the developmental stage. RPs were tested separately between alpha and beta cells, and for each stage. Panel c, Density plots of protein translation themes that were found significantly differential between alpha and beta cell clusters. Panel d, Euclidean distance of normalized protein abundance between each blastomere in each embryo, showing increasing inter-blastomere differences. Panels e, f, Correlation values of top protein sets (by highest absolute correlation value) obtained from analysis across the stages.

FIG. 11 displays non-limiting exemplary data showing differential alpha and beta blastomere clusters. Panel a, Representative images of further examples of control (ds-eGFP), dsNedd8, dsGps1 and of dsPSMC4 blastocysts. Scale bar, 20 μm. Panel b, Bar chart showing the average total number of cells and the proportion of RFP positive or negative cells in control and dsNedd8 late blastocysts. Mann-Whitney test, ****p<0.0001. Panel c, dsNedd8 cells show increased contribution to the blastocyst stage embryo. Control n=27 embryos, dsNedd8 n=36 embryos. Mann-Whitney test, *p=0.0024. Panel d, Bar chart showing the average total number of cells and the proportion of RFP positive or negative cells in control and dsGps1 late blastocysts. Panel e, dsGps1 cells show significantly reduced contribution to blastocyst stage embryo. Control n=33 embryos, dsGps1 n=35 embryos. Mann-Whitney test, **p=0.0081. Panel f, Bar chart showing the average total number of cells and the proportion of RFP positive or negative cells in control and dsPSMC4 late blastocysts. Panel g, dsPSMC4 cells show decreased contribution to the blastocyst stage embryo. Mann-Whitney test, **p<0.001. Panel h, dsPSMC4 cells show decreased contribution to all three lineages. Contribution of dsPSMC4 cells to the trophectoderm (TE, Cdx2 positive), primitive endoderm (PE, Sox17 positive), and epiblast (EPI, double negative), was assessed relative to control embryos. Control n=10 embryos, dsPSMC4 n=16 embryos. Mann-Whitney test, *p=0.0449 (PE), *p=0.0475 (EPI), **p<0.0017. Data are shown as mean±s.e.m. Panel i, Bar chart showing the average total number of cells and the proportion of RFP positive or negative cells in control and Nedd8-HA OE late blastocysts. Panel j, Nedd8-HA OE cells show no difference in contribution to blastocyst stage embryo. Control n=36 embryos, Nedd8-HA OE 50 ng/μl n=11 embryos and Nedd8-HA OE 500 ng/μl n=28embryos. k, Bar chart showing the average total number of cells and the proportion of RFP positive or negative cells in control and Gps1-HA OE late blastocysts. 1, Gps1-HA OE cells show no difference in contribution to blastocyst stage embryo. Control n=17 embryos, Gps1-HA OE 50 ng/μl n=12 embryos, Gps1-HA OE 500 ng/μl n=20 embryos. The proportion of Gap43-RFP positive cells was assessed in control and mRNA injected embryos in c, e, g, j and l. For c, e, g, j and l, data are shown as individual data points on a Box and Whiskers plot.

FIG. 12 displays non-limiting exemplary data relate to the validation of knockdown and overexpression experiments. Panel a, Schematic for validation of dsRNA mediated knockdown. Embryos were injected with dsRNA targeting candidates (Nedd8=dsNedd8, Gps1=dsGps1, PSMC4=dsPSMC4) or eGFP (control) and collected after 48 hrs for qRT-PCR. Panel b, Nedd8 mRNA expression was assessed relative to control embryos. Control n=14 embryos, dsNedd8 n=14 embryos. Panel c, Gps1 mRNA expression was assessed relative to control embryos. Control n=15 embryos, dsGps1 n=15 embryos. Panel d, PSMC4 mRNA expression was assessed relative to control embryos. Control n=14 embryos, dsPSMC4 n=14 embryos. Panel e, Schematic for validation of mRNA mediated overexpression. Embryos were injected with mRNA for candidates, tagged with HA (Nedd8=Nedd8-HA OE, Gps1=Gps1-HA OE) or Gap43-GFP (control) and cultured for 24 hrs. Embryos were stained for HA and the normalized mean fluorescence intensity values of the Gap43-RFP positive cells and negative cells assessed. Panel f, Representative images of control (Gap43-RFP), Nedd8-HA overexpression (OE) and Gps1-HA OE 8-cell embryos. Scale bar, 20 μm. Panels g, h, i, HA expression of Gap43-GFP positive and negative cells was assessed in control, Nedd8-HA OE and Gps1-HA OE embryos respectively. Control n=11 embryos, RFP−n=40 cells, RFP+n=37 cells. Nedd8-HA OE n=12 embryos RFP−n=48 cells, RFP+n=42 cells. Gps1-HA OE n=11 embryos, RFP−n=42 cells, RFP+n=31 cells. Student's t test, *p=0.0109, ****p<0.0001. For g, h and i, data are shown as violin plots.

FIG. 13 displays non-limiting exemplary data showing difference in the development of sister 2-cell blastomeres. Panel a, Schematic of split embryo culture. 2-cell embryos were recovered and split before being cultured to the late blastocyst stage in pairs. Blastocysts were assessed for lineage marker expression. Panel b, Representative images of a pair of ‘twin’ blastocysts, showing which has more (high) or fewer (low) epiblast (EPI) cells. Images are shown as maximum projections showing the composition of the inner cell mass. Panel c, Embryos were classified as high or low EPI within each pair and the number of cells in each lineage (trophectoderm (TE, Cdx2 positive) epiblast (EPI, Nanog positive) and primitive endoderm (PE, SOX17 positive)). n=32 pairs of blastocysts. Panel d, Density plot showing the alpha-beta protein fold changes computed from global normalization. The blastomere type (‘alpha’ or ‘beta’) was defined by K-mean clustering after within embryo normalization for the 2-cell stage samples in which the proteomes of both sisters were analyzed. The alpha-beta fold change (x axis) was then computed from blastomere proteomes normalized to the mean across all single 2-cell blastomeres rather within each 2-cell embryo. Colour indicates the clustering based on normalization within each embryo, while the alpha-beta protein fold-change derived from normalizing across all blastomeres is shown on the x-axis. Panels e, f, Cell number in each lineage and proportion of each lineage respectively in blastocysts from FIG. 4 panels e-h. n=81embryos. Panel g, Scatterplot of number of total cells in resultant blastocyst versus alpha-beta polarization of sister cell analyzed by MS. Overall, there is no correlation between the two variables. Size of circles indicate the total number of cells in the imaged blastocyst, while the colors indicate the number of lineages present in the imaged blastocysts Panel h, Scatterplot of normalized primitive endoderm cell count in resultant blastocyst versus alpha-beta character of sister cell analyzed by MS. Overall, a negative correlation that is not statistically significant was observed. Size of circles indicate the total number of cells in the imaged blastocyst, while the colors indicate the number of lineages present in the imaged blastocysts. Panel i, Heatmap showing the pairwise cell correlations for all early 2-cell stage blastomeres. Correlations were calculated based on quantified alpha-beta proteins. Each tile represents a correlation value between two blastomeres, while the color bars below indicate alpha-beta polarization and whether the cell had an associated polar body. Cells with an identified polar body are more likely to cluster with beta cells when including all early 2-cell blastomeres (p=0.0036, as calculated using the hypergeometric distribution probability).

FIG. 14 displays non-limiting exemplary data showing that alpha-beta polarization does not relate to cell cycle asynchrony as assessed by PCNA expression. Panel a, Schematic of cell cycle analysis by live imaging of embryos expressing PCNA-clover. Zygotes were injected with PCNA-clover mRNA and allowed to cleave to the 2-cell stage. 2-cell embryos were split and imaged from 36 to 46 hrs post-hCG, during the transition from S to G2 phase, before being collected for single cell proteomics. Panel b, Representative still images of live imaging for PCNA-clover expression during the S/G2 transition. Red arrows indicate foci of PCNA-clover, which disappear as the cell enters G2. Magnified views are shown below. Time interval=15 mins. Panel c, Boxplots of alpha-beta polarization (derived from normalization across all single blastomeres) as distributed between blastomeres that exited the S phase either early or late. n=15 embryos, 30 cells. Panel d, Schematic of cell cycle analysis by live imaging of embryos expressing PCNA-clover, followed by culture to the blastocyst stage. Zygotes were injected with PCNA-clover and imaged as in a. Following a second cleavage division to the 4-cell stage, split embryos were cultured to the blastocyst stage and lineage composition assessed by immunofluorescence. Panel e, Epiblast and total cell number in blastocysts does not show any relation to S/G2 transition. Live imaging was used to determine which sister had ended S phase/entered G2 first or second. n=38 embryos/19 pairs.

FIG. 15 displays non-limiting exemplary data comparing human 2-cell data to mouse and stem cell derivative. Panels a, b Heatmaps of median fold-change between alpha and beta blastomeres of proteins that were found to be significantly differential between alpha and beta clusters in the mouse data. The first heatmap corresponds to proteins that are changing in the same direction across the human and mouse blastomeres. The second heatmap illustrates proteins that have opposing median levels between human and mouse blastomeres.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

For decades, it was thought that the blastomeres of mouse and human embryos are equivalent to each other in their developmental properties until reaching differential positions within the embryo at the 16-cell stage. However, the advent of new technologies to track individual cells in living embryos to determine their developmental fate and potential, and to examine the patterns of gene expression in single cells, has shown that blastomeres can become different from each other at earlier stages of development. Moreover, only one sister blastomere appears to be truly totipotent in the majority of 2-cell mouse embryos when sister blastomeres are separated from each other. A central question revolves around the molecular factors generating this heterogeneity. The present disclosure investigates intra-embryo differences at different developmental stages (e.g., from the zygote to the 4-cell stage), using single-cell MS proteomics for the first time. The single-cell proteomics approaches reveal the earliest incidence of proteomic asymmetry in mammalian embryos, which is correlated with developmental potential, providing novel insight into the role of early heterogeneity and cell fate.

Disclosed herein include in vitro methods and compositions for single-cell proteomics analysis of embryos at various developmental stages and uses thereof to detect perturbation-induced change in the protein composition of embryo cells. In some embodiments, a method for determining protein composition of an embryo in vitro is disclosed. The method can comprise culturing one or more embryos at the zygote stage in an embryo culture media until the one or more embryos reach at least early 2-cell blastomere stage, separating sister blastomeres of an embryo into single blastomeres, subjecting the single blastomeres to single-cell mass-spectrometry analysis to obtain a proteomic profile of each single blastomere, and identifying a first set of proteins differentially abundant in a first single blastomere and a second set of proteins differentially abundant in a second single blastomere, both blastomeres derived from a same embryo. The first single blastomere can be termed as alpha blastomere and the second single blastomere as beta blastomere.

Disclosed herein also includes a method for determining protein composition of an embryo at the zygote stage in vitro. The method can comprise splitting a zygote stage embryo into two zygote halves, subjecting the two zygote halves to single-cell mass-spectrometry analysis to obtain a proteomic profile of each zygote half, and identifying a first set of proteins differentially abundant in a first zygote half and a second set of proteins differentially abundant in a second zygote half.

Disclosed herein also includes a method for detecting perturbation-induced change in embryo cells. The method can comprise introducing a perturbation to an embryo at the zygote stage, determining a protein composition of the embryo according to the method described herein, and detecting the perturbation-induced change on the protein composition of single blastomeres, optionally the determining comprises comparing the protein composition obtained in the presence of the perturbation with a protein composition obtained in the absence of the perturbation.

Disclosed herein also includes a method of selecting blastomeres having a higher developmental potential. In some embodiments, a method of selecting blastomeres from mammalian embryos comprises culturing one or more embryos at the zygote stage in an embryo culture media until the one or more embryo reaches 2-cell stage or 4-cell stage, separating sister blastomeres of an embryo into single blastomeres, detecting the abundance of one or more proteins selected from Nedd8, Gps1, PSMC4, or a combination thereof in the single blastomeres, and selecting one or more single blastomeres based on the abundance level of the one or more proteins selected from Nedd8, Gps1, PSMC4, or a combination thereof.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100.

As used herein, the term “differentiation” can refer to the process by which an unspecialized (“uncommitted”) or less specialized cell acquires the features of a specialized cell such as, for example, a neuronal cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. As used herein, a “lineage-specific marker” can refer to a characteristic specifically associated with the phenotype of cells of a lineage of interest and can be used to assess the differentiation of an uncommitted cell to the lineage of interest.

The term “stem cell,” as used herein, refers to a cell that is capable of differentiating into one or more differentiated cell types. Stem cells may be totipotent. Stem cells may be pluripotent cells. Totipotent stem cells typically have the capacity to develop into any cell type. Totipotent stem cells are usually embryonic in origin. The term “progenitor cell,” as used herein, refers to a cell that is committed to a particular cell lineage and which gives rise to a particular limited range of differentiated cell types by a series of cell divisions. An example of a progenitor cell would be a myoblast, which is capable of differentiation to only one type of cell, but is itself not fully mature or fully differentiated.

The term “embryonic stem cell” (ES cell or ESC) as used herein refers to a pluripotent stem cell derived from the inner cell mass of a blastocyst, which is an early-stage preimplantation embryo. It is envisaged that such cells may express genes involved in the naive pluripotency network (Oct4/Nanog, Sox2, Klf4 etc.). Such cells may also have Oct4 proximal enhancer activity. They may contribute to all embryonic tissues in chimeras. The ES cells may be derived from mammalian embryos, obtained from iPS cells or obtained from appropriate cell lines. Non-limiting examples of said stem cells include embryonic stem cells of a mammal or the like established by culturing a pre-implantation early embryo, embryonic stem cells established by culturing an early embryo prepared by nuclear-transplanting the nucleus of a somatic cell, induced pluripotent stem cells (iPS cells) established by transferring several different transcriptional factors to a somatic cell, and pluripotent stem cells prepared by modifying a gene on a chromosome of embryonic stem cells or iPS cells using a gene engineering technique. More specifically, embryonic stem cells include embryonic stem cells established from an inner cell mass that constitutes an early embryo, embryonic stem cells established from a primordial germ cell, cells isolated from a cell population possessing the pluripotency of pre-implantation early embryos (for example, primordial ectoderm), and cells obtained by culturing these cells.

The term “trophoblast stem cell” as used herein refers to stem cells derived from the trophoblast lineage of an embryo. The trophoblast stem cells are preferably not extra-embryonic cells derived from the two cell types which are precursors of the human placenta: the cytotrophoblast and the syncitiotrophoblast. These cells can be derived at late pre-implantation stages E4.5 or early post-implantation stages (E5.5) but the resulting cell lines are equivalent to the stem cell compartment existing in the extra-embryonic ectoderm of the post-implantation mouse egg cylinder. Transcription factors such as Elf5, Eomes, and Tfap2C mark this lineage. TS cells can also be considered as cells that are the precursors of the differentiated cells of the placenta. In the mouse, TS cells can be derived from outgrowths of either blastocyst polar trophectoderm or extraembryonic ectoderm, which originates from polar trophectoderm after implantation.

As used herein, “markers”, “lineage markers” or, “lineage-specific markers” can refer to nucleic acid or polypeptide molecules that are differentially expressed in a cell of interest. Differential expression can mean an increased level for a positive marker and a decreased level for a negative marker as compared to an undifferentiated cell. The detectable level of the marker nucleic acid or polypeptide is sufficiently higher or lower in the cells of interest compared to other cells, such that the cell of interest can be identified and distinguished from other cells using any of a variety of methods known in the art. In some embodiments, a marker can be enriched or abundant. The term “enriched” or “abundant”, as used herein, shall have its ordinary meaning, and can also refer to a statistically significant increase in levels of a gene product (e.g., mRNA and/or protein) in one condition as compared to another condition (e.g., in one cell layer as compared to another cell layer).

The term, “concentration” as used herein shall have its ordinary meaning, and can also refer to (a) mass concentration, molar concentration, volume concentration, mass fraction, molar fraction or volume fraction, or (b) a ratio of the mass or volume of one component in a mixture or solution to the mass or volume of another component in the mixture or solution (e.g., ng/ml). In some embodiments, the concentration can refer to fraction of activity units per volume (e.g., U/ml).

The term “analogue” as used herein refers to a compound which may be structurally related to the relevant molecule. The term “agonist” as used herein can refer to a compound which might not be structurally related to the relevant molecule. For example, an agonist may activate the relevant receptor by altering the conformation of the receptor. Nevertheless, in both cases the terms are used in this specification to refer to compounds or molecules which can mimic, reproduce or otherwise generally substitute for the specific biological activity of the relevant molecule.

As used herein the phrase “culture medium” and “media” refers to a liquid substance used to support the growth and development of stem cells and of an embryo. The culture medium used according to some embodiments of the invention can be a water-based medium which includes a combination of substances such as salts, nutrients, minerals, vitamins, amino acids, nucleic acids, and/or proteins such as cytokines, growth factors and hormones needed for cell growth and embryo development.

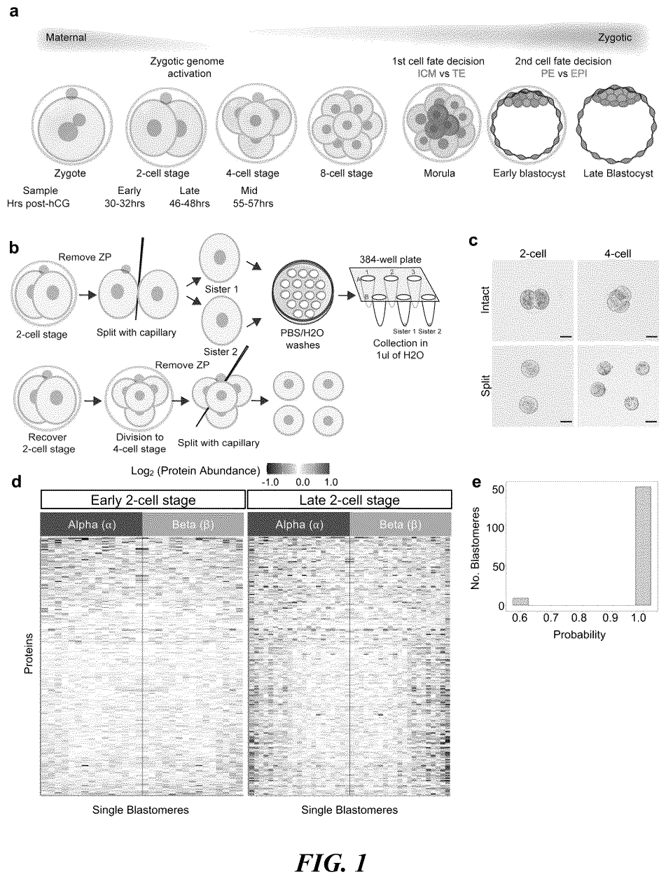

In mammals, including mouse and human, the fertilized egg undergoes cleavage divisions to give rise to embryonic and extraembryonic cell types. Two cell fate decisions are crucial for blastocyst formation. The first decision serves to generate outer cells, which will differentiate into the trophectoderm; and the inner cell mass (ICM), which will then undertake the second cell fate decision to give rise to epiblast and primitive endoderm (FIG. 1, panel a).

It has been long thought that all blastomeres had the same developmental potential to form these three lineages until the 16-cell stage. However, this view has changed over time, with multiple lines of evidence suggesting that totipotency is not only lost gradually, but also unevenly, in blastomeres before lineage specification. First, lineage tracing indicates that blastomeres of 2-cell mouse contribute unevenly to the ICM versus the trophectoderm in the blastocyst. Second, splitting of 2-cell mouse embryos into monozygotic twin “half embryos” found that one blastomere retains totipotency and gives rise to a live mouse, while the other cell fails to do so in the majority of cases. Lineage analyses of somatic mutations across human tissues with different developmental origins, including the placenta, suggest that such uneven contribution arising from the 2-cell blastomeres may also exist in humans. Indeed, the most recent lineage tracing studies have shown that, in most human embryos, the majority of the epiblast is derived from only one blastomere of the 2-cell embryo whereas the placenta is derived from both 2-cell stage blastomeres. However, the molecular basis for this asymmetry remains a long-standing question.

In other models, such as Drosophila melanogaster, symmetry-breaking mechanisms in the embryo involve asymmetric distribution of mRNAs and, in turn, the encoded proteins. Single-cell RNA sequencing methods have identified transcripts such as the non-coding RNA LincGET and Sox21 mRNA, to be differentially abundant in blastomeres of 2-cell and 4-cell stage mouse embryos respectively. However, asymmetries in the abundance of specific mRNAs between sister blastomeres may not be consistent and RNA abundance does not necessarily reflect protein abundance, as seen across tissues and during development.

To what extent the proteome differs between individual mammalian blastomeres remains unknown. Single-cell mass-spectrometry (MS) previously revealed proteomic differences between blastomeres of the Xenopus laevis embryo and among human oocytes. Bulk samples have been utilized to assess changes in the proteome during mouse embryo development but such bulk samples could not be used to discern intra-embryo heterogeneity.

The present disclosure investigates proteomic differences between single blastomeres from mammalian embryos (e.g., mouse and human) and their functional role. The present disclosure unexpectedly discovers early symmetry breaking of the protcome in the mammalian 2-cell embryo and even within the zygote. These proteome asymmetries can be used to predict the developmental potential of blastomeres.

In some embodiments, the present disclosure investigates intra-embryo differences during different developmental stages (e.g., from zygote to the 4-cell stage) using multiplexed and single-cell proteomics by mass-spectrometry (e.g., tandem mass tag labeled or label-free) and demonstrates that sister blastomeres from mouse and human embryos (e.g., 2-cell or 4-cell embryos) can be classified into two clusters, termed alpha and beta, defined by differential abundance of hundreds of proteins exhibiting strong functional enrichment for protein synthesis, transport, and degradation. Such asymmetrically distributed proteins include Gps1 and Nedd8, depletion or overexpression of which in one blastomere of the 2-cell embryo impacts lineage segregation. The data reveal that proteins involved in protein degradation and protein transport are highly enriched in blastomeres and differentially abundant across alpha and beta cells. These processes can be involved in symmetry breaking in the embryo, and in divergence in developmental potential prior to lineage diversification. These protein asymmetries increase at 4-cell stage relative to 2-cell stage. Halved mouse zygotes also display asymmetric protein abundance that resembles alpha and beta blastomeres, suggesting differential proteome localization already present within zygotes before zygotic genome activation.

The present disclosure also demonstrates that beta blastomeres have a higher developmental potential and can give rise to a blastocyst with more epiblast cells than alpha blastomeres. Furthermore, the present study also observed that vegetal 4-cell stage blastomeres, which are known to have a lower developmental potential are more likely to be alpha, linking the proteomic asymmetry found between sister cells in the earliest stages of development to eventual developmental fate. Finally, proteomic asymmetry appears to be conserved in human 2-cell embryos and that the alpha and beta classification can be applied to human blastomeres. The present disclosure is the first demonstration of intra-zygotic and inter-blastomere proteomic asymmetry in mammals that has a role in lineage segregation.

It is worth noting that the collection of such samples is not trivial. All matched blastomeres from each embryo and individual zygote halves must first be separated from each other and then thoroughly washed in pure water without lysing to obtain “clean” MS data, without contaminating spectra from embryo culture medium and allowing for downstream intra-embryo analyses. The use of isobaric mass tags makes it challenging to estimate the reliability of quantification of each protein in each single cell, especially for proteins represented by a single peptide. This challenge can be mitigated in the samples analyzed by DIA (Data-independent Acquisition) and can be further mitigated in future studies using plexDIA. Furthermore, utilizing both DDA (Data-Dependent Acquisition) and DIA, two orthogonal methods, a pattern of asymmetry in the human 2-cell and mouse 4-cell embryos are established, that recapitulates the alpha-beta asymmetry observed in mouse.

Some of the methods and compositions disclosed herein are also disclosed in Iwamoto-Stohl L K et al. “Proteome asymmetry in mouse and human embryos before fate specification” bioRxiv [Preprint]. 2024 Aug. 26: 2024.08.26.609777. doi: 10.1101/2024.08.26.609777, the content of which is incorporated herein by reference in its entirety.

Disclosed herein include in vitro methods and compositions for single-cell proteomics analysis of mammalian embryos at various developmental stages and uses thereof to select blastomeres having a higher developmental potential and to detect perturbation-induced change in the protein composition of embryo cells. In some embodiments, a method for determining protein composition of an embryo in vitro is disclosed. The method can comprise culturing one or more embryos at the zygote stage in an embryo culture media until the one or more embryos reach at least early 2-cell blastomere stage, separating sister blastomeres of an embryo into single blastomeres, subjecting the single blastomeres to single-cell mass-spectrometry analysis to obtain a proteomic profile of each single blastomere, and identifying a first set of proteins differentially abundant in a first single blastomere and a second set of proteins differentially abundant in a second single blastomere, both blastomeres derived from a same embryo. The first single blastomere can be termed as alpha blastomere and the second single blastomere as beta blastomere.

Disclosed herein also includes a method for determining protein composition of an embryo at the zygote stage in vitro. The method can comprise splitting a zygote stage embryo into two zygote halves, subjecting the two zygote halves to single-cell mass-spectrometry analysis to obtain a proteomic profile of each zygote half, identifying a first set of proteins differentially abundant in a first zygote half and a second set of proteins differentially abundant in a second zygote half.

Disclosed herein also includes a method for detecting perturbation-induced change in embryo cells. The method can comprise introducing a perturbation to an embryo at the zygote stage, determining a protein composition of the embryo according to the method described herein, and detecting the perturbation-induced change on the protein composition of single blastomeres, optionally the determining comprises comparing the protein composition obtained in the presence of the perturbation with a protein composition obtained in the absence of the perturbation.

Disclosed herein also includes a method of selecting blastomeres having a higher developmental potential. In some embodiments, a method of selecting blastomeres from mammalian embryos comprises culturing one or more embryos at the zygote stage in an embryo culture media until the one or more embryo reaches 2-cell stage or 4-cell stage, separating sister blastomeres of an embryo into single blastomeres, detecting the abundance of one or more proteins selected from Nedd8, Gps1, PSMC4, or a combination thereof in the single blastomeres, and selecting one or more single blastomeres based on the abundance level of the one or more proteins selected from Nedd8, Gps1, PSMC4, or a combination thereof.

### Embryogenesis and Mammalian Development

Disclosed herein include methods and composition for single-cell proteomics analysis of a mammalian embryo in vitro and the uses thereof to select blastomeres and to assess the health status and developmental potential of an embryo. In some embodiments, the methods disclosed herein do not comprise any in vivo step. In some embodiments, none of the embryos, the 2-cell blastomeres, the 4-cell blastomeres, the 8-cell blastomeres, the ESCs, the TSCs, is present in an in vivo environment in any of the steps disclosed herein. The in vivo environment can comprise a tissue, or organ, an organism, or a combination thereof.

Mammalian embryogenesis is the process of cell division and cellular differentiation during early prenatal development which leads to the development of a mammalian embryo. While mammalian embryogenesis has some common features across all species, it will be appreciated that different mammalian species develop in different ways and at different rates. In general, though, the fertilized egg undergoes a number of cleavage steps (passing through two cell, four cell and eight cell stages) before undergoing compaction to form a solid ball of cells called a morula, in which the cells continue to divide. Ultimately the internal cells of the morula give rise to the inner cell mass and the outer cells to the trophectoderm. The morula in turn develops into the blastocyst, which is surrounded by trophectoderm and contains a fluid-filled vesicle, with the inner cell mass at one end.

The term “embryo” as used herein refers to a mammalian organism from the single cell stage. A developmental stage of an embryo can be defined by the development of specific structures and can be used to define equivalent stages in development of other species. In some embodiments, a developmental stage of an embryo can be defined according to “Carnegie stages”, which is a standardized system used to provide a unified developmental chronology of the vertebrate embryo.

In some embodiments, “Carnegie stages” have been established to describe stages of human development. Each stage is defined by the development of specific structures, and can be used to define equivalent stages in development of other species. The earliest Carnegie stages are as follows in Table 1:

TABLE 1
Carnegie Stages of Development

| Carnegie | Days since | |
|---|---|---|
| stage | ovulation (approx.) | Characteristic events/structures |
| 1 | 1 | fertilization; polar bodies |
| 2 | 2-3 | cleavage; morula; compaction |
| 3 | 4-5 | blastocyst and blastocoele; trophoblast and embryoblast |
| 4 | 6 | syncytiotrophoblast; cytotrophoblast; anchoring to |
| | | endometrium |
| 5(a) | 7-8 | implantation; embryonic disc; bilaminar germ disc; |
| | | primary yolk sac |
| 5(b) | 9-10 | formation of trophoblast lacunae; complete penetration |
| | | into endometrium; amniotic cavity; primary umbilical |
| | | vesicle |
| 5(c) | 11-16 | pre-chordal plate; extra- embryonic mesoblast; secondary |
| | | yolk sac |
| 6 | 17 | primitive streak, primitive node, primitive groove; |
| | | secondary umbilical vesicle; primordial germ cells; body |
| | | stalk |

In some embodiments, the mammalian embryos generated herein are mouse embryos. Theiler has established numbered stages of murine development. The earliest stages, as applied to (C57BLxCBA)F1 mice, are described in the “emouse digital atlas” (www.emouseatlas.org) as follows in Table 2.

TABLE 2
Theiler Stages

| Theiler | Dpc* | | Cell | |
|---|---|---|---|---|
| Stage | (range) | | number | (C57BL × CBA)Fl mice |
| 1 | 0-0.9 | (0-2.5) | 1 | One-cell egg |
| 2 | 1 | (1-2.5) | 2-4 | Dividing egg |
| 3 | 2 | (1-3.5) | 4-16 | Morula |
| | | | (or 8-16) | |
| 4 | 3 | (2-4) | 16-40 | Blastocyst inner cell mass apparent |
| | | | (or 16-32) | |
| 5 | 4 | (3-5.5) | | Blastocyst (zona-free) |
| 6 | 4.5 | (4-5.5) | | Attachment of blastocyst, primary endoderm covers |
| | | | | blastocoelic surface of inner cell mass |
| 7 | 5 | (4.5-6) | | Implantation and formation of egg cylinder, |
| | | | | Ectoplacental cone appears, enlarged epiblast, primary |
| | | | | endoderm lines mural trophectoderm |
| 8 | 6 | (5-6.5) | | Differentiation of egg cylinder, Implantation sites 2 × 3 |
| | | | | mm. Ectoplacental cone region invaded by maternal |
| | | | | blood, Reichert's membrane and proamniotic cavity |
| | | | | form |
| 9a | 6.5 | (6.25-7.25) | | Pre-streak(PS), advanced endometrial reaction, ecto |
| | | | | lacental cone invaded by blood, |
| | | | | extraembryonic ectoderm, embryonic axis visible |
| 9b | | | | Early streak(ES), gastrulation starts, first evidence of |
| | | | | mesoderm |
| 10a | 7 | (6.5-7.75) | | Mid streak (MS), amniotic fold starts to form |
| 10b | | | | Late streak, no bud (LSOB), exocoelom |
| 10c | | | | Late streak, early bud (LSEB), allantoic bud first |
| | | | | appears, node, amnion closing |
| 11a | 7.5 | (7.25-8) | | Neural plate (NP), head process developing, amnion |
| | | | | complete |
| 11b | | | | Late neural plate (LNP), elongated allantoic bud |
| 11c | | | | Early head fold (EDF) |
| 11d | | | | Late head fold (LHF), foregut invagination |
| 12a | 8 | (7.5-8.75) | | 1-4 somites, allantois extends, first branchial arch, |
| | | | | heart starts to form, foregut pocket visible, preotic |
| | | | | sulcus at 2-3 somite stage) |
| 12b | | | | 5-7 somites, allantois contacts chorion at the end of |
| | | | | TS12, Absent 2^{nd} arch, >7 somites |
| 13 | 8.5 | (8-9.25) | | Turning of the embryo, 1^{st} branchial arch has |
| | | | | maxillary and mandibular components, 2^{nd} arch present; |
| | | | | Absent 3rd branchial arch |
| 14 | 9 | (8.5-9.75) | | Formation & closure of ant. neuropore, otic pit |
| | | | | indented but not closed, 3^{rd} branchial arch visible; |
| | | | | Absent forelimb bud |
| 15 | 9.5 | (9-10.5) | | Formation of post. neuropore, forelimb bud, |
| | | | | forebrain vesicle subdivides; Absent hindlimb bud, |
| | | | | Rathke's pouch |
| 16 | 10 | (9.5-10.75) | | Posterior neuropore closes, Formation of hindlimb & |
| | | | | tail buds, lens plate, Rathke's pouch; the indented nasal |
| | | | | processes start to form· Absent thin & long tail |
| 17 | 10.5 | (10-11.25) | | Deep lens indentation, adv. <level of brain tube, tail |
| | | | | elongates and thins, umbilical hernia starts to form; |
| | | | | Absent nasal pits |
| 18 | 11 | (10.5-11.25) | | Closure of lens vesicle, nasal pits, cervical somites no |
| | | | | longer visible; Absent auditory hillocks, anterior |
| | | | | footplate |
| 19 | 11.5 | (11-12.25) | | Lens vesicle completely separated from the surface |
| | | | | epithelium, Anterior, but no posterior, footplate. |
| | | | | Auditory hillocks first visible; Absent retinal |
| | | | | pigmentation and sign of fingers |
| 20 | 12 | (11.5-13) | | Earliest sign of fingers, (splayed out), posterior |
| | | | | footplate apparent, retina pigmentation apparent, tongue |
| | | | | well-defined, brain vesicles clear; Absent 5 rows of |
| | | | | whiskers, indented |
| 21 | 13 | (12.5-14) | | Anterior footplate indented, elbow and wrist |
| | | | | identifiable, 5 rows of whiskers, umbilical hernia now |
| | | | | clearly apparent; Absent hair follicles, fingers separate |
| | | | | distally |
| 22 | 14 | (13.5-15) | | Fingers separate distally, only indentations between |
| | | | | digits of the posterior footplate, long bones of limbs |
| | | | | present, hair follicles in pectoral, pelvic and trunk |
| | | | | regions; Absent open eyelids, hair follicles in cephalic |
| | | | | region |

| 23 | 15 | Fingers & Toes separate, hair follicles also in cephalic |
|---|---|---|
| | | region but not at periphery of vibrissae, eyelids open; |
| | | Absent nail primordia, fingers 2-5 parallel |
| 24 | 16 | Reposition of umbilical hernia, eyelids closing, fingers |
| | | 2-5 are parallel, nail primordia visible on Toes; Absent |
| | | wrinkled skin, fingers & toes joined together |
| 25 | 17 | Skin is wrinkled, eyelids are closed, umbilical hernia is |
| | | gone; Absent ear extending over auditory meatus, long |
| | | whiskers |
| 26 | 18 | Long whiskers, eyes barely visible through closed |
| | | eyelids, ear covers auditory meatus |
| 27 | 19 | Newborn Mouse |

*“dpc” indicates days post conception, with the morning after the vaginal plug is found being designated 0.5 d or E0.5.

The methods and compositions herein described can be applied to embryos from any suitable mammalian species, such as: primates, including humans, great apes (e.g., gorillas, chimpanzees, orangutans), old world monkeys, new world monkeys; rodents (e.g., mice, rats, guinea, pigs, hamsters); cats; dogs; lagomorphs (e.g., rabbits); cows; sheep; goats; horses; pigs; and any other livestock, agricultural, laboratory or domestic mammals. The methods and compositions herein described can be applied to an embryo from any non-human mammal, including but not limited to those described above.

In some embodiments, the mammalian embryos generated herein are human embryos. Human embryonic development is characterized by the processes of cell division and cellular differentiation of the embryo that occurs during the early stages of the development. A germinal stage of a human embryonic development refers to the time from fertilization through the development of the early embryo until implantation is completed in the uterus. The germinal stage takes about 10 days. During this stage, the one-celled zygote divides in a process referred to as cleavage. A blastocyst is then formed and implants in the uterus. Embryogenesis continues with the next stage of gastrulation, when the three germ layers of the embryo form in a process referred to as histogenesis, and the processes of neurulation and organogenesis follow.

In particular, following fertilization, the resulting one-celled zygote undergoes multiple mitotic cleavages, a series of mitotic divisions that occur after fertilization to create a multicellular embryo, resulting in the production of blastomeres (i.e., the dividing cells). The cleavage/cell division goes through a two-cell stage (approximately day one of cleavage), four-cell stage (approximately day two of cleavage), eight-cell stage (approximately day three of cleavage), and sixteen-cell stage (approximately day four of cleavage). The two-cell stage embryo comprises two blastomeres, the four-cell stage embryo comprises four blastomeres, the eight-cell stage embryo comprises eight blastomeres, and the sixteen-cell stage embryo comprises sixteen blastomeres, and so on. Initially, the dividing cells or blastomeres are undifferentiated and aggregated into a sphere enclosed within the zona pellucida of the embryo. When eight blastomeres have formed (8-cell stage), the cells start to compact and develop gap junctions, enabling them to develop in an integrated way and co-ordinate their response to physiological signals and environmental cues. A morula appears approximately four days after fertilization and refers to the solid sphere of cells within the zona pellucida when the cells number reaches sixteen (16-cell stage). Medically, this is often known as the final stage before the formation of a fluid-filled blastocoel cavity, which precedes blastula formation. Recent time-lapse microscopy observations suggest that compaction may represent an important checkpoint for human embryo viability, through which chromosomally abnormal blastomeres are sensed and eliminated by the embryo. Compaction is critical because it sets anatomical differences between cells (inner versus outer), ultimately determining their fate. The group of cells present in the center of the morula will eventually give rise to the inner cell mass and the embryo proper. The cells at the periphery, the outer cell mass cells, are critical in the cavitation of the morula that occurs as it transitions into a blastocyst.

Cleavage is the first stage in blastulation, the process of forming the blastocyst. A blastocyst refers to an embryo at the blastocyst stage. The term “blastocyst stage” as used herein refers to an early embryonic development stage that occurs around 5-6 days after fertilization and is characterized by a ball of cell containing about 50-150 cells and two distinct cell types of inner cell mass (ICM) and trophectoderm (TE) surrounded by a membrane called the zona pellucida. The ICM, also referred to as embryoblast, refers to a mass of cells inside the blastocyst that will eventually give rise to the definitive structures of the fetus. The ICM is surrounded by a single layer of trophoblast cells of the TE. The trophoblast cells form the outer layer of the blastocyst and line the inner side of the zona pellucida. Trophoblast cells are present four days after fertilization in humans and provide nutrients to the embryo and develop into a large part of the placenta.

The ICM and the TE will generate distinctly different cell types as implantation starts and embryogenesis continues. Trophectoderm cells form extraembryonic tissues, which act in a supporting role for the embryo proper. Furthermore, these cells pump fluid into the interior of the blastocyst, causing the formation of a polarized blastocyst with the ICM attached to the trophectoderm at one end. This polarization leaves a cavity, the blastocoel, creating the blastocyst structure. Accordingly, a blastocyst formation is characterized by the fluid-filled blastocoele, the ICM, and the fully differentiated trophectoderm-derived trophoblast. This difference in cellular localization causes the ICM cells exposed to the fluid cavity to adopt a primitive endoderm (or hypoblast) fate, while the remaining cells adopt a primitive ectoderm (or epiblast) fate. The hypoblast contributes to extraembryonic membranes and the epiblast will give rise to the ultimate embryo proper as well as some extraembryonic tissues. In some embodiments, the ICM can be used to predict the quality of an embryo during in vitro fertilization (IVF). The ICM's morphology is also a strong predictor of live birth after a frozen-thawed single embryo transfer.

The methods and compositions described herein can be used to assess the protein composition as well as the developmental potential of embryo's cells of an embryo in a pre-implantation stage. The term “pre-implantation stage” can be used herein to refer to a stage of development earlier than the stage corresponding to Theiler stage 7, Carnegie stage 5(a), and corresponding stages in other species.

In some embodiments, the methods and compositions described herein are used to determine single-cell proteomics profile of blastomeres at two-cell stage (two blastomeres), including early two-cell stage, mid two-cell stage, and late two-cell stage. In some embodiments, the methods and compositions described herein are used to determine single-cell proteomics profile of blastomeres at four-cell stage (four blastomeres) or at eight-cell stage. In some embodiments, the methods and composition described herein are used to determine single-cell proteomics profiles of embryos prior to a blastocyst stage. In some embodiments, the methods and compositions described herein are used to determine single-cell proteomics profile of embryos at the zygote stage.

### Single-Cell Proteomics Analysis of Mammalian Embryos

Provided herein include in vitro methods for single-cell proteomics analysis of mammalian embryos at different development stages (e.g., zygote, two-cell stage, four-cell stage, eight-cell stage, or beyond). In some embodiments, a method for determining protein composition of an embryo in vitro comprises culturing one or more embryos at the zygote stage in an embryo culture media until the one or more embryos reach at least early two-cell blastomere stage, separating sister blastomeres of an embryo into single blastomeres, subjecting the single blastomeres to single-cell mass-spectrometry analysis to obtain a proteomic profile of each single blastomere, and identifying a first set of proteins differentially abundant in a first single blastomere and a second set of proteins differentially abundant in a second single blastomere, both blastomeres derived from a same embryo. The first set of proteins differentially abundant in a first single blastomere are different from the second set of proteins differentially abundant in the second single blastomere. The first single blastomere can be termed alpha blastomere and the second single blastomere can be termed beta blastomere. Accordingly, the first set of proteins differentially abundant in the alpha blastomere can be termed as alpha proteins, and the second set of proteins differentially abundant in the beta blastomere can be termed as beta proteins. The protein asymmetry demonstrated in mammalian embryo is correlated with their developmental potential. In some embodiments, beta blastomeres have a higher developmental potential and can give rise to a blastocyst with more epiblast cells than alpha blastomeres. Accordingly the proteome asymmetry of mammalian embryo can be used to predict the developmental potential of blastomeres.

In some embodiments, the method further comprises identifying differentially abundant proteins in a third single blastomere and a fourth blastomere (e.g., in a four-cell stage embryo). For example, the method can comprise identifying the first set of proteins differentially abundant in the third single blastomere and a second set of proteins differentially abundant in the fourth single blastomere, wherein the first, second, third and fourth blastomeres are sister blastomeres derived from a same embryo. In some embodiments, a two-cell embryo can comprise an alpha blastomere and a beta blastomere. In some embodiments, a four-cell embryo can comprise two alpha blastomeres and two beta blastomeres. The alpha and beta identity of blastomeres can vary in a different degree. Some blastomeres can have a strong alpha or beta identity while others can have a relatively weak alpha or beta identity.

The method can comprise culturing a mammalian embryo at the zygote stage in an embryo culture media until the embryo forms an early 2-cell stage embryo comprising two blastomeres, as a result of zygotic division. In some embodiments, the zygote is a zygote having two pronucleic, also referred to a 2PN zygote. In some embodiments, the mammalian embryo is a mouse embryo, and the embryo at the zygote state is cultured in the embryo culture media for about 1-3 hours to reach the early 2-cell stage. In some embodiments, the mouse embryo can be cultured in the embryo culture media for a certain time period to reach a late 2-cell stage, optionally for about 17-19 hours. In some embodiments, the mouse embryo is cultured in the embryo culture media for a period of time to reach a 4-cell stage (e.g., 26-28 hours). In some embodiments, the mammalian embryo is a human zygote. The human zygote can be cultured in an embryo culture media for about 6-12 hours. In some embodiments, the human zygote is cultured in an embryo culture media for about 12-20 hours until the completion of the first cleavage division. In some embodiments, one or more embryos or separated blastomeres are cultured in the embryo culture media for a suitable time period and condition allowing the one or more embryos or blastomeres to reach an 8-cell blastomere stage or a blastocyst stage.

The embryo culture media can comprise a basal culture medium. The basal culture medium may comprise water, amino acids, physiological salts, energy substrates such as a carbon source, lipids, and a buffer. Suitable carbon sources may be assessed by one of skill in the art from compounds such as glucose, sucrose, sorbitol, galactose, mannose, fructose, mannitol, maltodextrin, trehalose dihydrate, and cyclodextrin. Basal media are commercially available, for example, under the trade names Advanced DMEM/Fl2 (Gibco, 12634-010) and CMRL-1066 (Invitrogen or Sigma). In some embodiments, the carbon source comprises glucose. The embryo culture media can comprise a pH buffer such as biocarbonate or HEPES. Amino acids can comprise essential amino acids and non-essential amino acids. Exemplary essential amino acids can include valine, leucine, methionine, phenylalanine, tryptophan, threonine, histidine, and lysine. Exemplary non-essential amino acids can include L-glycine, L-alanine, L-asparagine, L-aspartic acid, L-glutamic acid, L-proline and L-serine. The embryo culture media can further comprise an antibiotic. The antibiotic can comprise Penicillin-streptomycin, Amphotericin B, Ampicillin, Erythromycin, Gentamycin, Kanamycin, Neomycin, Nystatin, Polymyxin B, Tetracycline, Thiabendazole, Tylosin, or any combination thereof. In some embodiments, the embryo culture media can comprise physiological salts, glucose, pH buffer (biocarbonate or HEPES), essential amino acids, non-essential amino acids, glutamine dipeptide, EDTA, gentamicin and water. In some embodiments, the embryo culture media used herein are designed for Day 1-6 mammalian embryo culture and transfer.

The embryo culture media may be free, substantially free, or essentially free of proteins. In some embodiments, the embryo culture media is not protein free and comprises a non-human serum or serum substitute. The non-human serum or serum substitute can comprise fetal bovine serum, bovine serum albumin, rat serum, KnockOut™ Serum Replacement, human serum albumin, or any combination thereof. In some embodiments, the embryo culture media can further comprise human α- and β-globulins. In some embodiments, the embryo culture media comprises a total protein concentration of about 10 mg/ml. In an exemplary embodiment, the embryo culture media can comprise human serum albumin, human α- and β-globulins, calcium chloride, sodium chloride, potassium chloride, potassium phosphate, magnesium sulfate, sodium bicarbonate, glucose, lactate Na salt, sodium pyruvate, amino acids, glycyl-glutamine, EDTA, gentamicin, water, or any combination thereof.

Sister blastomeres of an embryo at different stages of development, including a 2-cell stage (early or late two-cell stage) or a four-cell stage, can be separated into single blastomeres. For example, the two blastomeres of an embryo at a two-cell stage can be separated into two single blastomeres. The four blastomeres of an embryo at a four-cell stage can be separated into four single blastomeres. Separating blastomeres of an embryo into single blastomeres can comprise removing the zona pellucida of the embryo by, for example, placing the embryo in an acidic solution (e.g., acidic Tyrode's solution treatment) for a suitable time period to ensure only the zona pellucida dissolves, not the underlying cells. Once the zona pellucida dissolves, the embryo can then be bisected or split into single blastomeres, for example, using capillary. The method can further comprise washing the single blastomeres following the bisection. Embryo bisecting or splitting can be performed at different stages of development, such as the two-cell, four-cell, or eight-cell stage. In some embodiment, a zygote-stage embryo can be split into two zygote halves. For example, the splitting can be meridionally in alignment with the animal-vegetal axis as defined by the position of the polar body. Alternatively, the splitting can be equatorial, i.e., perpendicular to animal-vegetal axis.

The method can further comprise subjecting the single blastomeres to mass-spectrometry analysis to obtain a proteomic profile of each single blastomere. As used herein, the term “mass spectrometry” refers to an analytical tool able to volatilize/ionize analytes to form gas-phase ion and determine their absolute or relative molecular masses. Typically, mass spectrometers can be used to identify unknown compounds via molecular weight determination, to quantify known compounds, and to determine structure and chemical properties of molecules. Suitable methods of volatilization/ionization are matrix-assisted laser desorption ionization (MALDI), electrospray, laser/light, thermal, electrical, atomized/sprayed and the like, or combinations thereof. Suitable forms of mass spectrometry include, but are not limited to, ion trap instruments, quadrupole instruments, electrostatic and magnetic sector instruments, time of flight instruments, time of flight tandem mass spectrometer (TOF MS/MS), Fourier-transform mass spectrometers, Orbitraps and hybrid instruments composed of various combinations of these types of mass analyzers. These instruments can, in turn, be interfaced with a variety of other instruments that fractionate the samples (for example, liquid chromatography or solid-phase adsorption techniques based on chemical, or biological properties) and that ionize the samples for introduction into the mass spectrometer, including matrix-assisted laser desorption (MALDI), electrospray, or nanospray ionization (ESI) or combinations thereof.

In some embodiments, the mass spectrometry assays, instruments and systems suitable for single-cell analysis can include, but are not limited to, matrix-assisted laser desorption/ionisation time-of-flight (MALDI-TOF) mass spectrometry, electrospray ionization mass spectrometry (ESI-MS), ESI quadrupole orthogonal TOF (ESI-QTOF) mass spectrometry, ESI Fourier transform mass spectrometry (ESI-FTMS), ion trap mass spectrometry, triple quadrupole mass spectrometry (TQMS), ion mobility spectrometry-mass spectrometry (IMS-MS), Fourier transform ion cyclotron resonance mass spectrometry (FT-ICR MS), surface-enhanced laser desorption/ionization time-of-flight mass spectrometry (SELDI-TOF MS), desorption/ionization on silicon mass spectrometry (DIOS-MS), secondary ion mass spectrometry (SIMS), atmospheric pressure chemical ionization mass spectrometry (APCI-MS), atmospheric pressure photoionization mass spectrometry (APPI MS), gas chromatography-mass spectrometry (GC-MS), liquid chromatograph-mass spectrometry (LC-MS), inductively couples plasma mass spectrometry (ICP-MS), and tandem mass spectrometry (MS/MS).

Prior to mass-spectrometry analysis, the single blastomeres or zygote halves are lysed, and the cell lysate can be contacted with a trypsin enzyme to digest proteins into smaller peptides. Any suitable cell lysis methods can be used for lysing the single blastomeres or zygote halves. The cell lysis method can be a physical method or a chemical method. Exemplary cell lysis methods include, but are not limited to, sonication, bead beating, high-pressure homogenization, thermal shock, freeze-thaw cycles, manual grinding, detergent-based lysis, enzymatic lysis, osmotic shock, and alkaline lysis. In some embodiments, the cell lysis method used herein is a physical or mechanical lysis, therefore eliminating cleanup-related losses. For example, the single blastomeres or zygote halves can be lysed in a heating-cooling process. In an exemplary embodiment, the single blastomeres or zygote halves can be heated in a thermocycler to 90° C. for 10 mins and then cooled down to 12° C.

In some embodiments, the mass spectrometry technique used here is single-cell mass spectrometry. Single-cell mass spectrometry is a MS technique allowing for the analysis of individual cells to understand their unique composition and characteristics at the molecular level. It enables the identification and quantification of various biomolecules, including proteins, metabolites, and lipids, within a single cell. This technique is particularly useful for studying cellular heterogeneity and understanding the complex biological processes that occur at the single-cell level. The single-cell MS techniques used herein can minimize sample loss and excessive sample dilution while maintaining a high throughput measurement. In some embodiments, the sample preparation used in single-cell MS employs a freeze-heat cycle to lyse cells and exclusively utilizes chemicals compatible with MS analysis, eliminating the need for sample cleanup.

In some embodiments, the methods described herein apply multiplexed LC-ESI-MS/MS or LC-MS/MS to quantify proteins from single cells (e.g., single blastomeres or zygote halves). In some embodiments, the MS method used herein is Single Cell ProtEomics by Mass Spectrometry (SCoPE-MS) or SCoPE2-MS. Detailed description about SCoPE-MS and SCoPE2-MS can be found in the related publications including, for example, Specht, H., Emmott, E., Petelski, A. A. et al. Single-cell proteomic and transcriptomic analysis of macrophage heterogeneity using SCoPE2. Genome Biol 22, 50 (2021), and Budnik, B., Levy, E., Harmange, G. et al. SCoPE-MS: mass spectrometry of single mammalian cells quantifies proteome heterogeneity during cell differentiation. Genome Biol 19, 161 (2018) the contents of which are incorporated here by references in their entireties. Briefly, the method resolves two major challenges by delivering the proteome of a mammalian cell to a MS instrument with minimal protein losses and simultaneously identifying and quantifying peptides from single-cell samples. In some embodiments, single blastomeres or zygote halves are lysed mechanically to obviate chemicals that may undermine peptide separation and ionization or sample cleanup that may incur significant losses. In some embodiments, the data is analyzed using ScoPE2 pipeline available at zenodo.org/record/4339954#.YnHcYSfMLOQ.

In some embodiments, the MS method described herein use tandem mass tags (TMTs) which are isobaric tags used in mass spectrometry to enable the relative quantification of multiple samples in a single experiment. As a person skilled in the art would understand, each TMT tag has the same overall mass, making them indistinguishable in the first mass spectrometry stage (MS1). Peptides from each single cell are labeled with isobaric TMT, and therefore labeled peptides with the same sequence (and thus same mass) appear as a single mass/charge cluster in the MS1 scan. During MS/MS, in addition to generating peptide fragments which facilitate peptide identification, fragmentation generates reporter ion (RI), whose abundances reflect protein abundances in the corresponding samples (single cells). This method is suitable for multiplexing and usually employed for cost-effective increase in throughput. TMT allows quantifying the level of each TMT-labeled peptide in each sample while identifying its sequence from the total peptide amount pooled across all samples. In some embodiments, MS (e.g., SCoPE-MS) capitalizes on this capability by augmenting each single-cell set with a sample comprised of about 200 carrier cells that provide enough ions for peptide sequence identification. The carrier cells also help reduce losses from single cells, since most of the peptides lost due to surface adhesion will likely originate from the carrier cells.

Accordingly, in some embodiments, the mass-spectrometry analysis comprises preparing an isobaric carrier. In some embodiments, mammalian embryonic stem cells are used as an isobaric carrier sample. Preparing the isobaric carrier can comprise lysing embryonic stem cells (ESCs), contacting the cell lysate with a trypsin enzyme to digest proteins into smaller peptides, and labeling the cell digest with TMT. The method can also comprise culturing the embryonic stem cells in a suitable culture medium. In some embodiments, the ESCs are mouse ESCs. In some embodiments, the ESCs are human ESCs. The ESCs can be cultured in a suitable culture media suitable for stem cell and pluripotent stem cell proliferation as will be understood by a person skilled in the art. For example, the ESCs can be cultured in a culture medium free of serum or substantially free of serum or essentially free of serum. The culture medium may comprise a serum replacement medium. Such serum replacement media are commercially available under the trade names KSR (KnockOut™ Serum Replacement, Invitrogen, 10828-010) and N2B27 (e.g., Invitrogen, ME100137L1). The serum replacement medium may be included in the culture medium at about 5% to about 60%, about 10% to about 50%, about 15% to about 45%, or about 20% to about 40%. The culture medium may comprise growth factors such as a fibroblast growth factor (FGF) and/or transforming growth factor β (TGF β).

In some embodiments, the MS method used herein is label-free. In some embodiments, the MS methods used herein does not require isobaric labeling. Accordingly, in some embodiments, the peptides in the sample are not labeled with TMTs. Each sample containing peptides obtained from a single blastomere or a zygote halve are individually analyzed using MS (e.g., LC-MS/MS).

In some embodiments, the single-cell proteomics method uses label-free data-independent acquisition (DIA) for data acquisition. Label-free DIA in mass spectrometry is a quantitative technique that identifies and measures the relative abundance of proteins without using stable isotope tags. This approach relies on directly comparing the abundance of peptides (or their fragment ions) in different samples to determine relative protein abundances. In some embodiments, SCoPE2 employs DIA which systematically samples all ions within a defined mass range, leading to increased sensitivity and proteome coverage. In some embodiments, the single-cell proteomics method uses labeled data-dependent acquisition (DDA) for data acquisition. In contrast to DIA that fragments and analyzes all peptides during the second stage of tandem MS, the DDA mass spectrometer selects and fragments a subset of ion based on their abundance in the initial MS survey scan (MS1). The fragment ion data of the selected ions is then analyzed to identify and quantify the peptides and proteins in the sample.

In some embodiments, the method identifies differentially abundant proteins in single blastomeres derived from a same embryo at various development stages (e.g., early two-cell stage, late two-cell stage, four-cell stage, or eight-cell stage, and so on). For example, the method can comprise identifying a first set of proteins differentially enriched in a first single blastomere and a second set of proteins differentially enriched in a second single blastomere, both blastomeres derived from a same embryo. In some embodiments, the blastomeres derived from a same embryo exhibit protein asymmetry, i.e., the first set of proteins and the second set of proteins are different. The first single blastomere can be termed as alpha blastomere, and the second single blastomere can be termed as beta blastomere. Accordingly, the first set of proteins differentially enriched in the first single blastomere can be termed as alpha proteins, and the second set of proteins differentially enriched in the second single blastomere can be termed as beta proteins. In some embodiments, a two-cell embryo can comprise an alpha blastomere and a beta blastomere. A four-cell embryo can comprise two alpha blastomeres and two beta blastomeres. In some embodiments, the method further comprises identifying differentially abundant proteins in a third single blastomere and a fourth blastomere (e.g., in a four-cell stage embryo).

In some embodiments, the method further comprises determining the degree of asymmetry in the single blastomeres. The degree of asymmetry can be defined as the ratio of the mean abundance of the first set of proteins differentially enriched in the first single blastomere (alpha proteins) and that of the second set of proteins differentially enriched in the second single blastomere (beta proteins). In some embodiments, the degree of asymmetry for each blastomere can be the fold change between the mean abundances of alpha proteins and beta proteins in each blastomere. The degree of asymmetry can also be used to indicate the strength of alpha and beta polarization. The degree of asymmetry may vary for blastomeres at the same developmental stage. For example, a four-cell embryo can comprise two relatively strong alpha blastomeres and two relatively strong beta blastomeres. In another example, a four-cell embryo can comprise one strong alpha blastomere, a weak beta blastomere, and two strong beta blastomeres. In some embodiments, the degree of asymmetry of a blastomere is calculated as a logarithm of the ratio of the mean abundances of the alpha proteins and the beta proteins in the blastomere (see, for example, FIG. 7, panel e). A positive value indicates the blastomere is an alpha blastomere, and a negative value indicates the blastomere is a beta blastomere. The absolute value indicates the degree of polarization: a higher absolute value indicates a more polarized blastomere. The degrees of asymmetry of single blastomeres derived from embryos may also vary across different development stages. In some embodiments, the method can further comprises comparing the degrees of asymmetry of single blastomeres derived from embryos across different developmental stages. In some embodiments, the degree of asymmetry can increase from the early to the late two-cell stage or from the two-cell stage to the four-cell stage.

The method can further comprise identifying biological processes that are differential among the single blastomeres derived from a same embryo. For example, ubiquitin- and autophagy-related terms are enriched in beta blastomeres, whereas proteasome-related terms are more enriched in alpha blastomeres. Protein transport terms (channel and signaling-related, molecular motors, and vesicle transport) are enriched in beta blastomeres, with processes related to molecular motors exhibiting the highest median fold difference.

In some embodiments, the beta proteins comprise proteins that impact the trophectoderm and epiblast lineage such as proteins supporting the epiblast formation and suppressing the trophectoderm formation. In some embodiments, the beta proteins comprise Nedd8 and Gps1, and the alpha proteins comprise PSMC4. In some embodiments, knockdown of one of the beta proteins such as Nedd8 can significantly increase trophectoderm cells relative to control, but does not have a significant effect on the epiblast or primitive endoderm, suggesting that some of the beta proteins can inhibit the specification and/or proliferation of trophectoderm cells. In some embodiments, knockdown of one of the beta proteins such as Gsp1 can reduce the portion of epiblast cells with a less significant reduction in primitive endoderm and no significant reduction in trophectoderm, suggesting that some of the beta proteins play a role in promoting pluripotency.

The present disclosure demonstrates that the alpha-beta identity of a blastomere correlates with differences in developmental potential, with beta blastomeres having a higher developmental potential and alpha blastomeres having a lower developmental potential. In some embodiments, blastomeres with a higher beta identify give rise to blastocysts with a higher proportion of epiblast cells. In some embodiments, beta blastomeres can give rise to a blastocyst with more epiblast cells than alpha blastomeres. In some embodiments, beta blastomeres are more likely to give rise to blastocysts with four epiblast cells, the minimum number required for successful further development. Accordingly the proteome asymmetry of mammalian embryo can be used to predict the developmental potential of blastomeres.

Disclosed herein also includes a method of selecting blastomeres having a higher developmental potential. In some embodiments, the method of selecting blastomeres from mammalian embryos comprises culturing one or more embryos at the zygote stage in an embryo culture media until the one or more embryo reaches 2-cell stage or 4-cell stage, separating sister blastomeres of an embryo into single blastomeres, detecting the abundance of one or more proteins selected from Nedd8, Gps1, PSMC4, or a combination thereof in the single blastomeres, and selecting one or more single blastomeres based on the abundance level of the one or more proteins selected from Nedd8, Gps1, PSMC4, or a combination thereof. In some embodiments, the selecting comprises comparing the abundance of the one or more proteins selected from Nedd8, Gps1, PSMC4, or a combination thereof with a reference level. The reference level can be the abundance of the corresponding protein(s) measured from a control beta blastomere or a control blastomere having a higher beta identity. The beta identity degree of the control blastomere can be obtained by calculating the fold change between the mean abundances of alpha proteins and beta proteins in the control blastomere as described above and in the Examples (e.g., FIG. 7 and Examples 2 and 7). In some embodiments, the one or more proteins are beta proteins (e.g., Nedd8 or Gps1). In some embodiments, the one or more proteins comprise alpha protein (e.g., PSMC4).

In some embodiments, one blastomere of the sister blastomeres (e.g., from 2-cell or 4-cell stage) is labeled with a detectable lineage marker. The blastomere of the embryo can be labeled, for example, by injecting the blastomere with an mRNA encoding the detectable lineage tracing marker. For example, the blastomere can be injected with an mRNA encoding a detectable lineage tracking marker that encodes a membrane targeting sequence.

In some embodiments, the embryos described herein are mammalian embryos. In some embodiments, the mammalian embryos are non-human embryos, such as mouse embryos or rabbit embryos. In some embodiments, the mammalian embryos are human embryos.

Embryonic stages of the synthetic embryos described herein can be assessed compared to an in vivo or natural embryo counterpart at the same developmental stage by multiple ways including, but not limited to, morphology, length, weight, weight, expression of developmental marker genes using specific antibodies or primers, transcriptional profiling and the like, as further described herein below and in the Examples section. Morphology assessment of embryonic development can be performed by previously established morphological features such as described in Carnegie stages of development (Also, See, Table 1; Developmental stages in human embryos. R. O'Rahilly and F. Muller (eds), Carnegie Institution of Washington, Washington, DC, 1987), in Theiler stages of development (see, for example, Table 2; www. emouseatlas org) or according to embryonic days.

The method can further comprise identifying cells expressing the detectable lineage marker in each embryo (e.g., blastocyst). The activity or level of a lineage marker protein can be detected and/or quantified by detecting or quantifying the expressed marker polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like.

The differentially abundant proteins (e.g., alpha and beta proteins) identified herein can be used as a protein marker to predict the developmental potential and lineage fate of blastomeres. In some embodiments, one or more protein markers as described herein can be detected using any suitable method identifiable to a person skilled in the art. Numerous methods exist in the art for detecting the presence, absence, or amount of a marker gene product (e.g., mRNA and/or protein), as well as its localization in an embryo structure or subcellular localization (e.g., nucleus and/or cytoplasm). Marker expression may be assessed by any of a wide variety of well-known methods for detecting expression of a transcribed molecule or a protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification and sequencing methods.

In some embodiments, activity of a particular gene is characterized by a measure of gene transcript (e g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In some embodiments, detecting or determining expression levels of a marker and functionally similar homologs thereof, including a fragment or genetic alteration thereof (e.g., in regulatory or promoter regions thereof) comprises detecting or determining RNA levels for the marker of interest. In some embodiments, one or more cells from the synthetic embryo structure can be obtained and RNA is isolated from the cells. In some embodiments, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated. It is also possible to obtain cells from, e.g., the synthetic embryo cells and culture the cells in vitro, such as to obtain a larger population of cells from which RNA can be extracted. Methods for establishing cultures of non-transformed cells, i.e., primary cell cultures, are known in the art. In some embodiments, cells can be dissociated (e.g., by enzymatic or mechanical means), and isolated by methods known in the art (e g., Fluorescence-Activated Cell Sorting, Microfluidics, etc.)

When isolating RNA from, e.g., synthetic embryos at various developmental stages and/or cells comprising said synthetic embryos, it may be important to prevent any further changes in gene expression after the tissue or cells has been removed from the subject. Changes in expression levels are known to change rapidly following perturbations, e g., heat shock or activation with lipopolysaccharide (LPS) or other reagents. In addition, the RNA in the tissue and cells may quickly become degraded. Accordingly, in some embodiments, the tissue or cells obtained from a subject is snap frozen as soon as possible.

RNA can be extracted from cells by a variety of methods, e.g., the guanidium thiocyanate lysis followed by CsCl centrifugation. Methods for obtaining RNA from single-cells are also known in the art. The RNA sample can then be enriched in particular species. In some embodiments, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.). In some embodiments, the RNA population is enriched in marker sequences. Enrichment can be undertaken, e.g., by primer-specific cDNA synthesis, or multiple rounds of linear amplification based on cDNA synthesis and template-directed in vitro transcription.

The population of RNA, enriched or not in particular species or sequences, can further be amplified. As defined herein, an “amplification process” increases the number of copies of a polynucleotide (e.g., RNA). For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the disclosed methods to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used. Other known amplification methods which can be utilized herein include but are not limited to the so-called “NASBA” or “3 SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e g., Kwoh et al., Proc. Natl. Acad. Sci. USA 86, 1173 (1989)). Many techniques are known in the state of the art for determining absolute and relative levels of gene expression, commonly used techniques suitable for use in the disclosed methods include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a sample, washed, cleaved with RNase and exposed to a sensitive emulsion for autoradiography. The samples may be stained with hematoxylin to demonstrate the histological composition of the sample, and dark field imaging with a suitable light filter shows the developed emulsion. Nonradioactive labels such as digoxigenin may also be used. In some embodiments, the probe is labeled with a fluorescence moiety.

Alternatively, mRNA expression can be detected on a DNA array, chip or a microarray. Labeled nucleic acids of a test sample obtained from a subject may be hybridized to a solid surface comprising marker DNA. Positive hybridization signal is obtained with the sample containing marker transcripts. Methods of preparing DNA arrays and their use are well known in the art (see, e.g., U.S. Pat. Nos. 66,186,796; 6,379,897; 6,664,377; 6,451,536; 548,257; U.S. 20030157485). Serial Analysis of Gene Expression (SAGE) can also be performed (See for example U.S. Patent Application 20030215858). In some embodiments, next generation sequencing (e.g., RNA-seq) can be used to analyze total mRNA expression from one (e.g., single-cell RNA-seq) or more cells. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others. Methods for constructing sequencing libraries are known in the art.

The single cell sequencing can be high-throughput single cell RNA sequencing. In certain embodiments, the single cell sequencing is a low cost high-throughput single cell RNA sequencing. Not being bound by any particular theory, the single cell RNA sequencing is capable of efficiently and cost effectively sequencing thousands to tens of thousands of single cells. In certain embodiments, single cell RNA sequencing comprises pairing single cells in droplets with oligonucleotides for reverse transcription, wherein the oligonucleotides are configured to provide cell-of-origin specific barcodes uniquely identifying transcripts from each cell and a unique molecular identifier (UMI) uniquely identifying each transcript. In certain embodiments, single cell RNA sequencing comprises pairing single cells in droplets with single microparticle beads coated with oligonucleotides for reverse transcription, wherein the oligonucleotides contain a bead-specific barcode uniquely identifying each bead and a unique molecular identifier (UMI) uniquely identifying each primer. In some aspects of the disclosure, unbiased classifying of cells in a biological sample comprises sequencing the transcriptomes of thousands of cells, preferably tens of thousands of cells (e.g., greater than 1000 cells, or greater than 10,000 cells).

The activity or level of a lineage marker protein can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binderligand assays, immunohistochemical techniques, agglutination, complement assays, high performance liquid chromatography (HPLC), thin layer chromatography (TLC), hyperdiffusion chromatography, and the like.

Described below are non-limiting examples of techniques that may be used to detect marker protein according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-marker protein antibodies (unlabeled) are then brought into contact with the support and assayed by a secondary immunological reagent, such as labeled protein A or anti-immunoglobulin (suitable labels including ¹²⁵I, horseradish peroxidase, alkaline phosphatase, fluorophore). Chromatographic detection may also be used.

Immunohistochemistry may be used to detect expression of marker protein. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-marker protein antibodies, such as intrabodies, may also be used for imaging purposes, for example, to detect the presence of marker protein in cells or, e.g., an EP structure. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies that may be used to detect marker protein include any antibody, whether natural or synthetic, full length or a fragment thereof, monoclonal or polyclonal, that binds sufficiently strongly and specifically to the marker protein to be detected. An antibody may have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to the marker protein relative to other proteins, such as related proteins.

Antibodies are commercially available or can be prepared by methods known in the art. Antibodies and derivatives thereof that may be used encompass polyclonal or monoclonal antibodies, chimeric, human, humanized, primatized (CDR-grafted), veneered or single-chain antibodies as well as functional fragments, i.e., marker protein binding fragments, of antibodies. For example, antibody fragments capable of binding to a marker protein or portions thereof, including, but not limited to, Fv, Fab, Fab′ and F(ab′) 2 fragments can be used. Such fragments can be produced by enzymatic cleavage or by recombinant techniques. For example, papain or pepsin cleavage can generate Fab or F(ab′) 2 fragments, respectively. Other proteases with the requisite substrate specificity can also be used to generate Fab or F(ab′) 2 fragments. Antibodies can also be produced in a variety of truncated forms using antibody genes in which one or more stop codons have been introduced upstream of the natural stop site. For example, a chimeric gene encoding a F(ab′) 2 heavy chain portion can be designed to include DNA sequences encoding the CH, domain and hinge region of the heavy chain. In some embodiments, agents that specifically bind to a marker protein other than antibodies are used, such as peptides. Peptides that specifically bind to a marker protein can be identified by any means known in the art. For example, specific peptide binders of a marker protein can be screened for using peptide phage display libraries.

In some embodiments, embryo cells can be engineered to over-express one or more genes of interest such as one or more genes differentially abundant in a blastomere (e.g., one or more beta proteins). In some embodiments, the method can comprise providing an expression construct comprising a nucleic acid encoding a gene of interest (e.g., Nedd8 and/or Gps1), and introducing the expression construct into one or more blastomere in a manner permitting expression of the introduced construct in the one or more blastomere, thereby generating at least one engineered blastomere. The expression control element can comprise a promoter, an enhancer, a 5′ un-translated region, a 3′ un-translated region, or any combination thereof. The promoter can be a ubiquitous promoter. The promoter can be a constitutive or an inducible promoter.

“Genetic construct” or “construct” as used herein shall be given their ordinary meanings and can also refer to nucleic acids that comprise a nucleotide sequence which encodes a gene product (e.g., an RNA and/or a protein). The nucleic acid can comprise at least one regulatory element for expression (e.g., an expression control element). The nucleic acid can comprise a vector, such as a viral vector. In some embodiments, the vector can comprise an adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into mammalian cells are available. The vectors may be episomal, e.g. plasmids, virus-derived vectors such cytomegalovirus, adenovirus, etc., or may be integrated into the cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

In addition to viral vectors, a variety of additional tools have been developed that can be used for the incorporation of exogenous genes into cells. One such method that can be used for incorporating polynucleotides encoding target genes into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In certain cases, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems include the piggybac transposon (described in detail in, e.g., WO 2010/085699) and the sleeping beauty transposon (described in detail in, e.g., US2005/0112764), the disclosures of each of which are incorporated herein by reference.

As used herein, the term “expression vector” or “construct” refers to a vector that directs expression of an RNA or polypeptide (e.g., E-cadherin) from nucleic acid sequences contained therein linked to transcriptional regulatory sequences on the vector. The sequences can be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Gene products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1,binding of the EBNA1 protein to the virus replicon region oriP maintains a relatively long-term episomal presence of plasmids in mammalian cells. This particular feature of the oriP/EBNA1vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiment, the vectors can include a regulatory sequence that allows, for example, the translation of multiple proteins from a single mRNA. Non-limiting examples of such regulatory sequences include internal ribosome entry site (IRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is a ration and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited, to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

A promoter may promote ubiquitous expression or tissue-specific expression of an operably linked nucleic acid (e.g., engineered nucleic acid) sequence from any species, including humans. In some embodiments, the promoter is a eukaryotic promoter. Non-limiting examples of eukaryotic promoters include TDH3, PGK1, PKC1, TDH2, PYK1, TPI1,AT1, CMV, EF1 alpha, SV40, PGK1 (human or mouse), Ubc, human beta actin, CAG, TRE, UAS, Ac5, Polyhedrin, CaMKIIa, GAL1, GAL10, TEF1, GDS, ADH1, CaMV35S, Ubi, H1, and U6, as would be known to one of ordinary skill in the art (see, e.g., Addgene website: blog.addgene.org/plasmids-101-the-promoter-region).

Non-limiting examples of ubiquitous promoters include tetracycline-responsive promoters (under the relevant conditions), CMV (EF1 alpha, a SV40 promoter, PGK1, Ubc, CAG, human beta actin gene promoter, a RSV promoter, an EFS promoter, and a promoter comprising an upstream activating sequence (UAS). In certain embodiments, the promoter is a mammalian promoter.

In some embodiments, a promoter of the present disclosure is suitable for use in AAV vectors. See, e.g., U.S. Patent Application Publication No. 2018/0155789, which is hereby incorporated by reference in its entirety for this purpose.

Non-limiting examples of constitutive promoters include CP1, CMV, EF1 alpha, SV40, PGK1, Ubc, human beta actin, beta tubulin, CAG, Ac5,Rosa26 promoter, COL1A1 promoter, polyhedrin, TEF1, GDS, CaM3 5S, Ubi, H1, U6, red opsin promoter (red promoter), rhodopsin promoter (rho promoter), cone arrestin promoter (car promoter), rhodopsin kinase promoter (rk promoter). In some instances, the constitutive promoter is a Rosa26 promoter. In some instances, the constitutive promoter is a COL1A1 promoter.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid (e.g., engineered nucleic acid) in such a way as to be active in inducing transcriptional activity from the inducible promoter. In certain embodiments, an inducing agent is a tetracycline-sensitive protein (e.g., tTA or rtTA, TetR family regulators). Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, e.g., SEQ ID NO: 26, or TetRKRAB, e.g., SEQ ID NO: 27), a tetracycline operator sequence (tetO) and a tetracycline transactivator fusion protein (tTA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al., Nat. Methods. 2012 Feb. 12; 9(3):266-9. Additional non-limiting examples of inducible promoters include mifepristone-responsive promoters (e.g., GAL4-E1b promoter) and coumermycin-responsive promoters. See, e.g., Zhao et al., Hum Gene Ther. 2003 Nov. 20; 14(17):1619-29.

A “reverse tetracycline transactivator” (“rtTA”), as used herein, is an inducing agent that binds to a TRE promoter (e.g., a TRE3G, a TRE2 promoter, or a P tight promoter) in the presence of tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al., Science. 1995 Jun. 23; 268(5218):1766-9 and any of the transactivation domains listed herein). The mutant TetR domain is capable of binding to a TRE promoter when bound to tetracycline. See, e.g., U.S. Provisional Application No. 62/738,894, entitled MUTANT REVERSE TETRACYCLINE TRANSACTIVATORS FOR EXPRESSION OF GENES, which was filed on Sep. 28, 2018, under attorney docket number H0824.70300US00, and is herein incorporated by reference in its entirety.

In some embodiments, embryo cells can be engineered to knock down one or more target genes such as one or more genes differentially abundant in a blastomere (e.g., one or more genes encoding alpha or beta proteins). Various gene knockdown methods can be employed to terminate or reduce the expression of a specific gene, including, for example, RNA interference (RNAi), antisense oligonucleotides, and CRISPR technology. In some embodiments, the method comprises introducing into a blastomere a nucleic acid targeting a gene of interest. The nucleic acid molecule can be single-stranded or double-stranded (e.g., a sense strand and an antisense strand), and comprise a sequence portion corresponding (identical or complementary) to the target gene.

Physical methods for introducing a polynucleotide into a cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). In some embodiments, the nucleic acids described herein are injected into an embryo.

Chemical means for introducing a polynucleotide into a cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a cell. In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution.

Nucleic acids described herein can be introduced into cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

In some aspects, non-viral methods can be used to deliver a nucleic acid described herein into a cell. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition. Exemplary methods of nucleic acid delivery using a transposon include a Sleeping Beauty transposon system (SBTS) and a piggyBac (PB) transposon system. In some embodiments, an engineered described herein are generated by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

The method can also comprise recording a plurality of images of the embryos or sister blastomere of the embryos. The plurality of images may be recorded over a pre-determined period of time, thus illustrating the development from e.g., zygotes to blastocyst embryos. The imaging apparatus may comprise microscopy apparatus, suitable recording apparatus, and optionally image processing apparatus.

Typically, fluorescent markers, such as fluorescent dyes or fluorescent marker proteins, are used in the imaging of embryonic development. Such markers may be added to the culture system. For example, fluorescent dyes may be added to visualize particular molecules or cellular structures. For example, DAPI may be used to stain DNA or MitoTracker (Invitrogen) may be used to stain the mitochondria. Additionally or alternatively, synthetic embryo may produce such fluorescent markers endogenously, e.g., it may contain one or more cells which express a fluorescent marker protein. Such cells may have been genetically modified in order to confer the ability to express such a marker protein. Thus, fluorescence imaging apparatus may be particularly suitable for the methods described. The imaging apparatus may thus comprise a fluorescence microscope, such as a confocal microscope, that can include but is not limited to wide field, scanning and spinning disc confocal, and light sheet microscope.

Confocal microscopes image a single point of a specimen at any given time but allow generation of two dimensional or three dimensional images by scanning different points in a specimen in a regular raster to provide image data which can be assembled into a two or three dimensional image. For example, scanning a specimen in a single plane enables generation of a two dimensional image of a slice through the specimen. A plurality or “stack” of such two dimensional images can be combined to yield a three dimensional image. Spinning disc confocal microscopy provides added advantages over confocal laser scanning microscopy. Additionally, light sheet microscopy can also provide good imaging of embryonic development.

### Applications

The methods and related embryo cells of this disclosure can be applied in multiple ways. Some exemplary applications are described further below.

In some embodiments, the methods and embryos disclosed herein can have a variety of applications including, e.g., investigating mechanisms of embryonic development and for use in treating a subject for a disease or disorder.

In some embodiments, the methods and embryos disclosed herein can be used to identify impact or effect of perturbations or stimuli or mutations on embryonic development. Disclosed herein include methods for detecting perturbation-induced changes in embryo cells. In some embodiments, the method can comprise introducing a perturbation to an embryo at the zygote stage, determining a protein composition of the embryo according to the methods described herein, and detecting the perturbation-induced change on the protein composition of single blastomeres. In some embodiments, the detection comprises comparing the protein composition obtained in the presence of the perturbation with a protein composition obtained in the absence of the perturbation. In some embodiments, the detection comprises comparing the identities and/or abundances of the differentially abundant proteins identified between the sister blastomeres derived from a same embryo. In some embodiments, the differentially abundant proteins comprises one or more alpha and/or beta proteins.

The perturbation can be a chemical perturbation or a physical perturbation. The perturbations may comprise one or more genetic perturbation. For example, the perturbation can comprise gene knock-down, gene knock-out, gene activation, gene insertion, and/or regulatory element deletion. In some embodiments, the perturbation can comprise genome-wide perturbation. The perturbation may comprise one or more epigenetic or epigenomic perturbation. The perturbation can be a single-order perturbation. Alternatively, the perturbation can comprise combinatorial perturbations. The perturbations can be of a selected group of targets based on similar pathways or network of targets. In some embodiments, the perturbation can target one or more proteins identified to be differentially abundant in the sister blastomeres of an embryo.

In some embodiments, the perturbation can be introduced via a test agent such as a chemical agent or biological agent. Examples of test agents include, but are not limited to, an antibiotic, a small molecule, a growth factor, a hormone or a derivative thereof, a steroid or a derivative thereof, infectious agents (e.g., infections, viruses, parasites and other bacterial illnesses), and toxic chemicals such as organic mercury, lead, pesticides, and herbicides. In some embodiments, the test agent is a teratogen. A teratogen is a substance that interferes with normal fetal development and causes congenital disabilities. Examples of teratogens include, but are not limited to, drugs (e.g., antiepileptic drugs, antimicrobials, anticoagulants, antithyroid medications, vitamin A, or hormonal medication), alcohol, recreational drugs, chemicals and toxic substances. In some embodiments, the perturbation is an exposure of an embryo to a drug candidate. Drug candidates can comprise small molecules, hormones, peptides, proteins, nucleic acids, or any combination thereof. Therefore, in some embodiments, the methods disclosed herein can be used for screening perturbations, including compounds or agents or conditions, capable of modifying a proteomic profile or protein composition of an embryo or a sister blastomere of the embryo. The method can comprise performing the method as described herein, wherein the perturbation comprises exposing the embryo or the sister blastomere of the embryo to each compound or agent or condition.

In some embodiments, the perturbation can be an increase or decrease of temperature across a gradient, addition or subtraction of energy, electromagnetic energy, or ultrasound. Other perturbations include exposure to light, movement, agitation, exposure to cellular material derived from other tissues, organisms, or microorganisms, or diseased cell lines.

In some embodiments, the perturbation can be introduced with RNA interference or a CRISPR-Cas system to a sister blastomere. In some embodiments, the perturbation can comprise performing single or combinatorial CRISPR-Cas-based perturbation with a genome-wide library of guide RNAs. For example, each guide RNA can be associated with a unique perturbation barcode. Each gRNA may be co-delivered with a reporter mRNA comprising the unique perturbation barcode. The method may comprise an array-format or pool-format perturbation. In some embodiments, introducing a perturbation to a blastomere comprises contacting the perturbation (e.g., a test agent or a drug candidate) with a sister blastomere or an embryo in a culture media or culturing a sister blastomere or an embryo in a culture media in the presence of the perturbation or under a perturbed condition such as under an elevated temperature, pressure, pH value or other modified physiological conditions. The term “physiological conditions,” as used herein, can refer to a range of chemical (e.g., pH, ionic strength), biochemical (e.g., enzyme concentrations), and physical (e.g., temperature, pressure) conditions that can be encountered in intracellular and extracellular fluids of tissues, such as, for example, in the intracellular and extracellular fluids of a subject. For most cells and tissues, the physiological pH ranges from about 7.0 to about 7.5, the physiological ionic strength ranges from about 50 mM to about 400 mM, the physiological temperature ranges from about 20° C. to about 42° C., and the physiological pressure ranges from about 925 mbar to about 1050 mbar.

In some embodiments, a perturbation can induce an adverse effect on the embryonic development by increasing or decreasing one or more proteins differentially abundant among sister blastomeres. For example, the perturbation can induce an adverse effect on the embryonic development by decreasing the abundance of one or more beta proteins and/or increasing the abundance of one or more alpha proteins in a test blastomere by about, at least, at least about 10%, 15%, 20%, 25%, 30%, 35%, 40%, 45%, 50%, 55%, 60%, 65%, 70%, 75%, 80%, 85%, 90%, 95%, or greater, compared to that in a control blastomere in the absence of the perturbation. In some embodiments, the perturbation is considered harmful or toxic to the embryonic development if the perturbation decreases the abundance of one or more beta proteins in a test blastomere by about, at least, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the perturbation is considered harmful or toxic to the embryonic development if the perturbation increases the abundance of one or more alpha proteins in a test blastomere by about, at least, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values).

Provided herein also include differentiated cells obtainable by any of the methods disclosed herein. The method can comprise: removing one or more blastomeres from the embryo at various developmental stages (e.g., two-cell stage, four-cell stage, eight-cell stage, and so on); and culturing the one or more blastomeres to produce differentiated cells. In some embodiments, the differentiated cells are selected from the group comprising exocrine secretory epithelial cells, hormone secreting cells, cells of the integumentary system, cells of the nervous system, metabolism and storage cells, barrier function cells, extracellular matrix cells, contractile cells, blood and immune system cells, germ cells, nurse cells and interstitial cells.

Examples of differentiated cells include cells that are derived primarily from the endoderm, cells that are derived primarily from the ectoderm and cells that are derived primarily from the mesoderm and cells that are derived primarily from the germ line. Cells that are derived primarily from endoderm include exocrine secretory epithelial cells and hormone secretory cells. Exocrine secretory epithelial cells include salivary gland cell, von Ebner's gland cell in tongue, mammary gland cell, lacrimal gland cell, ceruminous gland cell in ear, eccrine sweat gland dark cell, eccrine sweat gland clear cell, apocrine sweat gland cell, gland of Moll cell in eyelid, sebaceous gland cell, Bowman's gland cell in nose, Brunner's gland cell in duodenum, seminal vesicle cell, prostate gland cell, bulbourethral gland cell, Bartholin's gland cell, gland of Littre cell, uterus endometrium cell, isolated goblet cell of respiratory and digestive tracts, stomach lining mucous cell, gastric gland zymogenic cell, gastric gland oxyntic cell, pancreatic acinar cell, paneth cell of small intestine, type II pneumocyte of lung and Clara cell of lung. Hormone secreting cells include anterior pituitary cells, intermediate pituitary cell, magnocellular neurosecretory cells, gut and respiratory tract cells, thyroid gland cells, parathyroid gland cells, adrenal gland cells, Leydig cell of testes, Theca interna cell of ovarian follicle, corpus luteum cell, juxtaglomerular cell, macula densa cell of kidney, peripolar cell of kidney and Mesangial cell of kidney.

Cells that are derived primarily from ectoderm include cells of the integumentary system and nervous system. Cells of the integumentary system include keratinizing epithelial cells (such as epidermal keratinocyte, epidermal basal cell, keratinocyte of fingernails and toenails, nail bed basal cell, medullary hair shaft cell, cortical hair shaft cell, cuticular hair shaft cell, cuticular hair root sheath cell, hair root sheath cell of Huxley's layer, hair root sheath cell of Henle's layer, external hair root sheath cell, hair matrix cell), wet stratified barrier epithelial cells (such as surface epithelial cell of stratified squamous epithelium of cornea, tongue, oral cavity, esophagus, anal canal, distal urethra and vagina, basal cell of epithelia of cornea, tongue, oral cavity, esophagus, anal canal, distal urethra and vagina and urinary epithelium cell). Cells of the nervous system include sensory transducer cells (such as auditory inner hair cell of organ of Corti, auditory outer hair cell of organ of Corti, basal cell of olfactory epithelium, cold-sensitive primary sensory neurons, heat-sensitive primary sensory neurons, Merkel cell of epidermis, olfactory receptor neuron, pain-sensitive primary sensory neurons, photoreceptor cells of retina in eye, proprioceptive primary sensory neurons, touch-sensitive primary sensory neurons, type I carotid body cell, type II carotid body cell, type I hair cell of vestibular system of ear, type II hair cell of vestibular system of ear and type I taste bud cell), autonomic neuron cells (such as cholinergic neural cell, adrenergic neural cell and peptidergic neural cell), sense organ and peripheral neuron supporting cells (such as inner pillar cell of organ of Corti, outer pillar cell of organ of Corti, inner phalangeal cell of organ of Corti, outer phalangeal cell of organ of Corti, border cell of organ of Corti, Hensen cell of organ of Corti, vestibular apparatus supporting cell, taste bud supporting cell, 5 olfactory epithelium supporting cell, Schwann cell, satellite glial cell and enteric glial cell), central nervous system neurons and glial cells (such as astrocyte, neuron cells, oligodendrocyte and spindle neuron) and lens cells (such as anterior lens epithelial cell and crystallin-containing lens fiber cell).

Cells that are derived primarily from mesoderm include metabolism and storage cells, barrier function cells, extracellular matrix cells, contractile cells, blood and immune system cells, germ cells, nurse cells and interstitial cells. Metabolism and storage cells include hepatocyte, adipocytes and liver lipocyte. Barrier function cells (lung, gut, exocrine glands and urogenital tract) include kidney cells (such as kidney parietal cell, kidney glomerulus podocyte, kidney proximal tubule brush border cell, loop of Henle thin segment cell, kidney distal tubule cell, kidney collecting duct cell, type I pneumocyte, pancreatic duct cell, nonstriated duct cell, duct cell, intestinal brush border cell, exocrine gland striated duct cell, gall bladder epithelial cell, ductulus efferens nonciliated cell, epididymal principal cell and epididymal basal cell). Extracellular matrix cells include ameloblast epithelial cell, planum semilunatum epithelial cell of vestibular system of ear, organ of Corti interdental epithelial cell, loose connective tissue fibroblasts, corneal fibroblasts, tendon fibroblasts, bone marrow reticular tissue fibroblasts, other nonepithelial fibroblasts, pericyte, nucleus pulposus cell of intervertebral disc, cementoblast/cementocyte, Odontoblast/odontocyte, hyaline cartilage chondrocyte, fibrocartilage chondrocyte, elastic cartilage chondrocyte, osteoblast/osteocyte, osteoprogenitor cell, hyalocyte of vitreous body of eye, stellate cell of perilymphatic space of ear, hepatic stellate cell and pancreatic stelle cell. Contractile cells include skeletal muscle cells including red skeletal muscle cell, white skeletal muscle cell, intermediate skeletal muscle cell, nuclear bag cell of muscle spindle and nuclear chain cell of muscle spindle, satellite cells, heart muscle cells including ordinary heart muscle cell, nodal heart muscle cell and Purkinje fiber cell, smooth muscle cell, myoepithelial cell of iris and myoepithelial cell of exocrine glands. Blood and immune system cells include erythrocyte, megakaryocyte, monocyte, connective tissue macrophage, Langerhans cell, osteoclast, dendritic cell, microglial cell, neutrophil granulocyte, eosinophil granulocyte, basophil granulocyte, hybridoma cell, mast cell, helper T cell, suppressor T cell, cytotoxic T cell, natural Killer T cell, B cell, natural killer cell, reticulocyte and committed progenitors for the blood and immune system. Germ cells include oogonium/Oocyte, spermatid, spermatocyte, spermatogonium cell and spermatozoon. Nurse cells include ovarian follicle cell, sertoli cell and thymus epithelial cell, and interstitial cells include interstitial kidney cells.

The disclosed compositions and methods may be used to produce differentiated cells from any suitable mammalian species, such as: primates, including humans, great apes (e.g., gorillas, chimpanzees, orangutans), old world monkeys, new world monkeys; rodents (e.g., mice, rats, guinea pigs, hamsters); cats; dogs; lagomorphs (including rabbits); cows; sheep; goats; horses; pigs; and any other livestock, agricultural, laboratory or domestic mammals. The presently disclosed compositions and methods may be used to produce differentiated cells from any non-human mammal, including but not limited to those described above.

The embryos and differentiated cells disclosed herein can have a variety of applications including, e.g., investigating mechanisms of embryonic development and for use in treating a subject for a disease or disorder.

Disclosed herein include methods of identifying a compound useful for treating a disease. In some embodiments, the method comprises contacting an embryo, a blastomere, or a differentiated cell(s) obtainable by any of the methods provided herein with the compound.

Disclosed herein include methods for diagnosing or treating a disease or disorder in a subject. In some embodiments, the method comprises use of an embryo, a blastomere, or a differentiated cell(s) obtainable by the methods provided herein, or any combination thereof. Also provided are methods of elucidating the role of a gene in embryo development. In some embodiments, the method comprises obtaining an embryo, a blastomere or a differentiated cell(s) where the gene has been modified or knocked out, such as one of the genes differentially abundant in sister blastomeres, and culturing the cell to obtain a plurality of cells for use.

### EXAMPLES

Some aspects of the embodiments discussed above are disclosed in further detail in the following examples, which are not in any way intended to limit the scope of the present disclosure.

### Example 1

### General Experimental Methods

This example describes general methods, culturing conditions, and related mass-spectrometry and proteomics analysis used in Examples 2-8 below.

### Mouse Embryo Culture and Sample Collection

These experiments adhered to the regulations of the Animals (Scientific Procedures) Act 1986—Amendment Regulations 2012 and was reviewed by the University of Cambridge Animal Welfare and Ethical Review Body. Experiments were approved by the UK Home Office.

Embryos were collected from 4-6 week old F1 females (C57BI6 x CBA, Charles River) following superovulation by injection of 5 IU of pregnant mares' serum gonadotropin (PMSG, Intervet) and 5 IU of human chorionic gonadotropin (hCG, Intervet) 48 hrs later. Females were mated with F1 males (6 weeks-52 weeks of age, C57BI6 x CBA, Charles River). Plugged females were culled by cervical dislocation to recover embryos at the required stage. Embryos, other than zygotes, were recovered in M2 medium (in house).

Zygote stage: zygote stage embryos (22 hrs post-hCG) were recovered in M2 medium with 1 mg/ml of hyaluronidase (Sigma, H4272) in order to remove cumulus cells and subsequently washed through M2 medium without hyaluronidase. Samples were collected at 23-24 hrs post-hCG.

Early 2-cell stage: zygote stage embryos (29 hrs post-hCG) were recovered in hyaluronidase as above and subsequently cultured for 1-3 h during division from the zygote to 2-cell stage. Following division to the 2-cell stage, samples were collected at 30-32 hrs post-hCG.

Late 2-cell stage: 2-cell stage embryos were recovered at 45 hrs post-hCG and samples collected at 46-48 hrs post-hCG.

4-cell stage: mid to late 2-cell stage embryos were recovered and one sister blastomere was microinjected as described below, to allow for identification of the 4-cell stage sisters originating from the injected 2-cell stage blastomere (mCherry positive pairs were distinguished by SCoPE2). The embryos were transferred to KSOM (Merck, MR-106-D), and live imaged during division from the 2- to 4-cell stage and collected at 55-57 hrs post-hCG. Division pattern, age post-division (2- to 4-cell stage) and division order were annotated for each embryo prior to collection. Uninjected and unimaged controls were also collected with the same timings.

The zona pellucida was removed prior to blastomere separation by brief acidic Tyrode's solution treatment (Sigma, T1788), followed by washes in M2 media. Embryos were then transferred to 35 mm petri dishes (Corning, 351008) coated in 1% agarose and covered with M2 media. Blastomeres were separated from each other using a thin glass capillary and transferred immediately to M2 medium. Separation took up to 1 min and had a survival rate greater than 80%. Zygotes were split in a similar manner to give rise to two intact split halves. Zygotes were split meridionally in alignment with the animal-vegetal axis as defined by the position of the polar body. After the embryos had been split, individual blastomeres or zygote halves were washed through 7-10 washes of PBS (Life Technologies, 10010056) followed by 5 washes in pure water (Optima LC/MS Grade, Fisher Scientific, W6500), before being finally resuspended in 1 μl of water and transferred to individual wells of a 384 well plate (ThermoFisher, AB1384), on a cold block. In order to minimize sample contamination all surfaces were cleaned and filter tips used. Wash drops were not reused more than 8 times and were changed if a blastomere lysed. Two different glass pipettes were used for the PBS and water washes to prevent carry over. In each experiment, sample collection took up to 1 hr and plates were subsequently sealed with foil (ThermoFisher, AB0626).

For PCNA live imaging experiments, zygotes were recovered as described above and microinjected. Following microinjection, embryos were cultured in KSOM media (Merck, MR-106-D) under mineral oil (Biocare Europe) at 5% CO₂, and 37° C. overnight and allowed to cleave to the 2-cell stage. Prior to imaging, 2-cell embryos were split as above and transferred to an imaging dish. In experiments where further culture was required following imaging, split embryos were transferred to Global Total culture medium (LifeGlobal group, H5GT-030) under mineral oil (Biocare Europe) at 5% CO2, and 37° C., for 72 hrs.

For split embryo culture to the blastocyst stage, 2-cell stage embryos were split as above and single blastomeres cultured in drops of Global Total culture medium (LifeGlobal group, H5GT-030) under mineral oil (Biocare Europe) at 5% CO₂, and 37° C., for 72 hrs.

For knockdown and overexpression experiments, zygote or 2-cell stage embryos were recovered as described above and microinjection performed. Following microinjection embryos were cultured in KSOM media under mineral oil at 5% CO₂, and 37° C., until the required stage.

dsRNA and mRNA Synthesis

The mCherry sequence was amplified via PCR from the pRN3P-H2B-mCherry vector and cloned into the pRN3p vector via EcoRI/BamHI digestion (ThermoFisher, FD0054and FD0274) and T4 ligation (New England Biolabs, M0202S). pRN3P-mCherry was linearized using KpnI (New England Biolabs, R3142S). In vitro transcription was carried out using the mMessage mMachine T3 kit (Thermo Fisher, AM1348) and purified via lithium chloride precipitation, according to the manufacturer's instructions. pRN3P-Gap43-RFP and pRN3P-PCNA-Clover were linearized using SfiI (ThermoFisher, FD1824) and mRNA synthesized via in vitro transcription using T3 as above.

dsRNAs of 350-500 bp length, were designed using the E-RNAi platform (Horn and Boutros, 2010) and amplified from mouse liver cDNA. In vitro transcription was carried out using the MEGAscript T7 kit (Thermo Fisher, AM1334) and purified via lithium chloride precipitation, according to the manufacturer's instructions.

For overexpression experiments, Nedd8 and Gps1 sequences were amplified via PCR from mouse liver cDNA, and restriction sites and an HA tag added via PCR. Sequences were cloned in the pRN3p vector via EcoRI/BamHI (ThermoFisher, FD0274 and FD0054) and HindIII/BamHI (ThermoFisher, FD0505 and FD0054) digestion for Nedd8-HA and Gps1-HA respectively, followed by T4 ligation (New England Biolabs, M0202S). pRN3P-Nedd8-HA and pRN3P-Gps1-HA were linearised using SdaI (ThermoFisher, FD1194).

In vitro transcription using T3 was carried out as above. Primers used for dsRNA and mRNA synthesis are listed in Table 3 below.

TABLE 3
Exemplary primers for dsRNA and mRNA synthesis

| | | SEQ |
|---|---|---|
| | | ID |
| Primer | Sequence (5′ to 3′) | NO |
| mCherry EcoRI F | ACGTGAATTCATGGTGAGCAAGGGCGAG | 1 |
| | G | |
| mCherry BamHI | RACGTGGATCCCTACTTGTACAGCTCGT | 2 |
| | CCATGCC | |
| dsGps1 F | ACGTTAATACGACTCACTATAGGGATTC | 3 |
| | TATGAATCCAAGTATGCCTCA | |
| dsGps1 R | ACGTTAATACGACTCACTATAGGGACAG | 4 |
| | CTGCTCTCAGAATCATAGC | |
| dsNedd8 F | ACGTTAATACGACTCACTATAGGGGGG | 5 |
| | AGAAGCAGCACTCTAGC | |
| dsNedd8 R | ACGITAATACGACTCACTATACGGTC | 6 |
| | TGGTGTCCCAGAGAGTGA | |
| dsPSMC4 F | ACGTAATACGACTCACTATAGGGGCC | 7 |
| | CAGGAGGAGGTGAAG | |
| dsPSMC4 R | ACGTAATACGACTCACTATAGGGATC | 8 |
| | GATGCCAATCTGCTTGT | |
| Gapdh qPCR F | CGTATTGGGCGCCTGGTCAC | 9 |
| Gapdh qPCR R | ATGATGACCCTTTTGGCTCC | 10 |
| Gps1 qPCR F | GATCCATGTCAAGTCTCCTCCT | 11 |
| Gps1 qPCR R | CTGTTGGCTGGAGTCAGCTC | 12 |
| Nedd8 qPCR F | TACTGGTGGGAGAATGTGAGG | 13 |
| Nedd8 qPCR R | TAAGACAGGGAAGCACACATGA | 14 |
| PSMC4 qPCR F | CAGCACTGTCCGTGTCTCG | 15 |
| PSMC4 qPCR R | CTGCTCGTCCTTGATATACTCCTC | 16 |
| Gps1 F | CGCGTCAGGCCAACAT | 17 |
| Gps1 HindIII F | ACGTAAGCTTCCGCGTCAGGCCAACAT | 18 |
| Gps1-HAR | TCATACCCATACGATGTTCCAGATTAC | 19 |
| | GCTCATGTTGGTACTCATGCG | |
| Nedd8 EcoRI F | ACGTGAATTCATGCTAATTAAAGTGAA | 20 |
| | GACGCTGACTG | |
| Nedd8-HA R | TACCCATACGATGTTCCAGATTACGCT | 21 |
| | CTGCCCAAGAC | |
| HA BamHI R | ACGTGGATCCTCATACCCATACGATGT | 22 |
| | TCCAGATTACGCT | |

### Microinjection

Microinjection was performed as previously described (Zernicka-Goetz, M et al., Development 124, 1133-1137 (1997)). Briefly, embryos were placed in a depression on a glass slide in M2 medium covered with mineral oil (Biocare Europe, 9305). Microinjection was performed using an Eppendorf Femtojet Microinjector with negative capacitance to facilitate membrane entry. Synthetic mCherry mRNA and Gap43-RFP were injected at a concentration of 200 ng/μl. For PCNA live imaging experiments, mRNA was injected at 100 ng/μl. For knockdown experiments dsRNAs were injected at a concentration of 1000 ng/μl. For overexpression, mRNA was injected at the indicated concentration (50 ng/μl or 500 ng/μl).

qRT-PCR

RNA was collected from embryos at 48 hrs post-injection, using the Arcturus PicoPure RNA isolation kit (Arcturus Bioscience, KIT0204), according to the manufacturer's instructions. Quantitative reverse transcriptase polymerase chain reaction (qRT-PCR) was performed using a StepOne Plus Real-time PCR machine (Applied Biosystem) and the Power SYBR Green RNA-to-CT 1-Step Kit (Life Technologies, 4389986). Relative mRNA expression levels of genes of interest were calculated using the ddCT method, with normalization to Gapdh.

### Immunofluorescence and Confocal Imaging

Blastocyst or 8-cell stage embryos were fixed in 4% PFA for 20 min at room temperature, and then washed through PBST (0.1% Tween 20 (Sigma Aldrich) in PBS) three times. Embryos were then permeabilized in 0.5% Triton X-100 (Sigma Aldrich) in PBS for 20 min at room temperature and washed through PBST again, before being transferred to blocking buffer (3% bovine serum albumin (SIgma Aldrich) in PBST) for 3 hrs at 4° C. Samples were then incubated with primary antibody mixes (diluted in blocking buffer) overnight at 4° C. The next day, embryos were washed through PBST and incubated in secondary antibody mixes (1:500 in blocking buffer) with DAPI (Life Technologies, D3571, 1:1000 dilution, in PBST) for 2 hrs at room temperature. Finally, samples were washed through PBST following incubation with secondary antibodies and then imaged. Imaging was carried out on a SP5 or SP8 scanning confocal microscope (Leica) using the 63× or 40× oil objective.

Primary antibodies used: goat monoclonal anti Sox17 (R&D Systems, af1924,1:200), mouse monoclonal anti Cdx2 (Launch Diagnostics, MU392-UC (Biogenex), 1:200), rat anti HA (Roche, 11867423001, 1:100), rabbit anti-Nanog (Abcam ab80892, 1:200) and rabbit anti RFP (Rockland, 600-401-379, 1:500)

Secondary antibodies used: Alexa Fluor 488 Donkey anti-Mouse, (ThermoFisher Scientific, A21202); Alexa Fluor 568 Donkey anti-Rabbit (ThermoFisher Scientific, A10042) and Alexa Fluor 647 Donkey anti-Goat (ThermoFisher Scientific, A21447). All secondaries were used at a 1:400 dilution.

### Image Analysis and Statistics

Images were processed with Fiji software (2012, imagej.net/software/fiji/) to assess cell number and lineage allocation. Cell numbers were counted manually, using the multi-point counter function. For HA tag staining, regions of interest were defined for the nucleus and cytoplasm at the midplane of each cell and intensity was measured using the ImageJ measure function. HA tag intensity was normalised to DAPI intensity.

The statistical test used is indicated in the corresponding figure legend. In all cases, the two-tailed version of the test was used. Normality of the data was assessed using the Shapiro Wilk test. Statistical analysis was performed using Prism software (version 8, GraphPad, https://www.graphpad.com/scientific-software/prism/).

### Live Imaging

4-cell stage classification: Live imaging was performed with a SP5 scanning confocal microscope (Leica) using the 63× oil objective. 2- to 4-cell mouse embryos were imaged on glass-bottom dishes (MatTek, P35G-1.5-14-C) within a nylon mesh (Plastok) in KSOM media under mineral oil and kept in a humidified chamber at 5% CO₂, and 37° C. throughout imaging. Images were captured every 15 mins with a z-step size of 5 μm. Time lapse recordings were processed with Fiji software to assess division order, division timing and division pattern.

PCNA cell cycle assessment: Imaging was performed with a spinning disk confocal microscope (3i Intelligent Imaging Innovations) using the 63× oil objective, from 36 to 46 hrs post-hCG during the transition from S to G2 phase. Single blastomeres from split 2-cell embryos were imaged on glass bottom dishes, within a nylon mesh in KSOM media under mineral oil, at 5% CO₂, and 37° C. The imaging interval was 15 mins and the z-step size 5 μm. After imaging a total of 30 single 2-cell stage blastomeres (15 split embryos) were collected for subsequent MS analysis as above. In a separate set of experiments blastomeres were allowed to undergo the second cleavage division during imaging and then removed from the imaging chamber and cultured to the blastocyst stage. Images were exported from SlideBook (3i Intelligent Imaging Innovations) and subsequently processed with Fiji software to assess S phase exit and division order when possible.

### Mouse Stem Cell Culture and Sample Collection

CAG-GFP/tetO-H2B-mCherry mouse embryonic stem cells (ESCs) were used as carrier samples for the SCoPE2. Cells were cultured on gelatin coated plates at 5% CO₂, and 37° C. in N2B27 2iLIF media. N2B27 2iLIF was comprised of 50% Neurobasal-A (Gibco, 10888022), 50% DMEM/F-12 (Gibco, 21331020), 0.5% N2 (in-house), 1% B27 (Gibco, 10889038), 2 mM GlutaMAX (Gibco, 35050038), 0.1 mM 2-mercaptoethanol (Gibco, 31350010) and 1% penicillin/streptomycin (Gibco, 15140122), with 3 mM CHIR99021 (Cambridge Stem Cell Institute), 1 mM PD0325901 (Cambridge Stem Cell Institute) and 10 ng ml-1 leukaemia inhibitory factor (Cambridge Stem Cell Institute) supplemented.

To induce H2B-mCherry expression, CAG-GFP/tetO-H2B-mCherry ESCs were treated with Doxycycline (1 mg/mL) (Sigma-Aldrich, D9891-5G) for 6 hrs prior to collection.

ESCs were routinely passaged at 70% confluency following trypsinisation (Trypsin-EDTA 0.05%, Life Technologies, 25300054) for 4 minutes at 37° C. Feeder cell media was added to terminate the trypsinization and cells were dissociated by gentle pipetting and centrifuged for 4 minutes at 1000 rpm, before being re-plated at a 1:10 or 1:20 dilution. Feeder cell medium contained Dulbecco's modified essential medium (Gibco, 41966052), 15% fetal bovine serum (Cambridge Stem Cell Institute), 1 mM sodium pyruvate, 2 mM GlutaMAX, 1% MEM non-essential amino acids (Gibco, 11140035), 0.1 mM 2-mercaptoethanol and 1% penicillin/streptomycin. Cells were routinely tested for mycoplasma contamination.

For sample collection, cells were trypsinized as above, and resuspended in PBS (Life Technologies, 10010056). The cells were then pelleted by centrifugation for 4 minutes at 1000 rpm before a second PBS wash. A haemocytometer was then used to estimate cell density and cells were pelleted as above, before being finally resuspended in pure water at a density of 2000 cells/μl. 200,000-300,000 cells total were collected in 0.2 ml PCR tubes (Starlab, A1402-3700) and stored at −80° C.

### Human Stem Cell Culture and Sample Collection

The use of human ESCs (hESCs) was approved by the UK Stem Cell Bank Steering Committee and experiments complied with the UK Code of Practice for the Use of Human Stem Cell Lines. RUES2 hESCs (kindly provided by Ali Brivanlou) were used as carrier samples for SCoPEi2. All cells were routinely tested for mycoplasma contamination.

RUES2 hESCs were cultured in a humidified incubator at 37° C. and 5% CO₂ in mTeSR1 (StemCell Technologies, 85850) on growth factor-reduced Matrigel-coated (Corning, 353046). For Matrigel coating, plates were incubated with 0.16 mg/ml Matrigel in DMEM/F12 (Gibco, 21331020) at 37° C. for 1 hour. Media was changed daily.

hESCs were routinely passaged every 4-5 days by dissociating in Accutase (ThermoFisher Scientific, A1110501) for 3 minutes at 37° C. Cells were collected in DMEM/F12and centrifuged for 3 minutes at 1000 rpm before being re-plated in mTesR1 medium supplemented with 10 μM ROCK inhibitor Y-27632 (StemCell Technologies, 72304) for 24 hours.

For sample collection, cells were dissociated as above for routine passage. RUES2 hESCs were resuspended in PBS. Cells were pelleted by centrifugation for 4 minutes at 1200 rpm before a second PBS wash. Cells were resuspended in pure water at a density of 2000 cells/μl. 200,000-300,000 cells total were collected in 0.2 ml PCR tubes and stored at −80° C.

### Ethics Statement

Human embryo samples for this study were collected in two different institutes: the University of Cambridge (United Kingdom), and the California Institute of Technology (United States). All the work complies with The International Society for Stem Cell Research (ISSCR) guidelines.

Human embryo work at the University of Cambridge was performed in accordance with Human Fertility and Embryology Authority (HFEA) regulations (license reference R0193).

Ethical approval for the work was obtained from the ‘Human Biology Research Ethics Committee’ at the University of Cambridge (reference HBREC.2017.21). Informed consent was obtained from all participants in the study which included patients from the CARE Fertility Group and Herts & Essex fertility clinics. Supernumerary embryos were donated upon completion of IVF treatment. Patients were informed about the specific objectives of the project, and the conditions that apply within the license, before giving consent. Patients were also offered counseling and did not receive any financial inducements for their donation.

Human embryo work at the California Institute of Technology was approved by the California Institute of Technology Committee for the Protection of Human Subjects (IRB number 19-0948). Human embryos at the two pronuclei stage were obtained from the University of Southern California (USC) through the pre-existing USC IRB-approved Biospecimen Repository for Reproductive Research (HS-15-00859) after appropriate approval was obtained unanimously from the Biorepository Ethics Committee. Supernumerary embryos were donated upon completion of IVF treated from USC Fertility. Patients were informed of the general conditions of the donation, as well as the objectives, and methodology of human embryo research. They were offered counseling and alternatives to donation, including discarding embryos and continued cryopreservation of embryos. Patients were informed that they would not benefit directly from the donation of embryos to research.

### Human Embryo Culture and Sample Collection

A total of 8 donated two pronuclei stage human zygotes (day 1 post fertilization) from two patients were used for this study. Embryos were warmed and cultured according to the above regulations. Cryopreserved day 1 embryos were thawed with the Origio thaw kit (REF10984010A) following the manufacturer's instructions. Briefly, the Global Total human embryo culture medium (LifeGlobal group, H5GT-030) was incubated at 37° C. and 5% CO₂ overnight before thawing. The next day, the straw containing the embryo was immersed in prewarmed (37° C.) water for 1 min. The embryo was then transferred into vial 1 (5 min), vial 2 (5 min), vial 3 (10 min), and finally into vial 4. Thawed embryos were finally incubated in drops of the pre-equilibrated Global Total human embryo culture medium under mineral oil (Irvine Scientific, 9305). Embryos were incubated for a total of 12 hrs overnight at 37° C., and 5% CO₂. The following day the zona pellucida of the 2-cell stage human embryos was removed by brief acidic Tyrode's solution treatment (Sigma, T1788).

Embryos were then bisected and single blastomeres transferred to M2 media before being washed and processed as above into 384-well PCR plates (ThermoFisher AB1384) in 1 μl of pure water (Optima LC/MS Grade, Fisher Scientific W6500).

Of 8 two pronuclei zygote stage human embryos, 7 embryos developed to the 2-cell stage for sample collection and 5 were included in the final analysis.

A total of 22 donated two pronuclei stage human zygotes (day 1 post fertilization) from 5 patients were used for this study. Cryopreserved day 1 embryos were warmed with the Embryo Thaw Media Kit following the manufacturer's instructions (Fujifilm Irvine Scientific, 90124). Briefly, the Continuous Single Culture-NX Complete medium (Fujifilm Irvine Scientific, 90168) was incubated at 37° C. and 5% CO₂ overnight before thawing. The next day, the straw containing the embryo was defrosted at room temperature for 30 s and immersed in prewarmed (37° C.) water for 1 min until the ice melted. The embryo was then transferred into T-1 (5 min), T-2 (5 min) and T-3 (10 min) solutions for slow thawing, before being finally transferred to Multipurpose Handling Medium (Fujifilm Irvine Scientific, 90163) for recovery. Thawed embryos were then incubated in drops of pre-equilibrated Continuous Single Culture-NX Complete medium under mineral oil (Irvine Scientific, 9305). Embryos were incubated at 37° C., and 5% CO₂ for 6-12 hrs until they reached the 2-cell stage.

Assisted hatching was performed using laser pulses at 200 μs (Lykos laser: Hamilton Thorne, Beverly, MA, USA). Embryo Biopsy Medium (Irvine Scientific, 90103) was then used to separate the blastomeres from each other, followed by gentle pipetting of the embryo to remove it from the zona pellucida and isolate individual sister blastomeres. Blastomeres were then washed and processed as above into 384-well PCR plates (ThermoFisher, AB1384) in 1 μl of pure water (Optima LC/MS Grade, Fisher Scientific, W6500).

Of 22 two pronuclei zygote human embryos, 20 embryos developed to the 2-cell stage for sample collection and 8 were included in the final analysis.

### Single-Cell RNA Sequencing Analysis

Single-cell RNA-sequencing analysis was performed on early, mid, and late 2-cell stage blastomeres using the previously published dataset by Deng et al. (GSE45719). Reads were aligned against the reference genome GRCm39 and count matrices were made using kallisto|bustools. Further downstream analyses were performed in Python using the Scanpy toolkit (version 1.9.1) and Anndata (version 0.8.0). None of the cells were filtered for mitochondrial or ribosomal content. Initial analysis including normalization, scaling, and identification of highly variable genes was performed using the Scanpy preprocessing toolkit. Single-cell data was further visualized using several Scanpy plotting tools including: sc.pl.umap, sc.pl.clustermap, and sc.pl.heatmap.

Mass Spectrometry (MS) Sample Preparation (SCoPE2 and pSCoPE Sample Preparation)

Isobaric carrier & reference. Ample preparation and analysis was performed as described by Petelski et al. Briefly, mouse embryonic stem cells at a density of 2,000 cells/μl in 100 μl water were lysed through the mPOP method (frozen cells were subjected to a rapid heat cycle of 90° C. for 10 minutes, and then were cooled to 12° C.). Trypsin Gold (TG, Thermo) and triethylammonium bicarbonate (TEAB, pH=8, Sigma) were added to the cell lysate to final concentrations of 10 ng/μl and 100 mM, respectively. The sample, once mixed with TG and TEAB, was subjected to 37° C. overnight (16-18 hours) to digest proteins into peptides. To ensure adequate miscleavage rate (<20%), a small amount (1 μl) of the cell digest was evaluated via LC-MS/MS. The cell digest was split into two samples, one for the carrier and the other for the reference. The carrier was labeled with TMT 126 and the reference was labeled with TMT 127N, with the labeling reaction proceeding for 1 hour. The reaction was quenched with 1% hydroxylamine (HA) for 30 minutes. Labeled material was then evaluated via LC-MS/MS for labeling efficiency (>99%). Carrier and reference materials were kept frozen −80° C. until needed for multiplexing with single cells.

Single blastomere cells and half zygotes. Frozen blastomeres that were collected in a 384-well plate were lysed by rapidly heating in a thermocycler to 90° C. for 10 minutes and then cooled to 12° C. To each well (with a single blastomere or a water serving as the control), TG and TEAB were added to the cell lysate to final concentrations of 10 ng/μl and 100 mM, respectively. The plate was then subjected to 37° C. for three hours. Each well then received 0.5 μl of selected TMT reagents and the plate was incubated at room temperature for 1 hour. The labeling reaction was quenched with 0.5 μl of 1% HA at room temperature for 30 minutes. Single blastomeres were then combined with 200 carrier and 5 reference cells to form a TMT set (FIG. 7, panel c). Each TMT set was dried down and resuspended in 1.1 μl of HPLC-grade water.

### Sample Preparation for Label-Free Mass-Spec Single-Cell Proteomics

A total of eight 2-cell stage human embryos were used for label-free mass-spectrometry analysis. Frozen blastomeres that were collected in a 384-well plate were lysed by rapidly heating to 90° C. for 10 minutes and then cooling to 12° C. To each well (with a single blastomere or a water serving as the control), TG and TEAB were added to the cell lysate to final concentrations of 10 ng/μl and 100 mM, respectively. The plate was then kept at 37° C. for 3 hours to facilitate protein digestion. Each blastomere was dried down in a speed-vac and resuspended in 1.1 μl of HPLC-grade water for subsequent mass-spectrometry acquisition.

### Mass Spectrometry Acquisition Methods

TMT sets of single blastomeres were analyzed according to the SCoPE2protocol guidelines. Specifically, 1 μl out of 1.2 μl of each SCoPE2 pooled sample was loaded onto a 25 cm×75 1 μm IonOpticks Aurora Series UHPLC column (AUR2-25075C18A). Buffer A was 0.1% formic acid in water and buffer B was 0.1% formic acid in 80% acetonitrile/20% water. A constant flow rate of 200 nl/min was used throughout sample loading and separation. Samples were loaded onto the column for 20 min at 1% B buffer, then ramped to 5% B buffer over 2 min. The active gradient then ramped from 5% B buffer to 25% B buffer over 53 min. The gradient then ramped to 95% B buffer over 2 min and stayed at that level for 3 min. The gradient then dropped to 1% B buffer over 0.1 min and stayed at that level for 4.9 min. The total run time of each sample took 95 minutes total. All samples were analyzed by a Thermo Scientific Q-Exactive mass spectrometer from minutes 20 to 95 of the LC loading and separation process. Electrospray voltage was set to 2200 V and applied at the end of the analytical column. To reduce atmospheric background ions and enhance the peptide signal-to-noise ratio, an Active Background Ion Reduction Device (ABIRD, by ESI Source Solutions, LLC, Woburn MA, USA) was used at the nanospray interface. The temperature of the ion transfer tube was 250° C., and the S-lens RF level was set to 80.

Analysis of raw SCoPE2 and pSCoPE MS Data

Raw data were searched by MaxQuant (version 1.6.17) against a protein sequence database including all entries from the mouse or human SwissProt database (depending on which samples were being analyzed) and known contaminants such as human keratins and common lab contaminants (default MaxQuant contaminant list).

Within the MaxQuant search, trypsin digestion was specified and allowed for up to two missed cleavages for peptides having from 7 to 25 amino acids. Tandem mass tags (TMTPro 16plex) were specified as fixed modifications. Methionine oxidation (+15.99492 Da), and protein N-terminal acetylation (+42.01056 Da) were set as variable modifications. Second peptide identification was disabled. Calculate peak properties was enabled. All peptide spectrum matches (PSMs) and peptides found by MaxQuant were exported in the evidence.txt files. These evidence files were then analyzed together by DART-ID. The data from the files processed from DART-ID was then processed with the SCoPE2 pipeline with minor modifications, with filtering parameters including PEP<0.03 and PIF>0.8. Reverse matches and contaminants were also removed.

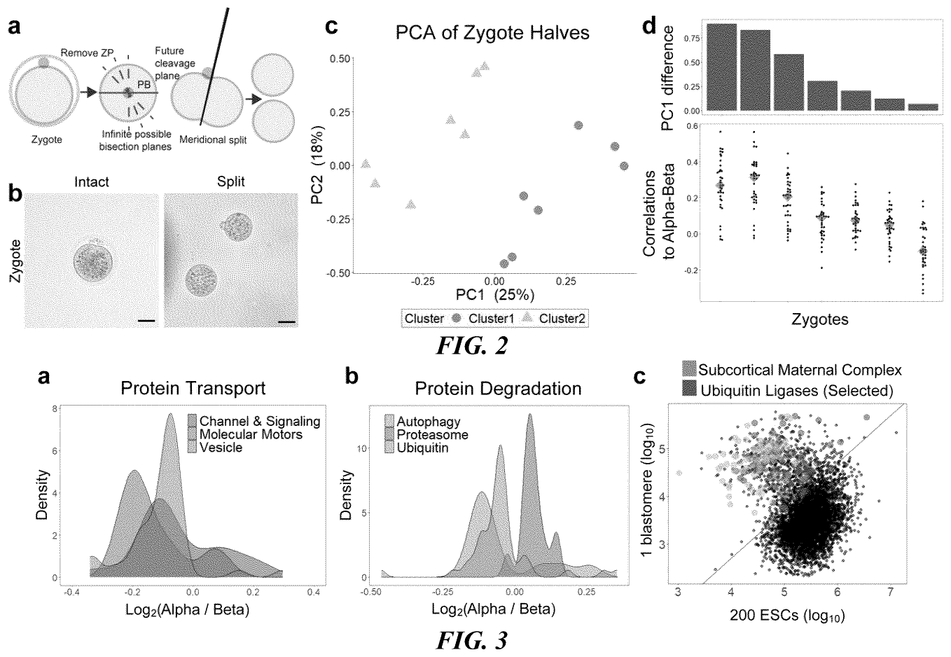

SCoPE2 pipeline is available at zenodo.org/record/4339954#.YnHcYSfMLOQ. The carrier and reference material in these experiments are clearly a different cell type from the single cells that were processed. Obtaining a large enough number of blastomeres to use as carrier and reference material for all the SCoPE2 sets was not possible. Although the cell types are different, many peptides that were heavily enriched in the blastomeres as shown in FIG. 2, panel c were still sequenced and quantified. These peptides and the corresponding proteins include classical markers of blastomeres and are biologically relevant, as evidenced by the analyses of mouse ESCs carriers vs blastomeres. The protein differences associated with alpha-beta asymmetry are also observed in the label free DIA experiments that did not use a carrier, suggesting that the choice of carrier is unlikely to strongly influence our results.

### Label-Free DIA Analysis and DIA-NN Search Parameters

Some human blastomeres were analyzed by label-free DIA using a 100 minute total gradient, of which 63 minutes were active (12 to 75 minutes). More specifically, the gradient used is as follows: 4% buffer B (minutes 0-11.5), 4%-8% buffer B (minutes 11.5-12), 8%-35% buffer B (minutes 12-75), 35%-95% buffer B (minutes 75-77), 95% buffer B (minutes 77-80), 95%-4% buffer B (minutes 80-80.1), 4% buffer B (minutes 80.1-100). Each duty cycle consisted of 2 MS1 windows with ranges from 480-1500 m/z. Each MS1 was followed by 3 MS2 windows spanning its m/z range (2× 1 MS1 full scan×3 MS2 windows). The size of the 6 MS2 windows in each duty cycle were variable and were as follows: 480-530 m/z; 530-590 m/z; 590-650 m/z; 650-750 m/z; 750-1000 m/z; 1000-1500 m/z. Each MS1 and MS2 scan was conducted at 140k resolving power, 3×10⁶ AGC maximum, and 600 ms maximum injection time for both MS1 and MS2 scans.

Raw data was searched with DIA-NN (version 1.8) against a protein sequence database that included entries from the human SwissProt database (SwissProt_human_09042017,containing 20,218 proteins). The fragment sizes were set from 200-1800 m/z, with N-terminal methionine excision enabled. The search was specified for trypsin digestion and the maximum number of missed cleavages was set to 1. Scan window radius was set 1, while the peptide lengths were set at 7-30 amino acids.

### K-Means Clustering

To estimate the stability of the cell classification that was accomplished via k-means clustering, the stability of cluster assignment was computed. Through 200 iterations in which the starting cell centroid was changed for each cluster, the probability of cluster assignment was estimated for each. The overwhelming majority of cells have a high probability of landing in the same cluster consistently when initial conditions are changed. There are some blastomeres (n=9) that seem to exhibit unstable cluster assignment, which were unable to be linked to division order, division timing, or division pattern. For simplicity's sake, these clusters were arbitrarily termed as alpha and beta. The same approach was used for both the human blastomeres from 2-cell embryos and the cut zygotes data.

Determining Differential Proteins Between Alpha- and Beta-Cell Types

Once cells were assigned to their respective classes via k-means clustering (k=2), next proteins that were significantly differentially abundant between the two groups of blastomeres were determined using a series of Kruskal-Wallis tests (effectively a Mann-Whitney-Wilcoxon test). At least three observations per group were required for each protein. P values of the tested proteins were adjusted for multiple hypotheses through the BH method to estimate the false discovery rate (FDR). A threshold of 5% FDR was implemented as the cutoff for significance for all results.

From these analyses, a list of differentially abundant proteins in 2-cell embryos was obtained and these proteins were used to plot two heatmaps in order to designate between the early and late 2-cell stages. The heat maps represent the proteins x blastomeres matrices. The columns of each heatmap were ordered by descending degree of asymmetry of sister blastomeres. The leftmost and rightmost columns correspond to blastomeres from the same embryo, a pattern that continues to the center of the heatmap.

Overall, 349 proteins are distinguishing the alpha-beta clusters. Out of this list, 163 proteins were quantified in the mouse zygote data, which is 47% of the defined alpha-beta proteins.

Overall, an average of 3586 peptides were quantified mapping to 1043 proteins in the mouse blastomere samples, and a mean of 2895 peptides mapping to 759 proteins in the human blastomere samples.

### Comparison Between Bulk Stem Cells and Mouse Blastomeres

Blastomere Peptide Enrichment. The reporter ion intensities (without any data processing) of a representative blastomere and its respective carrier on the log10 scale were plotted. In doing so, it was found that some peptides are much more abundant in one blastomere as compared to a 200-cell sample. In order to find what biological processes are enriched generally across mouse blastomeres as compared to ESCs, the precursor ratios of each blastomere to each respective ESC carrier were obtained. Then, the median across all blastomere-ESC pairs was taken to obtain the median ratio for each precursor. Then, these ratios were further collapsed to the protein level by taking the median across all peptides mapping to that protein. With this list, proteins were ranked from greatest to least ratios, then this ranked list was input into GOrilla using “single ranked list of genes” mode. From this output, it was found that protein transport and protein degradation are largely enriched in blastomeres.

### Protein Set Enrichment Analysis (PSEA) for Alpha vs Beta Comparison

To determine which processes are differential between alpha- and beta-cell clusters, protein sets were downloaded from MGI (MGI Data and Statistical Reports (jax.org)). These terms were filtered for proteins by Gene Symbols that were quantified in the mouse data. For each GO term, proteins by Gene Symbol that were associated with that GO term were collected into a single dataframe. That dataframe was further stratified into two groups: alpha- and beta-type. Each group was required to have greater than three observations. The two distributions per GO term were tested using the Kruskal Wallis test (effectively a Mann-Whitney-Wilcoxon test). P values of the tested GO terms were adjusted for multiple hypotheses, using the BH procedure to estimate the False Discovery Rate (FDR). GO terms were deemed significant if they passed the 5% FDR threshold.

For the mouse data, there were many GO terms that were significant (n=2898at 5% FDR, n=1499 at 5% FDR and with greater than 2 proteins associated with the term). In order to make sense of all the terms, the data was stratified into themes of protein degradation, protein transport, translation, and metabolism through filtering of the names of the GO terms. These themes were further grouped into sub-themes in the same manner. This approach was also used for the human 2-cell stage data, using protein sets defined for human data.

### Ribosomal Protein Analysis

For each ribosomal protein (RP) that was quantified, the Mann-Whitney-Wilcoxon test was used to understand whether the abundance of the particular RP was different between alpha and beta cells across all available mouse blastomeres (from early 2-cell to 4-cell stage). From these analyses, it was found that eight RPs are significantly differential (q-value<0.05). The distribution of these proteins' fold-changes between sister alpha and beta cells were plotted as boxplots at each stage.

### Vegetal Cell Analysis

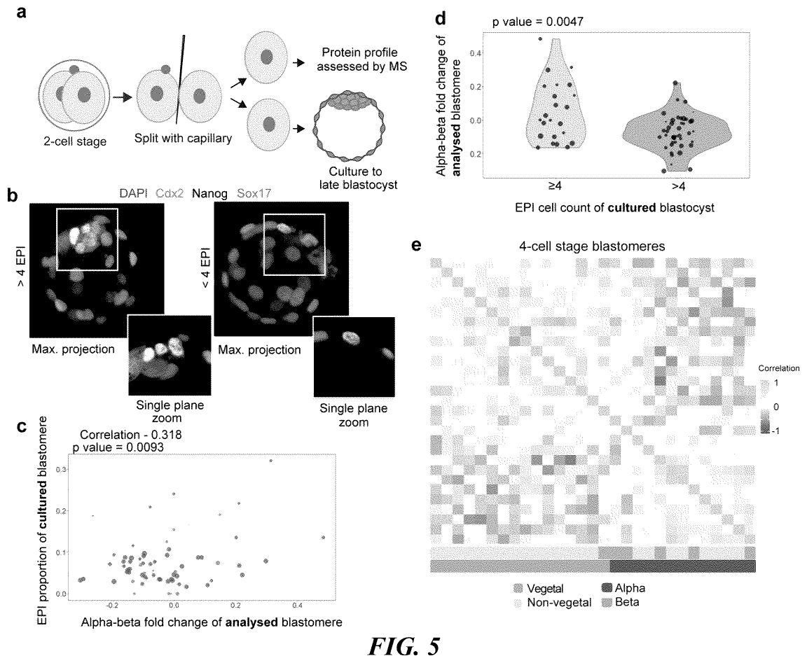

To identify whether alpha/beta polarization is associated with vegetal cell identity, 4-cell stage blastomeres were clustered with well differentiated alpha-beta character based on their relative protein levels, as shown in FIG. 5, panel e. As expected, the blastomeres clustered by alpha/beta polarization, and this clustering also portioned the vegetal cells. The statistical significance of this portioning was evaluated using the hypergeometric distribution to compute the cumulative probability (p-value) that the vegetal cells exhibit the observed association with alpha character or larger if sampled randomly.

### Across the Stages Analysis

To assess which biological processes are driving this trend, fold changes between sister blastomeres assigned to opposing classes were calculated for each protein. With these values, functionally related proteins that co-vary among the three stages were identified using spearman correlation analysis. For this analysis, each protein or each protein set was looped through and the stages (set to be numerical) were correlated to fold changes between alpha and beta blastomeres of normalized protein abundances. From each correlation, a p-value was also obtained. Protein sets were required to have more than two proteins and more than 50% of proteins quantified. These p-values were then corrected for multiple hypotheses.

### Comparison Between Human and Mouse 2-Cell Embryos

Pairwise Cell-to-Cell Correlation Heatmap. All proteins that were quantified in both datasets were used to calculate pairwise spearman correlations between human and mouse blastomeres at the 2-cell stage. The heatmap of spearman correlations was then plotted to have mouse blastomeres on the x-axis and human blastomeres on the y-axis. The human blastomeres are ordered in the same way as the dendrogram presented. The mouse blastomeres are clustered by their respective cluster-type, alpha or beta. Two distinct clusters were observed, meaning that human 2-cell stage embryos also exhibit a similar proteome asymmetry.

Intersected Protein Set Heatmap. Protein sets that were found to be differentially abundant between alpha and beta cells in mouse were intersected with protein sets that were found to be differentially abundant between the respective two clusters. Both heatmaps were hierarchically clustered on the rows (protein sets) and blastomeres on the columns were clustered according to the cell classification via k-means clustering. Each tile in the heatmap represents the median value on the log2 scale of that particular protein set in a particular blastomere.

### Cut Zygotes Analysis

Upon normalization and performing k-means clustering in the same manner as in the mouse and human data, all quantified proteins in the zygotes were used to perform principal component analysis. Each zygote half fell into a cluster opposite its partner half, which were simply termed in this case “Cluster 1” and “Cluster 2”. The difference in the first principal component (PC1) values were taken for each zygote pair and plotted in a descending order as a barplot. Then, protein fold changes between the partner cut pieces were calculated for each zygote (these values were calculated consistently by finding the difference between the zygote piece in Cluster 1 and in Cluster 2. These vectors of protein fold changes for each zygote were then correlated pairwise to protein fold changes of each mouse 2-cell stage blastomere (which were consistently calculated as the difference between alpha and beta cells), resulting in a correlation matrix. These results were plotted as distributions per zygote, with the median of each distribution highlighted as a purple diamond (FIG. 7, panel c).

In order to see the overall correlation between the zygote and mouse 2-cell blastomeres, the median fold change of each protein was calculated across all samples in respective groups (zygotes and 2-cell embryos). With these two vectors, a scatterplot of mouse 2-cell embryos protein fold-changes was plotted against the zygote fold-changes. The correlation of these vectors was positive (with a value of 0.44) and was highly significant (p-value=1.39×10⁹).

### Split Blastomere Experiment Analysis

Each blastomere that was used for MS analysis was prepared in a similar manner as was described in section titled “Sample preparation for label-free mass-spec single-cell proteomics” The MS acquisition was altered, so that peptides mapping to alpha-beta proteins were prioritized using prioritized SCoPE (pSCoPE), which was set up as described below, broadly following figure S4 from Huffman et al. 2023.

Gas phase fractionation (intensity-based quintiles spanning: 450-550, 550-623, 623-694, 694-788,788-1436 m/z respectively) was carried out to generate an empirical library using 5× TMT labeled mouse ESC carrier-reference runs. Post-acquisition, the runs were searched alongside all previous single cell DDA runs using Spectronaut (version 16.1), the generated spectral library was filtered at 5% FDR.

Subsequently, a 1× TMT labeled carrier reference sample was analyzed in DIA mode to record accurate retention times for precursors. The run was searched using Spectronaut (filtered at 1% FDR).

The inclusion list was generated using peptides confidently identified in the 1× run. Peptides were split into 3 tiers, the highest tier contained peptides belonging to alpha and beta proteins, while the following two tiers contained peptides split by intensity, confidence of identification and precursor ion fraction.

Prioritization was implemented using MaxQuant.Live Version 2.2.011. Targeting parameters were the same as in Supplementary table S13 (Method 5) of Huffman et al. 2023 (Nat. Methods (2023) doi: 10.1038/s41592-023-01830-1), with the exception that survey scan life cycle was set to 1500 ms, MS2 resolution was 70,000 and MS2 max injection time was 256 ms.

The resulting data was searched using MaxQuant and then normalized as described previously in the section titled “Analysis of raw SCoPE2 and pSCoPE MS Data”, except that the final protein x samples matrix was normalized relative to the mean of all analyzed cells. Then, for each blastomere, the median abundance of alpha proteins was divided by the median abundance of beta proteins to estimate the alpha-beta fold change of the analyzed cell. By designating which proteins are alpha and which are beta from previous clustering the “likeness” or polarization of blastomeres could be calculated, with a higher median alpha protein level indicating alpha identity, and higher median beta protein level indicating beta identity. After calculating the alpha-beta protein fold change or alpha-beta likeness of a blastomere, it can be inferred that the cultured sister blastomere is of the opposite identity, as 2-cell stage sisters consistently separated into opposing clusters. Each blastomere's fold change was then plotted against the proportion of epiblast cells in resultant blastocyst from the sister cell that was cultured. An overall positive trend is observed in the data. The distributions of alpha-beta fold changes were further analyzed by separating all blastomeres into two groups: (1) those whose sister blastomeres gave rise to blastocysts containing equal to or more than 4 epiblast cells and (2) those whose sister blastomeres gave rise to blastocysts with less than 4 epiblast cells, indicating the health of the embryo at this developmental stage.

### Example 2

### Proteomic Asymmetry in Blastomeres of 2-Cell and 4-Cell Embryos

This example investigates the protein abundance of 1) early 2-cell embryos, 2) late 2-cell embryos, when the major wave of zygotic genome activation occurs, and 3) 4-cell embryos (FIG. 1, panels a-c).

A method to separate and serially wash each blastomere from a single embryo was first established in a way that would avoid any protein contaminant from the culture media while retaining the relationship between blastomeres from the same embryo (FIG. 1, panels b and c). To quantify proteins in single blastomeres multiplexed Single Cell ProtEomics MS (SCoPE2) was used. In choosing cells for the isobaric carrier material, bulk blastomeres were first considered; however, this proved to be unfeasible, as each SCoPE2 set would require hundreds of blastomeres to be collected. It was reasoned that mouse embryonic stem cells (ESCs) would serve as adequate carrier cells, as carriers can differ from the single cell samples, ESCs represent a derivative of ICM and many cells can be harvested at a time.

To determine the relationship between individual 2-cell stage blastomeres, k-means clustering of the protein abundance normalized to the mean of individual 2-cell embryos was performed. The data were best explained by two clusters, which were termed alpha and beta (FIG. 7, panel a). From the analyses of 36 (15 early and 21 late) 2-cell mouse embryos, sister blastomeres were consistently classified into opposing clusters, with each embryo having an alpha and a beta blastomere with high confidence (FIG. 1, panel d and FIG. 1, panel e).

A set of 349 proteins was uncovered, which systematically differed in abundance between alpha and beta blastomeres in 2-cell embryos, out of an average of 1043 proteins quantified per single mouse blastomere (FIG. 1, panel d). These proteins included maternal factors implicated in zygotic genome activation, such as the cortical granule protein Padi6 and the ubiquitin E3 ligase RNF114, and cytoskeletal regulators such as Rdx53 and Cdc42, which are involved in the trophectoderm lineage later in development. This is believed to be the first report of systematic differences between the proteomes of sister blastomeres in the 2-cell mouse embryo.

It was found that the proteomic differences between sister blastomeres were more pronounced in late 2-cell embryos compared to early 2-cell embryos, as illustrated by the color scale in the heatmap (FIG. 1, panel d). However, the magnitudes of differential protein abundance between sister blastomeres varied (FIG. 1, panel d). This varying degree of difference between alpha and beta blastomeres is termed as the degree of asymmetry, which is observed to increase from the early to the late 2-cell stage. Overall quantitation variability of peptides mapping to the same proteins in each blastomere was low and unrelated to the degree of asymmetry (FIG. 7, panel b), and thus it is inferred that this degree of asymmetry is of biological origin.

Next, 4-cell embryos were investigated, whose blastomeres are known to have different molecular and developmental properties. 21 4-cell embryos were analyzed, in which a spectrum of the degree of asymmetry was observed among sister blastomeres from the same embryo (FIG. 7, panel c). Previously, proteins that consistently discerned alpha and beta blastomeres in 2-cell stage embryos were defined: proteins enriched in alpha blastomeres can be called alpha proteins and proteins enriched in beta blastomeres can be called beta proteins. Thus, the quantitation of these particular proteins can be used to quantify the observed differences among sisters. The variance of the distribution of the alpha and beta proteins in each blastomere were calculated, to observe the overall level of variability of protein levels (FIG. 7, panel d). In some 4-cell embryos, the level of variance was similar amongst sisters (e.g., embryo wAP539_29), while in others, the levels of variance were more different (e.g., embryo wAP563_24). This reflects the strength of alpha-beta polarization in each embryo. The ratio of the mean abundance of alpha and beta proteins in each blastomere was also calculated and used to indicate the “strength” of alpha and beta polarization. This approach indicated a range of strengths per blastomere (FIG. 7, panel e). Observing a higher abundance of alpha proteins in a particular blastomere indicates that the blastomere is more alpha. As an example, embryo wAP439_6 has two relatively strong alpha blastomeres and two relatively strong beta blastomeres. In another example, embryo wAP440_7 has one strong alpha blastomeres, a weak beta blastomere, and two strong beta blastomeres.

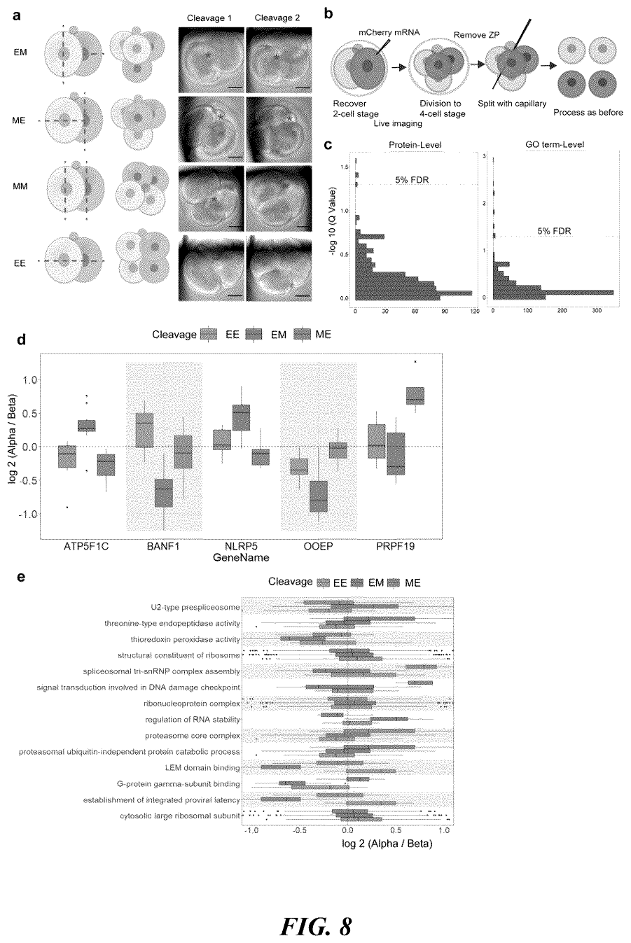

The 2-cell embryo can generate the 4-cell embryo via four distinct cleavage patterns, defined by the orientation of cell division (meridional—along the animal-vegetal axis or equatorial—perpendicular to animal-vegetal axis, with the animal-vegetal axis defined by the attached second polar body, which is the product of the second meiotic division of the oocyte upon fertilization) and order of division (FIG. 8, panel a). The cleavage pattern has been shown to impact the expression of heterogeneous factors at the 4-cell stage as well as the success of embryo development. To investigate whether the alpha-beta composition of the 4-cell embryos was related to a particular cleavage pattern, one sister blastomere was labeled in live 2-cell embryos by micro-injecting synthetic mCherry mRNA, and cleavage division patterns were recorded by time-lapse imaging, as described previously, individual 4-cell blastomeres collected and SCoPE2 performed (FIG. 8, panel b). It was found that few proteins and GO terms differed in abundance between alpha and beta blastomere clusters according to cleavage pattern; the statistical power of this analysis was however insufficient to reliably establish these differences (FIG. 8, panels c-e).

Overall, this example demonstrated consistent proteomic heterogeneity in sister blastomeres of 2-cell and 4-cell embryos, forming a molecular signature for alpha and beta cell clusters.

### Example 3

### Proteomic Asymmetry in the Zygote

This example investigates whether there is asymmetric protein distribution already within the zygote considering the alpha-beta asymmetry at the 2- and 4-cell stages demonstrated in Example 2.

Zygotes were manually bisected meridionally along the animal-vegetal axis, as this is the most frequent orientation of cleavage of the mouse zygote. Although the future cleavage plane will not always be recapitulated by experimental bisection of zygotes along the meridional axis, owing to the rotational symmetry of a spherical cell such as the zygote, there should nevertheless be some instances in which the physical cut approximates to the future cleavage plane (FIG. 2, panel a). Thus, it was expected that, if alpha-beta differences are already emerging at the zygote stage, some zygote halves will exhibit the alpha-beta protein differences while others will not. Pairs of zygote halves were collected and subsequently prepared and analyzed using SCoPE2 methods (FIG. 2, panels a, b).

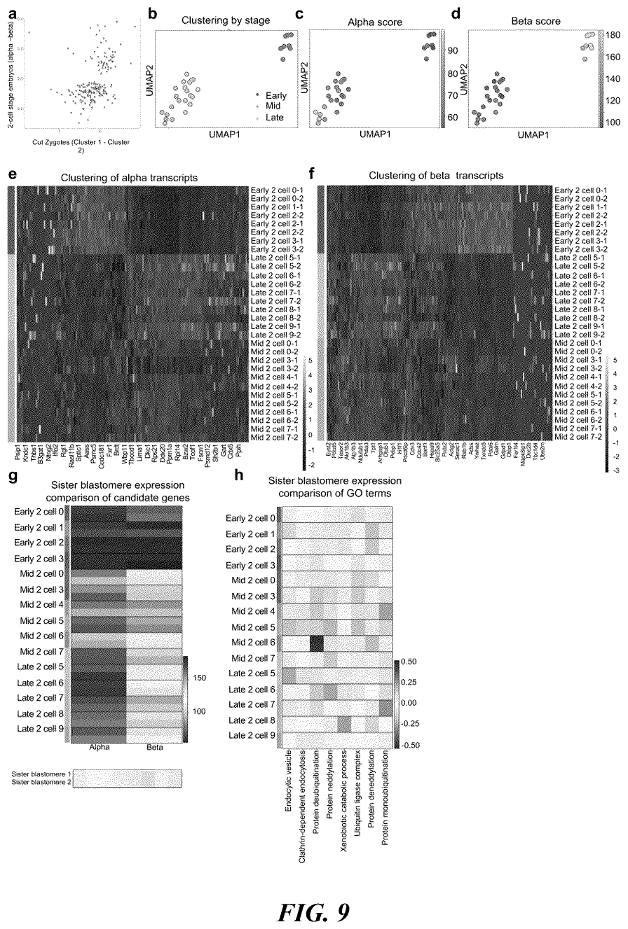

By performing the same proteomic and clustering analysis as previously done for 2-cell stage blastomeres, it was found that the zygote halves establish two clusters (FIG. 2, panel c). To test if the different zygote halves were related to the alpha and beta blastomeres, the 172 proteins that were 1) quantified in the zygote halves and 2) had significantly different abundance in alpha and beta blastomeres at the 2-cell stage were examined. For each protein, the median fold change in alpha versus beta cells and in zygote half 1 versus half 2 were determined. It was found that the Spearman correlation (r=0.45) is significant (p-value<1e-8, FIG. 9, panel a). Furthermore, when all pairwise correlations of these protein fold-changes between each zygote and embryo were taken, it was found that most zygotes had a median positive correlation with magnitude directly proportional to the degree of separation along PC1 (FIG. 2, panel d). This result is consistent with the expectation that variation in the plane of physical cutting along the meridional axis of the zygote will influence the sampled cross-section of protein distributions and thus the magnitude of the correlations. Differences in the proteomes of zygote halves correlate significantly with the protein differences between alpha and beta blastomeres at the 2- and 4-cell stage, which suggests that asymmetry likely stems from differential protein localization in the zygote. The data point towards the inheritance of asymmetry from the zygote to the 2-cell stage.

As asymmetry within the zygote and during stages prior to or during the major wave of zygotic genome activation was observed, it was hypothesized that proteome asymmetry might be driven by post-transcriptional mechanisms concerning maternal transcripts and proteins from the oocyte. When assessing previously published mouse single cell transcriptional data that span the early, mid and late 2 cell stage, stage dependent enrichment of the transcripts associated with alpha and beta proteins was observed (FIG. 9, panels b-d). A clear transcriptional signature where alpha and beta-associated transcripts show opposing expression patterns between sister blastomeres was not observed, but rather stage-dependent expression patterns, potentially reflecting zygotic genome activation (FIG. 9, panels e-g). Similarly, pathways which showed differences at the protein level do not exhibit similar patterns when only examining transcripts at this stage (FIG. 9, panel h). This suggests post-transcriptional mechanisms and maternal contributions may underlie the proteomic asymmetry reported here.

### Example 4

### Alpha and Beta Cells are Enriched for Different Biological Processes

This example investigates whether biological processes are different between alpha and beta cells.

To decipher processes that could be impacted by the differential abundance of proteins between alpha and beta blastomeres, protein set enrichment analysis (PSEA) was performed. It was found that protein degradation and protein transport processes were differentially abundant (q-values<0.05) in alpha and beta blastomeres (FIG. 3, panels a, b). In particular, ubiquitin- and autophagy-related terms were enriched in beta blastomeres, whereas proteasome-related terms were more enriched in alpha blastomeres. Protein transport terms (channel and signaling-related, molecular motors, and vesicle transport) were enriched in beta blastomeres, with processes related to molecular motors exhibiting the highest median fold difference.

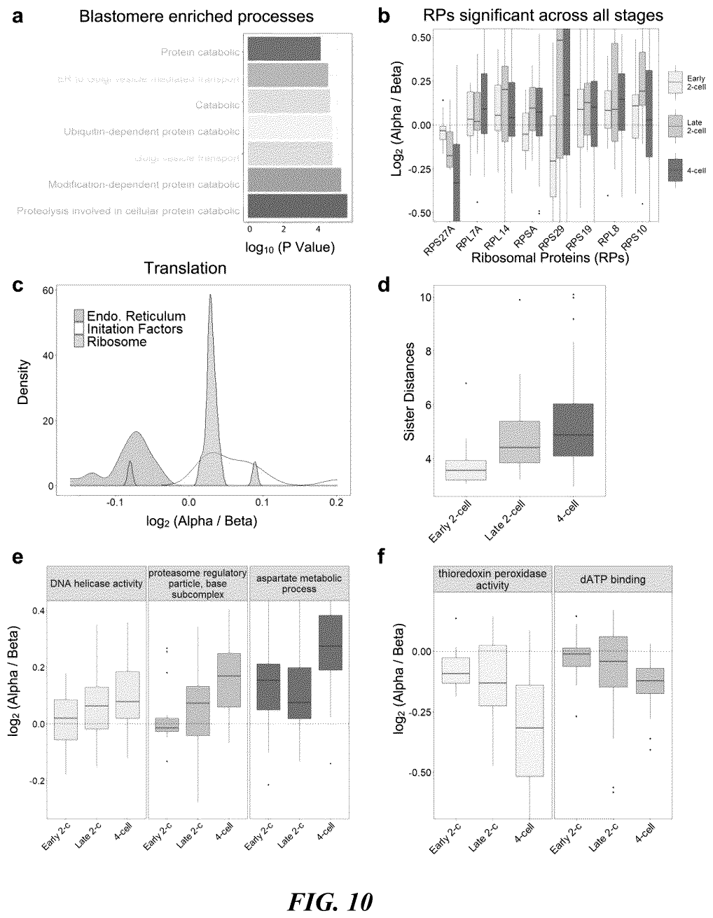

To understand how ESC proteomes compare with the blastomere proteomes, the peptide-level data of the carrier and single blastomeres were compared. Upon plotting the levels of shared peptides between single blastomeres and samples of 200 ESCs, a cloud of peptides that were much more abundant—up to 10-fold higher—in single blastomeres (a representative plot is shown in FIG. 3, panel c) was found. The overall range of peptide abundances should scale with sample size, and so it was surprising to see many peptides exhibiting much higher abundance in single blastomeres as opposed to 200 ESCs. These peptides derive in part from the subcortical maternal complex (SCMC), a maternally encoded multiprotein complex that is critical for early development. The cloud of outliers also includes peptides related to ubiquitin ligases. This high abundance of ubiquitin ligases is further confirmed by systematic GO term analysis across all single blastomeres, which revealed strong enrichment (relative to ESCs) for peptides implicated in protein degradation and protein transport (FIG. 10, panel a), consistent with the known importance of proteasomal degradation during this period of embryonic development, encompassing maternal protein degradation, alongside zygotic genome activation and subsequent novel zygotic protein synthesis. As proteins mapping to these similar processes were also found to be differentially abundant between alpha and beta blastomeres, these analyses furthermore underscore their potential association with inter-blastomere proteomic heterogeneity.

### Example 5

### Dynamics of Alpha and Beta Differences

This example investigates whether differences between alpha and beta blastomeres change during development.

Mouse embryos at the zygote and early 2-cell stage depend largely on maternally inherited cellular components, including proteins, mRNAs and ribosomes, prior to the major wave of zygotic genome activation. Different ribosomal stoichiometries have been suggested to contribute to ribosome-mediated translational control during early embryogenesis and in ESCs. Therefore, this example first assessed whether the ribosomal protein (RP) levels in the samples might be consistent with this idea.

It was noticed that the levels of most RPs were slightly, yet statistically significantly, elevated in alpha blastomeres compared to beta cells blastomeres in early 2-cell, late 2-cell and 4-cell embryos (FIG. 10, panel b). An exception was RPS27A, whose enrichment in alpha blastomeres increased during development. Proteins involved in translation initiation factors were also more abundant in alpha blastomeres as compared to beta blastomeres, whereas GO terms related to endoplasmic reticulum showed the opposite trend (FIG. 10, panel c).

To explore whether differences between alpha and beta blastomeres change during development, the Euclidean distances between alpha and beta blastomeres from the same embryo were first calculated, using proteins that were quantified in every cell analyzed. As noted in FIG. 1, panel d, it was observed that the degree of proteomic differences between alpha and beta cell clusters increased significantly during early development (FIG. 10, panel d), suggesting sisters may increasingly diverge across stages.

The increased sister divergence across time can be attributed to proteins that are either consistently decreasing or increasing across the developmental stages. The 324 proteins which were quantified in both early and late 2-cell embryos and also deemed to be differentially abundant between alpha and beta blastomeres were considered, and it was found that 278 proteins preserve cluster identity and are either consistently increasing or decreasing over time. When taking into account the 4-cell stage, a smaller number of proteins (254) continue to preserve cluster identity, of which 108 are monotonically changing across the three timepoints.

To explore which processes are showing monotonic changes across time, PSEA was performed using Spearman correlations across the three developmental timepoints and the corresponding protein fold-changes between alpha and beta cells from each embryo. Representative protein sets that were decreasing in magnitude across the stages were thioredoxin peroxidase activity and dATP binding (FIG. 10, panels e, f). It was also found that the proteasome regulatory particle is increasing in magnitude within alpha blastomeres, just as found in the previous PSEA (FIG. 2, panel b). In addition, representative processes of aspartate metabolic and DNA helicase activity exhibit the same behavior. Overall, these analyses attempted to discern which molecular pathways could be driving the increased divergence between alpha and beta as development progresses.

### Example 6

### The Role of Beta Proteins in Lineage Fate

This example investigates a role for three of the differentially abundant proteins in lineage specification. Specifically, Nedd8, Gps1 and PSMC4 were chosen based on their differential abundance between alpha and beta blastomeres and their putative roles in preimplantation development. For instance, activation of the ubiquitin-like protein Nedd8, has been implicated in formation of the ICM. Gps1 (Cops1) is a subunit of the COP9 signalosome, which is involved in deneddylation and has been implicated in naive pluripotency and epiblast survival. Gps1 is also a putative regulator of expression of the transcription factor Oct4 in human ESCs, but has not been studied in mammalian embryos before. PSMC4 is a component of the 26S proteasome, and a previous report showed that PSMC4 deficient embryos do not develop into blastocysts. Nedd8 and Gps1 have higher abundance in beta blastomeres, whilst PSMC4 has a higher abundance in alpha blastomeres.

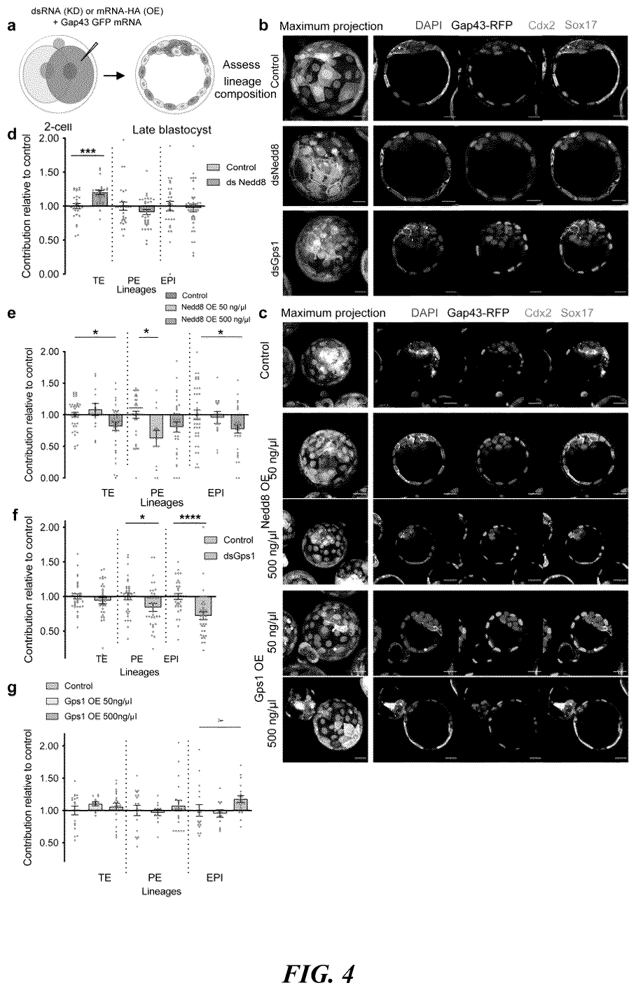

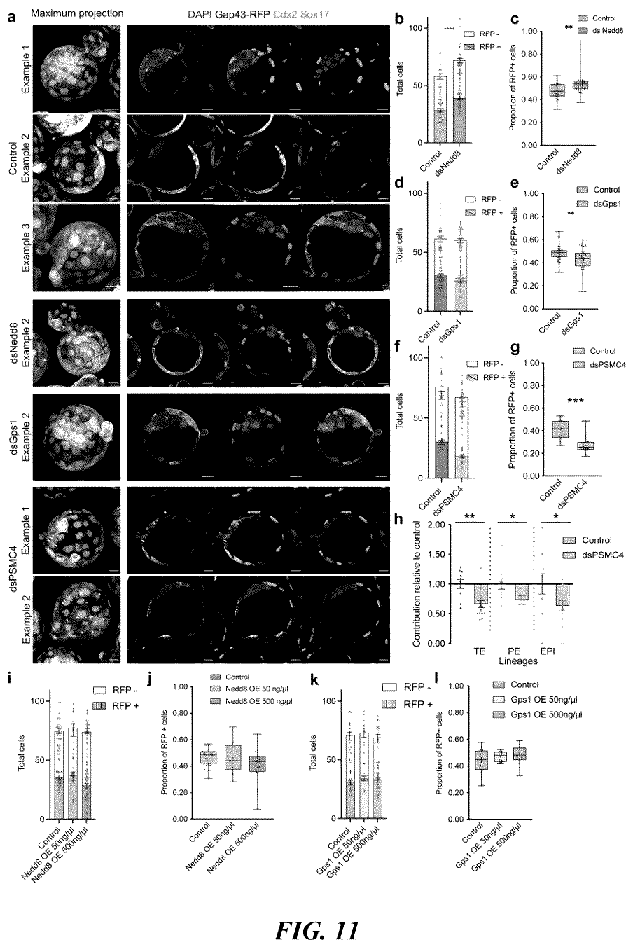

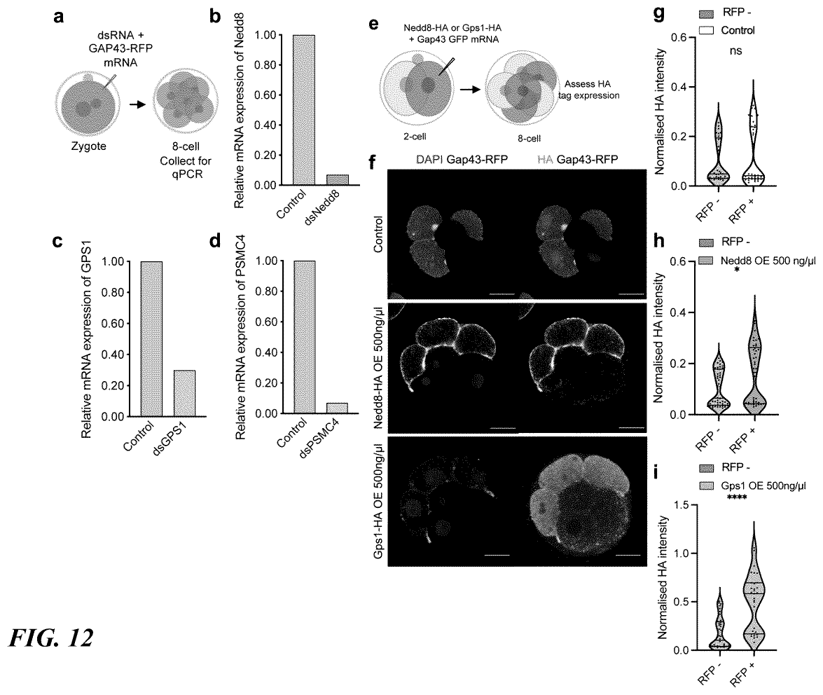

RNAi was used to knockdown (KD) candidates in one blastomere of the 2-cell embryo and evaluated the consequences for lineage contribution and development of the embryo for 3 days, until the blastocyst stage. Briefly, one of the two blastomeres was randomly micro-injected with dsRNA targeting each candidate (or eGFP as a control) and also with mRNA encoding Gap43-RFP to label and follow the progeny of the injected blastomere, using a protocol previously established (FIG. 4, panel a). Following micro-injection, embryos were cultured to the late blastocyst stage, fixed and stained for Cdx2 to identify the trophectoderm lineage and Sox17 to identify the primitive endoderm (FIG. 4, panel b, FIG. 11, panel a). The KD efficiency was validated by micro-injecting the dsRNA into zygotes, and observing a reduction in mRNA levels 48 hrs later in 8-cell embryos by qRT-PCR (FIG. 12, panels a-d). As a complementary approach, overexpression (OE) studies were performed, in which one blastomere of a 2-cell embryo was co-injected with mRNAs encoding the candidate protein fused to an HA tag and a Gap43-RFP to label the progeny of the injected cell, and cultured to the late blastocyst stage (FIG. 4, panels a and c). The OE of candidates was validated by assessment of HA tag expression by immunofluorescence (FIG. 12, panels e-i).

The data suggest that the blastocyst cell number and the proportion of RFP-positive cells in the blastocyst was slightly but statistically significantly higher upon Nedd8 knockdown (KD) (FIG. 11, panels b and c). Moreover, Nedd8 KD significantly increased the frequency of RFP-positive cells in the trophectoderm relative to controls, but did not have a significant effect on the epiblast or primitive endoderm (FIG. 4, panel d). In comparison it was found that the total number of cells in the blastocyst and the proportion of RFP-positive cells in the blastocyst did not differ upon Nedd8 OE (FIG. 11, panels i and j), but Nedd8 OE significantly decreased the frequency of RFP-positive cells in the trophectoderm and epiblast (FIG. 4, panel e). It was inferred that Nedd8 may inhibit the specification and/or proliferation of trophectoderm cells.

Gps1 KD did not reduce blastocyst total cell number (FIG. 11, panel d) but reduced the proportion of RFP-positive cells in the blastocyst (FIG. 11, panel e), particularly in the epiblast, with a less significant reduction in primitive endoderm and no significant reduction in trophectoderm contribution (FIG. 4, panel f). Gps1 OE did not impact the total number of cells in the blastocysts or the proportion of RFP-positive cells contributing (FIG. 11, panels k and l) but led to an increase in contribution to the epiblast, rather than a reduction as observed when knocking down Gps1 expression (FIG. 4, panel g). These data suggest that Gps1 may promote specification and/or proliferation of epiblast cells, and are consistent with the suggested role of Gps1 in promoting pluripotency.

The data suggest that PSMC4 KD reduced the cell number and proportion of RFP-positive cells in all lineages of the blastocyst (FIG. 10, panels f, g and h), suggesting that PSMC4 can promote proliferation of uncommitted cells.

Thus, expression of Nedd8 and Gps1, which are more abundant in beta blastomeres, impacts the trophectoderm and epiblast lineage, respectively, in the blastocyst. The phenotypes observed suggest that Gps1 and Nedd8 can play a role in promoting the epiblast fate and suppressing the trophectoderm fate respectively. On the other hand, reduction of PSMC4, which is more abundant in alpha blastomeres, impacted all lineages, perhaps, and without being bound by any particular theory, reflecting the importance of the proteasome as the downstream effector of protein degradation. Inhibition of the proteasome has also previously been found to delay DNA replication and cleavage divisions, fitting with the observations. These results indicate that the differential proteins identified by SCoPE2 can impact lineage composition, and point towards the importance of protein degradation pathways during preimplantation development.

### Example 7

### The Alpha Versus Beta Identity of Blastomeres Correlates with Differences in Developmental Potential

This example investigates the correlation between the identity of the 2-cell blastomere (an alpha cell and a beta cell) and its subsequent blastocyst development.

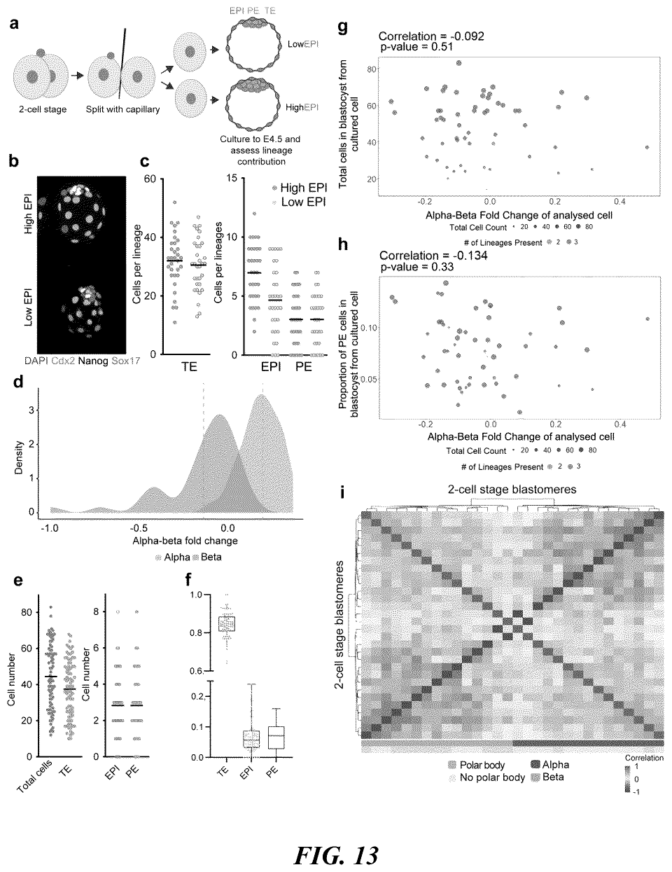

It was previously shown that the developmental potential and subsequent fate of 2-cell stage sister blastomeres are unequal. Specifically, separated sister blastomeres of the 2-cell mouse embryo show discordance in their ability to give rise to a viable embryo and show variation in epiblast size, with one blastomere giving rise to more epiblast cells than its sister (FIG. 13, panels a-c). To determine if alpha and beta blastomeres differ in their developmental potential, sisters were separated from 2-cell embryos and one sister was analyzed by MS (SCoPE2 or pSCoPE) to determine its identity (alpha or beta, as quantified by calculating the alpha-beta protein fold change) and the other sister cell cultured to the blastocyst stage (FIG. 5, panel a and b, FIG. 13, panel d). The observations that sister blastomeres always fall into opposing clusters, with each 2-cell embryo containing an alpha and a beta cell (FIG. 1, panel d) were taken into account. Thus, it was inferred that identity to the cultured sister cell was the opposite to its sister that was analyzed by MS (FIG. 5, panel a). The identity of the 2-cell blastomere was correlated with its subsequent blastocyst development including total cell number, and number of cells in each of the three lineages (FIG. 5, panel c, FIG. 13, panels e-h). It was found that blastomeres with a higher beta identity gave rise to blastocysts with a higher proportion of epiblast cells (FIG. 5, panel c). Crucially, beta blastomeres are more likely to give rise to blastocysts with 4 epiblast cells, the minimum number required for successful further development (FIG. 5, panel d). These data agree with the knockdown and overexpression data (FIG. 4), which suggest that beta proteins support epiblast formation and/or inhibit trophectoderm formation. It was recently reported that inheritance of the polar body at the 2-cell stage predicts developmental potential, with the sister inheriting the second polar body, giving rise to more ICM cells. In agreement with this observation, when examining whether there was any relation between inheritance of the polar body and alpha or beta identity, it was found that the sister associated with the polar body was significantly more likely to be a beta cell (FIG. 13, panel i).

At the 4-cell stage, vegetal blastomeres (the blastomere that is furthest away from the polar body, which defines the animal pole of the embryo), are known to be significantly biased to the trophectoderm and have a lower developmental potential. Therefore, the alpha-beta classification of vegetal blastomeres in the 4-cell stage embryos (FIG. 5, panel e) was examined. Plotting the pairwise cell correlations for blastomeres from 4-cell embryos, revealed two clusters corresponding to the alpha-beta polarization of each blastomere. It was found that vegetal blastomeres were significantly more likely to be alpha than their non-vegetal counterparts. Thus, the alpha-beta identity of a blastomere correlates with differences in developmental potential, with beta blastomeres having a higher developmental potential and alpha blastomeres a lower developmental potential.

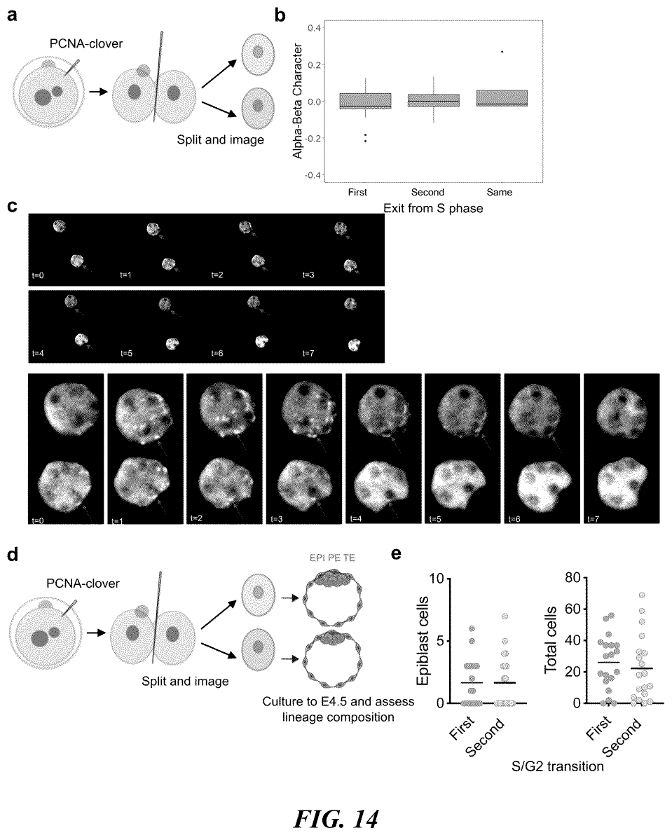

Since 2-cell stage blastomeres are often asynchronous in their cell cycle progression, the example then investigated whether the divergence of alpha and beta blastomeres can be correlated with the asynchrony in developmental timing. In order to test this, zygotes were micro-injected with PCNA-clover mRNA, foci of which indicate S phase progression, and following cleavage to the 2-cell stage, assessed by live imaging to see which sister had completed S phase and entered G2 first (FIG. 14, panels a, b). After imaging, single 2-cell stage blastomeres were collected for subsequent MS analysis using pSCoPE. When normalizing the MS data within each embryo, sister blastomeres consistently fell into opposite clusters, as observed before. However, it was found that the distributions of alpha-beta polarization are not significantly different between ‘early’ and ‘later’ exit from S-phase, and therefore a relationship between exit from S-phase and alpha-beta identity was not observed (FIG. 14, panel c).

Concurrently, the experiment was repeated but blastomeres were allowed to develop further to the blastocyst stage, at which point they were fixed and stained for lineage markers (FIG. 14, panel d). As alpha and beta blastomeres give rise to blastocysts with differing cell numbers of epiblast cells (FIG. 5, panels c and d), the epiblast cell number from blastocysts arising from sister 2-cell blastomeres which completed S phase ‘first’ or ‘second’ was compared. No difference in the distribution of epiblast cell numbers was observed (FIG. 14, panel e). This supports the finding that cell cycle asynchrony, as assessed by S phase exit, is unlikely to differentiate alpha and beta cells.

### Example 8

### Proteomic Asymmetry is Conserved in Human 2-Cell Stage Embryos

This example investigates if the protein patterns defining alpha and beta blastomeres in mouse embryos are conserved in human embryos.

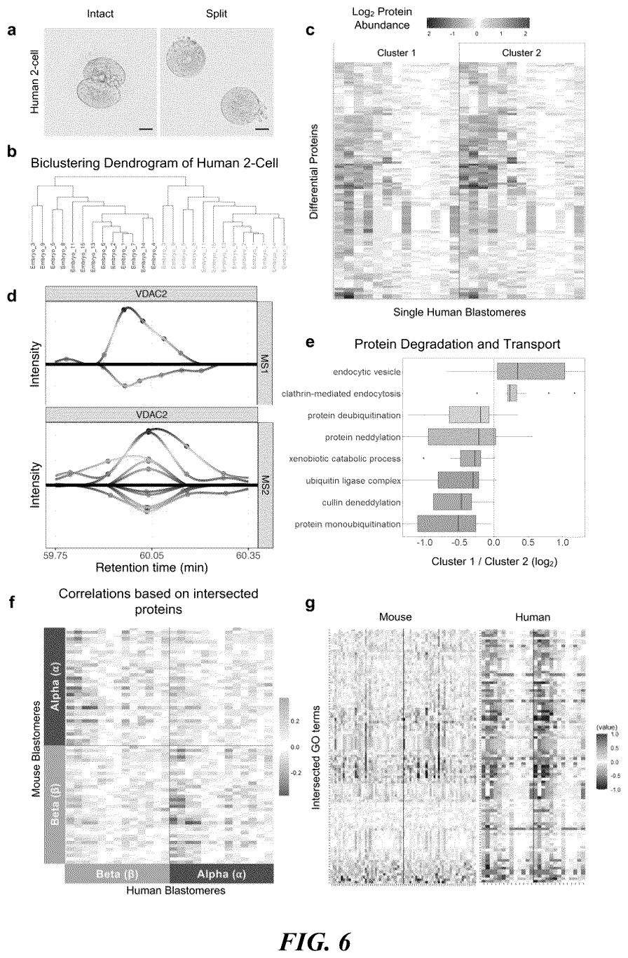

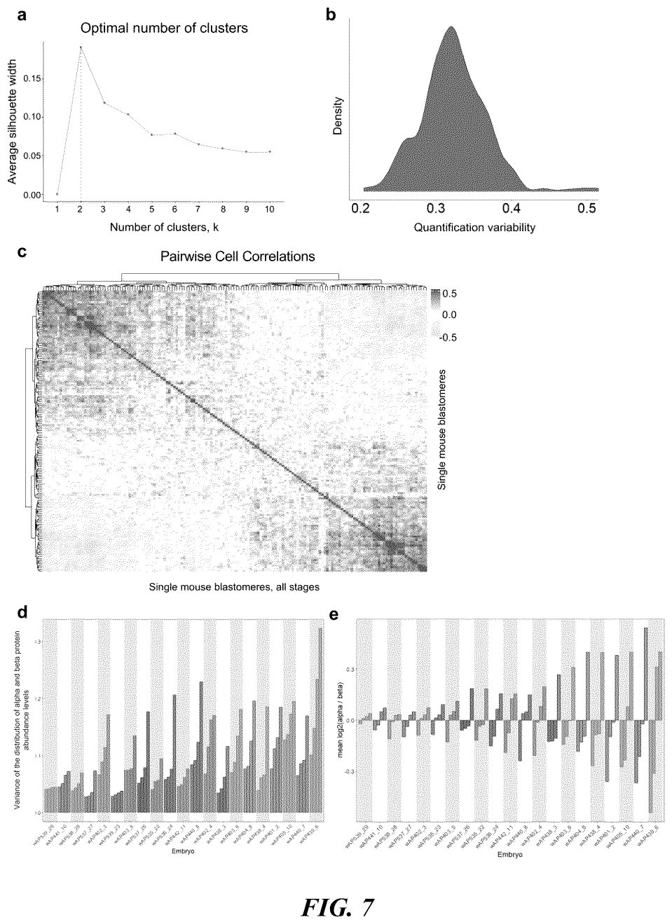

To this end, 2-cell human embryos were examined, which were donated to the research via IVF clinics (FIG. 6, panel a). Since access to human embryos at the early cleavage stages is extremely limited for technical reasons, the number of embryos examined was fewer than for mouse. As these samples are extremely precious, two orthogonal single-cell MS methods were used: label-free data-independent acquisition or SCoPE2 data-dependent acquisition methods. These methods have different systematic biases, and thus concordant results are unlikely to be due to a methodological bias.

The k-means clustering approach was performed as implemented with the mouse blastomeres and remarkably, it was found that sister 2-cell human blastomeres also fell into two opposing clusters (FIG. 6, panel b). Between the two cell clusters, 113 differentially abundant proteins were identified at 1% FDR (FIG. 6, panel c). To assess the raw MS data more closely, the ion chromatogram (XIC) was extracted for one of the proteins with the highest fold difference, VDAC2, a voltage-dependent anion-selective channel protein. Both the MS1 and the MS2 XICs indicate consistent differential abundance of VDAC2 across the sister blastomeres (FIG. 6, panel d). This observation of VDAC2 being increased in one blastomere fits with the observations from mouse embryos in which protein transport was highly differential between alpha and beta blastomeres. The functional validation of the role of the identified differentially distributed proteins in human embryos at such an early developmental stage is unfeasible.

To further characterize the clusters of human blastomeres, PSEA was performed. Similar to the corresponding analysis of mouse blastomeres, the results again indicated that proteins involved in degradation and transport were differentially abundant in sister blastomeres of 2-cell human embryos (FIG. 6, panel e). Ubiquitin-related proteins were enriched in one cell cluster type, whereas vesicle-related proteins were enriched in the opposing cluster, similar to mouse alpha versus beta blastomeres, respectively.

The two clusters observed in human embryos suggest conservation of the existence of proteomic heterogeneity at the 2-cell stage across human and mouse, therefore next the concordance was tested more directly. Using 877 proteins whose orthologues were quantified in both mouse and human 2-cell stage embryos, the pairwise correlations between mouse and human blastomeres (FIG. 6, panel f) were calculated. The results indicated two distinct clusters, which support that alpha-beta intra-embryo protein differences are conserved across mouse and human and allowed one to extend the alpha and beta annotation to human blastomeres. Additionally, the GO terms that were significantly differential between alpha and beta cell clusters and that were shared between the mouse and human data were examined and significant concordance was found among the GO term directionality in the two organisms (FIG. 6, panel g).

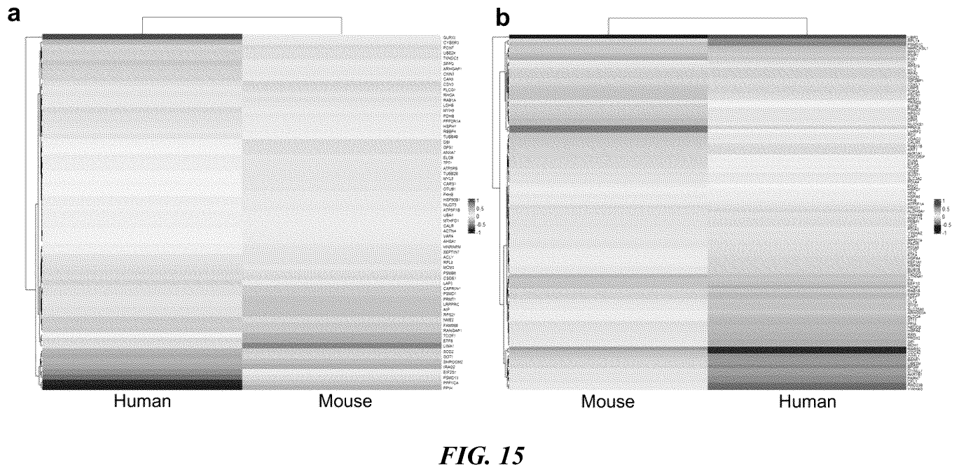

To further understand the similarities and differences between the mouse and human blastomeres, the space of analysis was narrowed into proteins that were found significantly differential between alpha and beta 2-cell mouse blastomeres. Then, the fold changes between the median levels of each protein for alpha and beta cells in both human and mouse were estimated. Through the comparison of these fold-changes, 68 proteins that change in the same direction in both human and mouse blastomeres, and 98 proteins that exhibit opposite directionality (FIG. 15, panels a and b) were identified. Such analysis suggests that whilst there is some conservation of how the alpha-beta proteins behave across species, there are also species differences. Therefore, intra-embryo proteomic differences found in the mouse are also present in human embryos both at the level of differentially abundant proteins and enrichment of protein sets representing different themes of biological processes.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for determining protein composition of a mammalian embryo in vitro, comprising:
culturing one or more mammalian embryos at the zygote stage in an embryo culture media until the one or more mammalian embryos reach at least early 2-cell stage;
separating sister blastomeres of a mammalian embryo into single blastomeres;
subjecting the single blastomeres to single-cell mass-spectrometry to obtain a proteomic profile of each single blastomere; and
identifying a first set of proteins differentially abundant in a first single blastomere and a second set of proteins differentially abundant in a second single blastomere, both blastomeres derived from a same mammalian embryo.

2. The method of claim 1, wherein the one or more mammalian embryos at the zygote stage are cultured in the embryo culture media for at least about 1-3 hours or about 6-12 hours.

3. The method of claim 1, wherein the one or more mammalian embryos reach a late 2-cell blastomere stage, a 4-cell blastomere stage, or an 8-cell blastomere stage.

4. (canceled)

5. (canceled)

6. (canceled)

7. The method of claim 1, wherein the embryo culture media comprises physiological salts, a carbon source, an antibiotic, and a buffer, optionally, the carbon source is glucose.

8. (canceled)

9. The method of claim 7, wherein the embryo culture media further comprises non-human serum or serum substitute.

10. (canceled)

11. The method of claim 7, wherein the embryo culture media comprises (i) physiological salts, energy substrates, bicarbonate or HEPES, essential amino acids, glutamine dipeptide, human serum albumin, EDTA, gentamicin, or any combination thereof; and/or (ii) sodium chloride, potassium chloride, calcium chloride, potassium phosphate, magnesium sulfate, sodium bicarbonate, glucose, sodium lactate, sodium pyruvate, amino acids, EDTA, gentamicin sulfate, or any combination thereof.

12. (canceled)

13. The method of claim 1, wherein separating blastomeres of the one or more mammalian embryos into single blastomeres comprises removing the zona pellucida of the one or more mammalian embryos and bisecting the embryos, optionally further comprising washing the single blastomeres.

14. The method of claim 1, wherein subjecting the single blastomeres to single-cell mass-spectrometry comprises lysing the single blastomeres using a physical or mechanical lysis process, and contacting the cell lysate with a trypsin enzyme.

15. The method of 14, wherein subjecting the single blastomeres to single-cell mass-spectrometry comprises labeling digested peptides by isobaric mass tags (TMT).

16. The method of claim 14, wherein the single-cell mass-spectrometry comprises preparing an isobaric carrier, and wherein preparing the isobaric carrier comprises lysing embryonic stem cells and contacting the cell lysate with a trypsin enzyme.

17. (canceled)

18. (canceled)

19. (canceled)

20. (canceled)

21. The method of claim 1, further comprising determining the degree of asymmetry in the single blastomeres derived from the same mammalian embryo.

22. (canceled)

23. (canceled)

24. (canceled)

25. (canceled)

26. The method of any one of claim 1, wherein the second set of proteins differentially abundant in the second single blastomere comprise proteins that support the epiblast formation and suppress the trophectoderm formation.

27. The method of claim 1, comprising identifying the single blastomere having a higher developmental potential.

28. The method of claim 1, wherein the first set of proteins differentially abundant in the first single blastomere comprises PSMC4, and the second set of proteins differentially abundant in the second single blastomere comprises Nedd8 and Gps1.

29.-34. (canceled)

35. The method of claim 1, wherein the mammalian embryo is a human embryo or a mouse embryo.

36. (canceled)

37. A method for detecting perturbation-induced change in mammalian embryo cells, comprising:
introducing a perturbation to a mammalian embryo at the zygote stage;
determining a protein composition of the mammalian embryo according to claim 1; and
detecting the perturbation-induced change in the protein composition of single blastomeres, optionally the detecting comprises comparing the protein composition obtained in the presence of the perturbation with a protein composition obtained in the absence of the perturbation.

38. The method of claim 37, wherein the perturbation is a physical condition or a chemical condition.

39. The method of claim 37, wherein the perturbation comprises a genetic perturbation, a drug candidate, an absence or presence of a test agent, an increase or decrease of temperature, light, pressure, pH value, or a combination thereof.

40. The method of claim 37, wherein introducing the perturbation to the mammalian embryo comprises (i) contacting the mammalian embryo with the perturbation in a culture media or culturing the mammalian embryo in a culture media in the presence of the perturbation, or (ii) performing RNA interference or CRISPR-Cas gene editing.

41. (canceled)

42. (canceled)

43. The method of claim 37, wherein detecting the perturbation-induced change on the protein composition of sister blastomeres comprise detecting the abundance of one or more proteins selected from Nedd8, Gps1, PSMC4, or a combination thereof in the sister blastomeres.

44.-48. (canceled)


SEQUENCE LISTING (22 sequences)

SEQ ID NO: 1 - DNA, 29 bp, synthetic construct
  source 1..29: mol_type = other DNA
acgtgaattcatggtgagcaagggcgagg

SEQ ID NO: 2 - DNA, 34 bp, synthetic construct
  source 1..34: mol_type = other DNA
acgtggatccctacttgtacagctcgtccatgcc

SEQ ID NO: 3 - DNA, 49 bp, synthetic construct
  source 1..49: mol_type = other DNA
acgttaatacgactcactatagggattctatgaatccaagtatgcctca

SEQ ID NO: 4 - DNA, 47 bp, synthetic construct
  source 1..47: mol_type = other DNA
acgttaatacgactcactatagggacagctgctctcagaatcatagc

SEQ ID NO: 5 - DNA, 44 bp, synthetic construct
  source 1..44: mol_type = other DNA
acgttaatacgactcactataggggggagaagcagcactctagc

SEQ ID NO: 6 - DNA, 44 bp, synthetic construct
  source 1..44: mol_type = other DNA
acgttaatacgactcactatacggtctggtgtcccagagagtga

SEQ ID NO: 7 - DNA, 41 bp, synthetic construct
  source 1..41: mol_type = other DNA
acgtaatacgactcactataggggcccaggaggaggtgaag

SEQ ID NO: 8 - DNA, 43 bp, synthetic construct
  source 1..43: mol_type = other DNA
acgtaatacgactcactatagggatcgatgccaatctgcttgt

SEQ ID NO: 9 - DNA, 20 bp, synthetic construct
  source 1..20: mol_type = other DNA
cgtattgggcgcctggtcac

SEQ ID NO: 10 - DNA, 20 bp, synthetic construct
  source 1..20: mol_type = other DNA
atgatgacccttttggctcc

SEQ ID NO: 11 - DNA, 22 bp, synthetic construct
  source 1..22: mol_type = other DNA
gatccatgtcaagtctcctcct

SEQ ID NO: 12 - DNA, 20 bp, synthetic construct
  source 1..20: mol_type = other DNA
ctgttggctggagtcagctc

SEQ ID NO: 13 - DNA, 21 bp, synthetic construct
  source 1..21: mol_type = other DNA
tactggtgggagaatgtgagg

SEQ ID NO: 14 - DNA, 22 bp, synthetic construct
  source 1..22: mol_type = other DNA
taagacagggaagcacacatga

SEQ ID NO: 15 - DNA, 19 bp, synthetic construct
  source 1..19: mol_type = other DNA
cagcactgtccgtgtctcg

SEQ ID NO: 16 - DNA, 24 bp, synthetic construct
  source 1..24: mol_type = other DNA
ctgctcgtccttgatatactcctc

SEQ ID NO: 17 - DNA, 16 bp, synthetic construct
  source 1..16: mol_type = other DNA
cgcgtcaggccaacat

SEQ ID NO: 18 - DNA, 27 bp, synthetic construct
  source 1..27: mol_type = other DNA
acgtaagcttccgcgtcaggccaacat

SEQ ID NO: 19 - DNA, 48 bp, synthetic construct
  source 1..48: mol_type = other DNA
tcatacccatacgatgttccagattacgctcatgttggtactcatgcg

SEQ ID NO: 20 - DNA, 38 bp, synthetic construct
  source 1..38: mol_type = other DNA
acgtgaattcatgctaattaaagtgaagacgctgactg

SEQ ID NO: 21 - DNA, 38 bp, synthetic construct
  source 1..38: mol_type = other DNA
tacccatacgatgttccagattacgctctgcccaagac

SEQ ID NO: 22 - DNA, 40 bp, synthetic construct
  source 1..40: mol_type = other DNA
acgtggatcctcatacccatacgatgttccagattacgct